(19) United States
(12) Patent Application Publication
(10) Pub. No.: US 2025/0160636 A1
(43) Pub. Date: May 22, 2025

(54) Mitigating Ocular Toxicity

(71) Applicant: GlaxoSmithKline Intellectual Property Development Limited, Stevenage (GB)

(72) Inventors: Mala Kiran TALEKAR, Collegeville, PA (US); Jeffery Lanier PAINTER, JR., Collegeville, PA (US)

(73) Assignee: GlaxoSmithKline Intellectual Property Development Limited, Stevenage (GB)

(21) Appl. No.: 18/835,384

(22) PCT Filed: Feb. 7, 2023
(86) PCT No.: PCT/IB2023/051085

§ 371 Date: Aug. 2, 2024

Related U.S. Application Data
(60) Provisional application No. 63/307,922, filed on Feb. 8, 2022.
(60) Provisional application No. 63/323,127, filed on Mar. 24, 2022.

(51) Int. Cl. A61B 3/00; A61B 3/028; C07K 16/28; G16H 20/10; G16H 50/30

(52) U.S. Cl. CPC A61B 3/0033; A61B 3/028; C07K 16/2878; G16H 20/10; G16H 50/30

(57) ABSTRACT

Aspects of the disclosure are directed to mitigating ocular toxicity in patients undergoing treatment for B-cell disorders, including cancers. Such treatment may include administering a therapeutically effective dose of a component of a B-cell disorder therapy, monitoring for signs of ocular toxicity, and, if such signs are present, adjusting the dose of that component for a subsequent administration. Monitoring for signs of ocular toxicity may include performing an ophthalmic examination of the patient, including performing a visual assessment and corneal assessment of the patient. The ophthalmic examination may include selecting visual assessment options from predefined and selectable visual assessment options and include selection corneal assessment options from predefined and selectable corneal assessment options. An overall keratopathy visual acuity (KVA) grade may be determined based on the selected visual acuity assessment options and the selected corneal assessment options.

### CROSS-REFERENCE TO RELATED APPLICATION

This application claims priority to U.S. Patent Application No. 63/307,922 filed on Feb. 8, 2022, titled “Mitigating Ocular Toxicity,” and to U.S. Patent Application No. 63/323,127 filed on Mar. 24, 2022, titled “Mitigating Ocular Toxicity,” which are incorporated by reference herein in their entireties.

### TECHNICAL FIELD

This disclosure generally relates to mitigating ocular toxicity in patients being treated for B-cell disorders, including cancers.

### BACKGROUND

A variety of drugs and therapies have been evaluated and found effective in treating B-cell disorders, including cancers. Ocular toxicity is a known side effect in patients being treated with some of those drugs and therapies. Ocular toxicities are among the most common adverse events resulting from targeted anticancer agents and are becoming increasingly relevant in the management of patients on these agents. Currently, there remains a need for more effectively mitigating ocular toxicity in patients being treated for B-cell disorders, including cancers.

### SUMMARY

The following presents a simplified summary of various aspects described herein. This summary is not an extensive overview, and is not intended to identify key or critical elements or to delineate the scope of the claimed subject matter. The following summary merely presents some concepts in a simplified form as an introductory prelude to the more detailed description provided below. The disclosure encompasses corresponding apparatuses, systems, and computer-readable media.

Aspects of the disclosure are directed to mitigating ocular toxicity in patients undergoing treatment for B-cell disorders, including cancers. B-cell disorder therapies include cancer treatment therapies. Such treatment may include administering a therapeutically effective dose of a component of a B-cell disorder therapy, monitoring for signs of ocular toxicity, and, if such signs are present, adjusting the dose of that component for a subsequent administration. Monitoring for signs of ocular toxicity may include performing an ophthalmic examination of the patient, including performing a visual assessment and corneal assessment of the patient. The visual assessment may determine whether any changes have occurred to the patient's visual acuity. The corneal assessment may determine whether any adverse corneal events have occurred. An adverse corneal event may also be referred to as a keratopathy. A grade may be determined based on the ophthalmic examination, including the visual acuity assessment and the corneal assessment. This grade may represent both the visual acuity and corneal state of the patient. As such, this grade may be referred to as a keratopathy visual acuity (KVA) grade.

One aspect of the disclosure is a method of mitigating ocular toxicity. A therapeutically effective dose of a component of a B-cell disorder therapy is obtained. Instructions for administering the therapeutically effective dose to a patient are received. The instructions include an instruction to perform an ophthalmic examination of the patient during the B-cell disorder therapy. The therapeutically effective dose is administered to the patient in accordance with the instructions, and an ophthalmic examination of the patient is performed using a computer and in accordance with the instructions. The ophthalmic examination includes a visual acuity assessment and a corneal assessment of the patient. A keratopathy visual acuity grade may be obtained for the patient. To obtain the keratopathy visual acuity grade, at least one visual acuity assessment option indicating a visual acuity of the patient is selected from predefined and selectable visual acuity options presented at a user interface of the computing device, at least one corneal assessment option indicating a corneal state of the patient is selected from predefined and selectable corneal assessment options presented at the user interface, and the keratopathy visual acuity grade is automatically determined by the computing device based on the at least one visual acuity assessment option and the at least one corneal assessment option that were selected. A subsequent therapeutically effective dose of the B-cell disorder therapy is administered to the patient based on the keratopathy visual acuity grade obtained.

Another aspect of the disclosure is a computing device for mitigating ocular toxicity. The computing device includes one or more processors, a user interface, and a memory. The user interface includes predefined and selectable visual acuity assessment options and predefined and selectable corneal assessment options. The memory stores instructions that when executed by the one or more processors, cause the computing device to perform steps to obtain a keratopathy visual acuity grade for a patient that has received a therapeutically effective dose of a component of a B-cell disorder treatment. To obtain the keratopathy visual acuity grade, user input is received indicating selection of at least one visual acuity assessment option indicating a visual acuity of the patient from the predefined and selectable visual acuity assessment options, user input is received indicating selection of at least one corneal assessment option indicating a corneal state of the patient from the predefined and selectable corneal assessment options, and the keratopathy visual acuity grade is automatically determined based on the at least one visual acuity assessment option and the at least one corneal assessment option that were selected. The instructions, when executed by the one or more processors, also cause the computing device to present the keratopathy visual acuity grade at the user interface of the computing device.

Another aspect of the disclosure is another method for mitigating ocular toxicity. A therapeutically effective dose of a component of a B-cell disorder therapy is provided. Instructions for administering the therapeutically effective dose to a patient are also provided. The instructions include an instruction to perform one or more ophthalmic examinations of the patient during the B-cell disorder therapy. The instructions also include machine-readable data that, when read by a computing device, cause the computing device to navigate to a computer-executable application. The computer-executable application is configured to provide a keratopathy visual acuity grade based on a visual acuity assessment and a corneal assessment of an ophthalmic examination of the patient. The computer-executable application is configured to provide the keratopathy visual acuity grade by receiving user input indicating selection of at least one visual acuity assessment option indicating a visual acuity of the patient from predefined and selectable visual acuity assessment options presented at the user interface, receiving user input indicating selection of at least one corneal assessment options indicating a corneal state of the patient from predefined and selectable corneal assessment options presented at the user interface, and automatically determine the keratopathy visual acuity grade based on the at least one visual acuity assessment option and the at least one corneal assessment option selected. The computer-executable application is also configured to present the keratopathy visual acuity grade at the user interface of the computing device.

Another aspect of the disclosure is another method of mitigating ocular toxicity. A computing device may receive B-cell disorder treatment data and ophthalmic examination data. The B-cell disorder treatment data includes indication of one or more therapeutically effective doses of a component of a B-cell disorder therapy that have been administered to one or more patients. The ophthalmic examination data includes one or more visual acuity assessments and one or more corneal assessments of the patients. A computing device trains a machine learning model that is configured to provide one or more recommendations for administration of the component of the B-cell disorder therapy. The computing device trains the machine learning model based on the B-cell disorder treatment data and the ophthalmic examination data received. The computing device may receive additional B-cell disorder treatment data and additional ophthalmic examination data associated with a patient being treated with the B-cell disorder therapy. The additional B-cell disorder treatment data includes indication of one or more therapeutically effective doses of the component of the B-cell disorder therapy administered to the patient. The additional ophthalmic examination data includes one or more visual acuity assessments of the patient and one or more corneal assessments of the patient. The computing device uses the trained machine learning model to generate at least one recommendation for administration of the component of the B-cell disorder therapy to the patient. The computing device provides the at least one recommendation for administration of the component of the B-cell disorder therapy to a prescriber of the B-cell disorder therapy.

Another aspect of the disclosure is a computer-implemented method for mitigating ocular toxicity. A computing device displays, in association with an ophthalmic examination of a patient being treated for a B-cell disorder, a plurality of predefined and selectable visual acuity assessment options at a user interface of the computing device. The computing device displays, in association with the ophthalmic examination, a plurality of predefined and selectable corneal assessment options at the user interface. The computing device receives, at the user interface and in association with a visual acuity assessment of the ophthalmic examination, first user input indicating selection, from the plurality of predefined and selectable visual acuity assessment options, of at least one visual acuity assessment option that indicates a visual acuity of the patient. The computing device receives, at the user interface and in association with a corneal assessment of the ophthalmic examination, second user input indicating selection, from the plurality of predefined and selectable corneal assessment options, of at least one corneal assessment option that indicates a corneal state of the patient. The computing device determines, automatically based on the at least one visual acuity assessment option selected and based on the at least one corneal assessment option selected, a keratopathy visual acuity grade. The computing device causes output of the keratopathy visual acuity grade determined.

Another aspect of the disclosures is a system for mitigating ocular toxicity. The system may include a first computing device and a second computing device. The first computing device is configured to display, at a user interface of the computing device and in association with an ophthalmic examination of a patient being treated for a B-cell disorder, a plurality of predefined and selectable visual acuity assessment options. The first computing device is configured to display, at the user interface and in association with the ophthalmic examination, a plurality of predefined and selectable corneal assessment options. The first computing device is configured to receive, at the user interface and in association with a visual acuity assessment of the ophthalmic examination, first user input indicating selection, from the plurality of predefined and selectable visual acuity assessment options, of at least one visual acuity assessment option that indicates a visual acuity of the patient. The first computing device is configured to receive, at the user interface and in association with a corneal assessment of the ophthalmic examination, second user input indicating selection, from the plurality of predefined and selectable corneal assessment options, of at least one corneal assessment option that indicates a corneal state of the patient. The first computing device is configured to determine, automatically based on the at least one visual acuity assessment option selected and based on the at least one corneal assessment option selected, a keratopathy visual acuity grade. The first computing device is configured to send, to the second computing device, the keratopathy visual acuity grade determined. The second computing device is configured to receive, from the first computing device, the keratopathy visual acuity grade determined. The second computing device is configured to send, to the first computing device, at least one of a recommended dose of a component of a B-cell disorder therapy associated with the patient, or a recommended modification to the dose of the component of the B-cell disorder therapy.

Other systems, devices, and non-transitory computer-readable media that perform one or more of the methods steps described above are also contemplated and should be considered to be within the scope of the disclosures.

### BRIEF DESCRIPTION OF THE DRAWINGS

The present disclosures are described by way of example and not limited in the accompanying figures in which like reference numerals indicate similar elements and in which:

FIG. 1 is a block diagram of an example of a system for mitigating ocular toxicity in a patient being treated for a B-cell disorder, including cancer;

FIG. 2 is a block diagram of an example of a computing device that may be used to implement aspects of the disclosures;

FIG. 3 is a flowchart of example method steps for mitigating ocular toxicity in a patient being treated for a B-cell disorder, including cancer;

FIGS. 4A-4B are examples of screens of a graphical user interface for receiving user input indicating selection of visual acuity assessment options in accordance with some aspects of the disclosures;

FIGS. 5A-5B are examples of screens of a graphical user interface for receiving user input indicating selection of corneal assessment options in accordance with some aspects of the disclosures;

FIGS. 6A-6B are examples of alternative screens of a graphical user interface for receiving user input indicating selection corneal assessment options in accordance with some aspects of the disclosures;

FIG. 7 is an example of screens of a graphical user interface for presenting a keratopathy visual acuity grade in accordance with some aspects of the disclosures;

FIG. 8 is an example flowchart of example method steps for training a machine learning model for mitigating ocular toxicity and obtaining a dosing recommendation from the trained machine learning model in accordance with some aspects of the disclosures.

FIG. 9 is an example of a screen of another graphical user interface of an application for mitigating ocular toxicity in accordance with some aspects of the disclosures;

FIG. 10A is an example of screens of an alternative graphical user interface of an application for mitigating ocular toxicity in accordance with some aspects of the disclosures;

FIG. 10B is an example of screens of an alternative graphical user interface for receiving user input indicating selection of visual acuity assessment options in accordance with some aspects of the disclosures;

FIG. 10C-1 and FIG. 10C-2 is an example of screens of an alternative graphical user interface for receiving user input indicating selection of corneal assessment options in accordance with some aspects of the disclosures;

FIG. 10D is an example of screens of an alternative graphical user interface for receiving user input indicating selection of corneal assessment options and presenting a keratopathy visual acuity grade in accordance with some aspects of the disclosures;

FIG. 11A is an example of screens of another alternative graphical user interface of an application for mitigating ocular toxicity in accordance with some aspects of the disclosures;

FIG. 11B is an example of screens of another alternative graphical user interface for receiving user input indicating selection of visual acuity assessment options in accordance with some aspects of the disclosures;

FIG. 11C-1 and FIG. 11C-2 is an example of screens of another alternative graphical user interface for receiving user input indicating selection of corneal assessment options in accordance with some aspects of the disclosures;

FIG. 11D is an example of screens of another alternative graphical user interface for receiving user input indicating selection of corneal assessment options and presenting a keratopathy visual acuity grade in accordance with some aspects of the disclosures;

### DETAILED DESCRIPTION

The disclosures herein provide means for mitigating ocular toxicity in patients receiving B-cell disorder therapies. “B-cell disorders” include immunodeficiencies and disorders characterized by excessive or uncontrolled proliferation, such as acute glomerulonephritis, chronic lymphocytic leukemia (CLL), diffuse large B-cell lymphoma (DLBCL), epidermolysis bullosa acquisita, extramedullary), follicular lymphoma (FL), Goodpasture's syndrome, heavy chain disease, Hodgkin's lymphoma (HL), idiopathic thrombocytopeniaurpura (ITP), light chain deposition disease, lymphoplasmacytic lymphoma (LPL), monoclonal gammopathy of undetermined significance (MGU.S.), multiple myeloma (MM), non-Hodgkin's lymphoma B-cell leukemia (NHL), non-secretory multiple myeloma, pemphigus and pemphigoid disorders, plasma cell leukemia, POEMS syndrome/osteosclerotic myeloma, primary amyloidosis (AL), smoldering multiple myeloma, solitary plasmacytoma (bone, systemic lupus erythematosus SLE), Type I and II cryoglobulinemia, and Waldenström's macroglobulinemia. For example, the B-cell disorder may be a leukemia (e.g., chronic myelocytic leukemia, acute myelocytic leukemia, chronic lymphocytic leukemia, acute lymphocytic leukemia), a lymphoma (e.g., non-Hodgkin's lymphoma or Hodgkin's lymphoma), or a plasma cell malignancy (e.g., multiple myeloma, plasma cell myeloma, plasmablastic lymphoma, anaplastic lymphoma kinase positive large B-cell lymphoma). B-cell disorder therapies can be used to treat B-cell mediated disorders, including cancers, in a patient need of such treatment. “Treating” as used herein refers to alleviating one or more symptoms or effects associated with a disorder and/or slowing the progression of the disorder.

Administering therapeutically effective doses of components of B-cell disorder therapies are known to sometimes cause ocular toxicity is some patients. For example, as described in U.S. Provisional Patent Application No. 62/558,593 titled “Combination Therapy for B-Cell Disorders,” incorporated by reference herein in its entirety, belantamab mafodotin (e.g., BLENREP®) is one such component of a B-cell disorder therapy. Additional dosing regimens for belantamab mafodotin are provided herein and described in U.S. Provisional Patent Application No. 63/302,924 titled “Combination Therapy for Cancer,” incorporated by reference herein in its entirety. The current U.S. Prescribing Information for BLENREP® indicates that ocular toxicity is a known side effect at the recommended dosages. “Ocular toxicity” refers to any unintended exposure of a therapeutic agent to ocular tissue. Ocular toxicity can include changes in corneal epithelium, dry eyes, irritation, redness, blurred vision, photophobia, corneal ulcers, and/or changes in visual acuity. Other examples of targeted cancer treatment therapies that have been associated with ocular toxicity issues include those shown in Table 1 below:

TABLE 1
Target Therapies and Ocular Toxicity Issues

| Therapy | Ocular Toxicity Issues |
|---|---|
| Afatinib | Conjunctivitis 11% n = 229 Keratitis 0.8% n = 3865 patients with 1 grade III event. |
| (Gilotrif) | |
| Bortezomib | Label mentions diplopia, blurred vision, conjunctival infection, irritation. |
| (Velcade) | Case reports associated with eyelid chalazia. |
| Ceritinib | Vision disorder 9% (comprised of vision impairment, blurred vision, photopsia, |
| (Zykadia) | accommodation disorder, presbyopia, or reduced visual acuity) n = 255 |
| Crizotinib | Vision disorder 64% (Includes diplopia, photopsia, photophobia, vision blurred, |
| (Xalkori) | visual field defect, visual impairment, vitreous floaters, visual brightness, and |
| | visual acuity reduced.) n = 255 |
| Dabrafenib | Uveitis/Iritis 1% n = 586. Can cause cystoid macula edema. |
| (Tafinlar) | |
| Dasatinib | Visual disorder (visual disturbance, vision blurred, decreased visual acuity) |
| (Sprycel) | or dry eye in 1-10% Conjunctivitis in 0.1-1% |
| Erlotinib | Reported Conjunctivitis in 18% n = 84, Conjunctivitis 12% (with 1 Grade III) |
| (Tarceva) | n = 485 and Keratoconjunctivitis Sicca 12% n = 485. Mentions of corneal |
| | perforation or ulceration, and abnormal eyelash growth. The pooled incidence of |
| | ocular disorders was 17.8% in three lung cancer studies and 12.8% in one |
| | pancreatic cancer study. |
| Gefitinib | Mentions of eye irritation, eye pain, corneal erosion/ulcer, aberrant eyelash |
| (Iressa) | growth, corneal membrane sloughing, and ocular ischemia/hemorrhage. |
| Imatinib | Estimated 1%-10%: conjunctivitis, vision blurred, eyelid edema, conjunctival |
| (Gleevec) | hemorrhage, dry eye Estimated 0.1%-1%: eye irritation, eye pain, orbital edema, |
| | scleral hemorrhage, retinal hemorrhage, blepharitis, macular edema. Estimated |
| | 0.01%-0.1%: papilledema, glaucoma, cataract. Among FDA cited studies |
| | (n = 729), incidence of periorbital edema was 57.8%, hyperlacrimation 14.1%, |
| | and visual disturbance 7.1%. |
| Nilotinib | Common: eye hemorrhage, periorbital edema, eye pruritus, conjunctivitis, dry eye. |
| (Tasigna) | Uncommon: vision impairment, vision blurred, visual acuity reduced, photopsia, |
| | eye irritation. Unknown frequency: papilloedema, diplopia, photophobia, eye |
| | swelling, blepharitis, eye pain, chorioretinopathy, conjunctival hemorrhage, |
| | conjunctivitis allergic, conjunctival hyperaemia, ocular hyperaemia, ocular surface |
| | disease, scleral hyperaemia. |
| Trametinib | Mentions blurry vision, dry eye, transient blindness, eye floaters, and visual |
| (Mekinist) | halo. Incidence of Retinal Veinous Occlusion was 0.2% (4/1,749). |
| | Incidence of Retinal Pigment Epithelial Detachment was 0.8% (14/1,749). |
| Vandetanib | Blurry vision (corneal opacities) 9%. No cohort information. |
| (Caprelsa) | |
| Vemurafenib | Mentions retinal vein occlusion, uveitis, blurry vision, iritis, and photophobia |
| (Zelboraf) | |
| Cetuximab | Label mentions existence of blepharitis, conjunctivitis, keratitis/ulcerative |
| (Erbitux) | keratitis with decreased visual acuity, and cheilitis. |
| Ipilimumab | Label mentions existence of uveitis, iritis, episcleritis, conjunctivitis, and |
| (Vervoy) | blepharitis. |
| Panitimumab | Eye/eyelid irritation (1%), conjunctivitis (4%), ocular hyperemia (3%), |
| (Vectibix) | increased lacrimation (2%), in 463 patients |
| Pertuzumab | Mentions increased lacrimation |
| (Perjeta) | |
| Rituximab | Mentions uveitis and optic neuritis |
| (Rituxan, | |
| Mabthera) | |

The B-cell disorder therapy may be a combination therapy. “Combination therapy” means a therapy comprising two or more active agents. The two or more active agents can be administered in one dosage form or in separate dosage forms. The two or more active agents can be administered at the same time (in one or more dosage forms) or at separate times and can each be administered by the same route or by different routes. Examples of components of B-cell disorder therapies include an anti-B-cell maturation antigen (BMCA) binding protein, such as belantamab mafodotin (e.g., BLENREP®), and are described in U.S. Provisional Patent Application No. 62/558,593. A B-cell disorder therapy may include a cytotoxic agent, e.g., an auristatin such as monomethyl auristatin E (MMAE) or monomethyl auristatin F (MMAF). A B-cell disorder therapy may include an antibody drug conjugate.

The disclosures herein may be employed to mitigate ocular toxicity arising from other components of B-cell disorder therapies, from other types of therapies, or from other drug treatments.

Risk evaluation and mitigation strategies may be employed to monitor for ocular toxicity and, if detected, adjust the respective doses of one or more components of a B-cell disorder therapy. To detect ocular toxicity, an ophthalmic examination of a patient may be performed. The ophthalmic examination may include a visual acuity assessment of the patient and a corneal assessment of the patient. The visual acuity assessment of the patient may determine whether any changes to the patient's visual acuity have occurred. The changes to the patient's visual acuity may be changes to the patient's best corrected visual acuity (BCVA). The corneal assessment may determine whether any keratopathies (adverse corneal events) have occurred. The ophthalmic examination may be performed prior to each administration of a dose of the component of the B-cell disorder therapy. An ophthalmic examination may also include one or more of: documentation of manifest refraction and the method used to obtain best corrected visual acuity, intraocular pressure measurement, anterior segment (slit lamp) examination including fluorescein staining of the cornea and lens examination, dilated funduscopic examination, an ocular surface disease index (OSDI) which is visual function questionnaire that assess the impact of potential ocular change in vision on function and health-related quality of life. Ophthalmic examination data may include indication of one or more of these evaluations in various combinations.

Depending on the ophthalmic examination results, the same dose may be administered to the patient, a lower dose may be administered to the patient, or treatment may be withheld until the patient's visual acuity and corneal state improve then continue at the same dose or a reduced (lower) dose. The dose subsequently administered to the patient may be determined by the prescriber of the B-cell disorder therapy with or without the input of a medical monitor (e.g., a hematologist, an oncologist, etc.) The medical monitor may assist the prescriber in determining a recommended modification to the dose of the component of the B-cell disorder therapy based on the patient's medical history and any ophthalmic examinations performed during the course of treatment. As described in further detail below, machine learning and artificial intelligence may be employed to provide recommended doses or modifications to current doses.

The disclosures herein can result in faster and improved dosing decisions for patients being treated for B-cell disorders, including cancer. Those improved dosing decisions can, in turn, result in better patient outcomes in terms of both treating the underlying B-cell disorder and mitigating any ocular toxicity arising as a side effect from a component of the B-cell disorder therapy. The disclosures herein provide, for example, improvements to the process of conducting ophthalmic examinations of patients being treated with B-cell disorder therapies having known ocular toxicity side effects. Those improvements include faster receipt of the ophthalmic examination data used to make dosing decisions, faster delivery of the ophthalmic examination data to medical providers that provide input on dosing decisions, faster receipt of feedback from medical providers regarding recommended dosing decisions, and reducing opportunities for potential human error when grading eye exams (e.g., when determining a decrease in visual acuity, calculating visual acuity grades, corneal as grades, or KVA grades, etc.). The following scenario illustrates some of the improvements provided by the present disclosures. A patient being treated for a B-cell disorder may have received a therapeutically effective dose of a component of a B-cell disorder therapy. Prior to receiving the next dose, the patient may undergo an ophthalmic examination by an ophthalmologist in order to determine the presence of any ocular toxicity that might warrant an adjustment to the dose administered to the patient. During the ophthalmic examination (e.g., in real-time during the examination), the ophthalmologist may use a computer-executable application to select from various predefined and selectable visual acuity assessment options to indicate the visual acuity of the patient. Similarly, during the ophthalmic examination (e.g., in real-time), the ophthalmologist may use the computer-executable application to select from various predefined and selectable corneal assessment options to indicate the corneal state of the patient. Based on the visual acuity assessment options and the corneal assessment options selected, the computer-executable application may automatically determine (e.g., in real time) a KVA grade for the patient. The KVA grade may map to a corresponding dosing decision. The computer-executable application may also share (e.g., in real-time) the ophthalmic examination data and any relevant patient data with another medical service provider for review. That medical service may then provide (e.g., in real-time) one or more recommendations regarding dosing decisions for the next dose of the component of the B-cell disorder therapy. As such, in some examples, a patient may receive real-time (or near real-time) feedback throughout the course of treatment regarding dosing decisions during or in close temporal proximity to any ophthalmic examinations that are recommended (or required) prior to each administration of the component of the B-cell disorder therapy. This ensures that patients and medical providers (e.g., administrators of the B-cell disorder therapies) may receive timely feedback about dosing decisions.

A B-cell disorder therapy may be provided as a kit-of-parts. The kit-of-parts may include one or more pharmaceutical compositions together with instructions for use. For convenience, the kit-of-parts may comprise reagents in predetermined amounts with instructions for use.

A kit may include a plurality of syringes, ampules, foil packets, or blister packs, each containing a single unit dose of a kit component described herein. Containers of a kit may be airtight, waterproof (e.g., impermeable to changes in moisture or evaporation), and/or light-tight. A kit may include a device suitable for administration of the component, e.g., a syringe, inhalant, pipette, forceps, measured spoon, dropper (e.g., eye dropper), swab (e.g., a cotton swab or wooden swab), or any such delivery device. In some examples, the device may be a medical implant device, e.g., packaged for surgical insertion. A kit disclosed herein may comprise one or more reagents or instruments which enable the method to be carried out.

A kit may also include instructions for using the B-cell disorder therapy. These instructions may be present in the kit in a variety of forms, such as printed information on a suitable medium or substrate (e.g., a piece or pieces of paper on which the information is printed), in the packaging of the kit, in a package insert, etc. The instructions for use may additionally or alternatively be provided on a computer-readable medium (e.g., disk, jump/thumb drive, CD, etc.), on which the information has been recorded or at a website address which may be used via the Internet to access the information at the website.

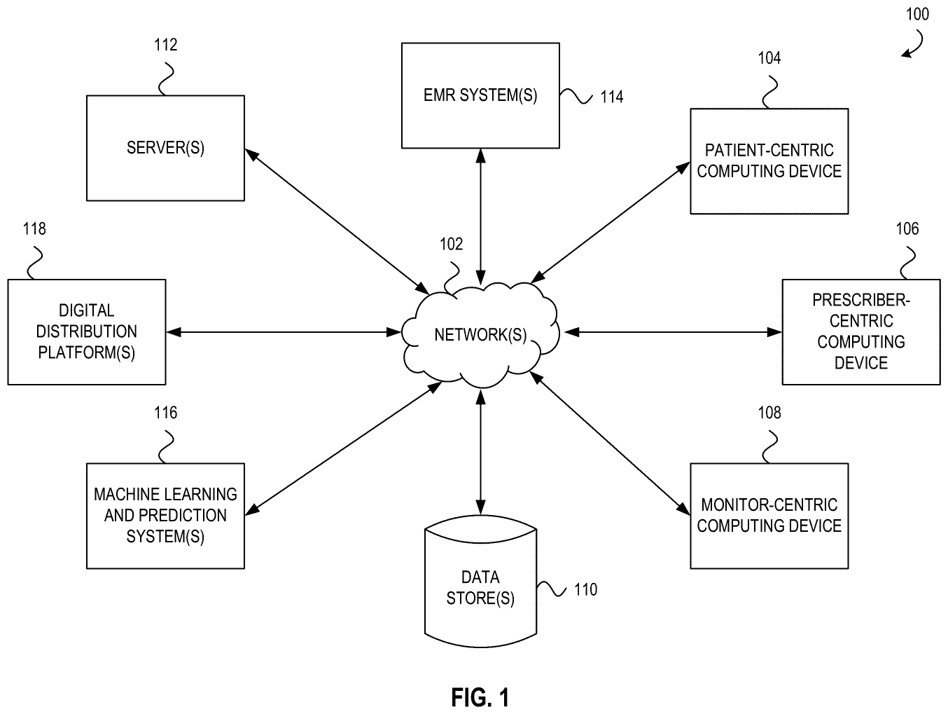

Turning now to FIG. 1, a block diagram of an example of a system 100 for mitigating ocular toxicity in a patient being treated for a B-cell disorder, including cancer, is shown. The system 100 may include various computing device in signal communication with one another via one or more networks 102. The network(s) 102 may include one or more public networks or private networks, one or more local area networks (LANs), one or more wide area networks (WANs) such as the Internet, one or more cellular networks, and the like. The network(s) 102 may include wired networks and wireless networks.

The system 100 shown by way of example in FIG. 1 includes multiple computing devices 104, 106, and 108 configured to mitigate ocular toxicity in patients being treated for B-cell disorders, including cancers. The computing devices 104, 106, and 108 may be operated respectively by each of the patient being treated for a B-cell disorder, the prescriber administering the B-cell disorder to the therapy, and a medical monitor that monitors the administration of the B-cell disorder therapy to the patient throughout the duration of the treatment. As such, and for convenience, the computing devices 104, 106, and 108 may be referred to as a patient-centric computing device 104, a prescriber-centric computing device 106, and a monitor-centric computing device 108. The system 100 may also include one or more data stores 110 (e.g., databases or other data repositories), one or more servers 112 (e.g., web servers, application servers, etc.), one or more electronic medical record (EMR) systems 114 (electronic health record, (EHR) systems), one or more machine learning and prediction systems 116, and one or more digital distribution platforms 118 (e.g., application (“app”) store, application marketplace, etc.). In some examples, and as described in further detail below, the system may be accessed, e.g., via an ocular toxicity mitigation application, using a code (e.g., a QR code or the like) included on the prescribing information for the B-cell disorder therapy.

Each computing device 104, 106, and 108, may be configured with or capable of accessing an ocular toxicity mitigation application. The functionality of the ocular toxicity mitigation application may depend on which type of user (e.g., patient, prescriber, monitor) that is intended to operate it. As such, and again for convenience, the various ocular toxicity mitigation applications may be referred to as a patient-centric ocular toxicity mitigation application, a prescriber-centric ocular toxicity mitigation application, and a monitor-centric ocular toxicity mitigation application. The ocular toxicity mitigation application(s) may be in signal communication with each other via the network(s) 102.

The patient-centric ocular toxicity mitigation application at the patient-centric computing device 104 may be configured to assist a patient during treatment of a B-cell disorder therapy. For example, the patient-centric ocular toxicity mitigation application may be configured to receive and store user input indicating patient-reported outcomes related to the administration of the B-cell disorder therapy. Those patient-reported outcomes may be included in the ophthalmic examination data to guide the ocular toxicity grading and determination of a subsequent dose of a component of a B-cell disorder therapy. The patient-centric ocular toxicity mitigation application may be configured to output alerts (e.g., reminders) of upcoming ophthalmic examinations that need to be performed before the next dose of the component of the B-cell disorder therapy can be administered. Alerts may also include other types of reminders such as reminders of upcoming appointments (e.g., for an ophthalmic examination, for an dosage administration, etc.), reminders to schedule subsequent appointments, reminders to take medication at appropriate times, and the like. The patient-centric ocular toxicity mitigation application may also be configured to receive (e.g., via a push message) and output alerts, questionnaires, and the like. The questionnaires may include questions about the patient's experience with the B-cell disorder therapy. The patient may answer the questionnaire via a user interface of the patient-centric ocular toxicity mitigation application, and the patient-centric ocular toxicity mitigation application may be configured to provide the patient's answers to, e.g., the prescriber of the B-cell disorder therapy, a medical monitor of the administration of the B-cell disorder therapy to the patient, a manufacturer of one or more components of the B-cell disorder therapy, and the like. In this way, use of the patient-centric computing device 104 and patient-centric ocular toxicity mitigation application advantageously can lead to better patient outcomes through consistent and timely administration of the B-cell disorder therapy and improved dosing decisions throughout the duration of treatment. The patient-centric computing device 104 and patient-centric ocular toxicity mitigation application may be configured to provide a portal, dashboard, platform, or the like for accessing the ophthalmic examination data and other treatment data compiled during the course of treatment. The patient-centric computing device 104 and patient-centric ocular toxicity mitigation application may be configured transfer that ophthalmic examination and treatment to a new prescriber or other health care provider.

The patient-centric ocular toxicity mitigation application may be in signal communication with the one or more EMR systems 114 to, e.g., obtain and present medical information included in the patient's electronic medical records. The patient-centric ocular toxicity mitigation application may be in signal communication with one or more data stores 110 to, e.g., obtain and present ophthalmic examination data and treatment data associated with the patient. The patient-centric computing device 104 may be in signal communication with one or more of the servers 112 or the digital distribution platforms 118 to access or otherwise obtain the patient-centric ocular toxicity mitigation application.

The prescriber-centric ocular toxicity mitigation application at the prescriber-centric computing device 106 may be configured to assist a prescriber of the B-cell disorder therapy administer doses of a component of the B-cell disorder therapy throughout the treatment of a patient. For example, and as described in further detail below, the prescriber-centric ocular toxicity mitigation application may be used to perform the ophthalmic examination of the patient prior to a subsequent dose of a component of a B-cell therapy. The therapy may be administered by the prescriber itself or, additionally or alternatively, a medical provider associated with or acting at the direction of the prescriber. The prescriber-centric ocular toxicity mitigation application may be configured to provide an easily operated and intuitive user interface for receiving input from the prescriber regarding a visual acuity assessment and corneal assessment performed during the ophthalmic examination. The prescriber-centric ocular toxicity mitigation application may also be configured to receive visual acuity assessments in one or more visual acuity units including Snellen (feet), Snellen (metric), MAR (Minimum Angle of Resolution), logMAR (Logarithm of the Minimum Angle of Resolution, also referred to as Log Mar or Log MAR), decimal, and other systems for representing visual acuity. The prescriber-centric ocular toxicity mitigation application may thus be configured to convert between different visual acuity units depending on the preference of the prescriber.

The prescriber-centric ocular toxicity mitigation application also may be configured to automatically determine changes to a patient's baseline BCVA. The patient's baseline BCVA may be obtained prior to an initial administration of the component of a B-cell disorder therapy. Dosing decisions for subsequent administrations of that component may be based on a number of lines of decrease from the patient's baseline BCVA. A decrease in a patient's baseline BCVA may be indicated by one or more lines of decrease in visual acuity assessment. For example, if a patient's baseline BCVA is determined to be 20/20 (Snellen units), a one line decrease in the baseline BCVA may be a 20/25 visual acuity assessment; a two to three line decrease in the baseline BCVA may be a 20/30 to 20/40 visual acuity assessment; a more than three line decrease in the baseline BCVA may be a 20/50 to 20/200 visual acuity assessment. Additional examples of various lines of decrease in baseline BCVA are provided in Table 2 and Table 3 below. Other systems for assessing changes to lines of vision may be employed, e.g., any suitable version of the Common Terminology Criteria for Adverse Events (CTCAE) that define grades for decreases in visual acuity.

TABLE 2
Example Guidance for Assessing Visual Acuity.

| | | | >3 lines | |
|---|---|---|---|---|
| Baseline Best | | | decrease from | |
| Corrected | 1 line decrease | 2-3 lines | Baseline BCVA | |
| Visual Acuity | from Baseline | decrease from | but not worse | BCVA worse |
| (BCVA) | BCVA | Baseline BCVA | than 20/200 | than 20/200 |
| 20/15 to 20/16 | 20/20 | 20/25 to 20/30 | 20/40 to 20/200 | Worse than |
| | | or 20/32 | | 20/200 |
| 20/20 | 20/25 | 20/30 to 20/40 | 20/50 to 20/200 | Worse than |
| | | | | 20/200 |
| 20/25 | 20/30 or 20/32 | 20/40 to 20/50 | 20/60 to 20/200 | Worse than |
| | | | | 20/200 |
| 20/30 or 20/32 | 20/40 | 20/50 to 20/60 | 20/70 to 20/200 | Worse than |
| | | or 20/63 | | 20/200 |

TABLE 3
Additional Example Guidance for Assessing Visual Acuity.

| | | | >3 lines | |
|---|---|---|---|---|
| Baseline Best | | | decrease from | |
| Corrected | 1 line decrease | 2-3 lines | Baseline BCVA | |
| Visual Acuity | from Baseline | decrease from | but not worse | BCVA worse |
| (BCVA) | BCVA | Baseline BCVA | than 20/200 | than 20/200 |
| 20/15 to 20/16 | 20/20 | 20/25 to 20/30 | 20/40 to 20/200 | Worse than |
| | | or 20/32 | | 20/200 |
| 20/20 | 20/25 | 20/30 to 20/40 | 20/50 to 20/200 | Worse than |
| | | | | 20/200 |
| 20/25 | 20/30 or 20/32 | 20/40 to 20/50 | 20/60 to 20/200 | Worse than |
| | | | | 20/200 |
| 20/30 or 20/32 | 20/40 | 20/50 to 20/60 | 20/70 to 20/200 | Worse than |
| | | or 20/63 | | 20/200 |
| 20/40/ | 20/50 | 20/60 to 20/70 | 20/100 to 20/200 | Worse than |
| | | or 20/80 | | 20/200 |
| 20/50 | 20/60 or 20/63 | 20/70 to 20/100 | 20/125 to 20/200 | Worse than |
| | | | | 20/200 |
| 20/60 or 20/63 | 20/70 or 20/80 | 20/100 to 20/125 | 20/150 to 20/200 | Worse than |
| | | | | 20/200 |
| 20/70 or 20/80 | 20/100 | 20/125 to 20/150 | 20/200 | Worse than |
| | | or 20/160 | | 20/200 |
| 20/100 | 20/125 | 20/150 to 20/200 | N/A | Worse than |
| | | | | 20/200 |
| 20/125 | 20/150 or | 20/200 | N/A | Worse than |
| | 20/160 | | | 20/200 |
| 20/150 or 20/160 | 20/200 | N/A | N/A | Worse than |
| | | | | 20/200 |
| Worse than | N/A | N/A | N/A | Any further |
| 20/160 | | | | reduction from |
| | | | | baseline |

Appropriate dosing decisions depend on accurate assessments of changes to baseline BCVA. Erroneous determinations of changes to lines of vision can lead to inaccurate dosing decisions. The prescriber-centric ocular toxicity mitigation application may be configured to provide predefined visual acuity assessment options the prescriber can select from and automatically calculate changes to lines of vision based on the visual acuity assessment options the prescriber selects. In this way, the prescriber-centric ocular toxicity mitigation application advantageously reduces the likelihood of any errors in calculating changes to the lines of vision which, in turn, reduces the likelihood of erroneous visual acuity grading and erroneous dosing decisions thus improving patient outcomes during the course of treatment with the B-cell disorder therapy. Patient safety may be improved by ensuring the patient receives the appropriate dose of the component of the B-cell disorder therapy at the appropriate time which can contribute to a decrease in adverse corneal events. The prescriber-centric ocular toxicity mitigation application may also be configured to automatically calculate, for both of the patient's left eye and right eye, a respective visual acuity grade based on the selected visual acuity assessment options and the determined changes to the lines of vison. The prescriber-centric ocular toxicity mitigation application may further be configured to use the visual acuity grades to calculate an overall KVA grade for the patient. Calculating a visual acuity grade may include, for example, mapping a change in visual acuity to a numerical score (e.g., 1-10, 1-100, etc.), letter grade (e.g., A-Z), and the like.

The prescriber-centric ocular toxicity mitigation application may also be configured to assist the prescriber in performing a corneal assessment of the patient during the ophthalmic examination. The corneal assessment may include an evaluation of the state of the patient's cornea to determine the presence of any keratopathies (corneal diseases). The prescriber-centric ocular toxicity mitigation application may be configured to provide predefined corneal assessment options the prescriber can select from. In this way, the prescriber-centric ocular toxicity mitigation application advantageously provides a means for quickly and intuitively indicating the corneal state of the patient. The prescriber-centric ocular toxicity mitigation application may also be configured to automatically calculate, for both of the patient's left eye and right eye, a respective corneal grade based on the selected corneal assessment options. The prescriber-centric ocular toxicity mitigation application may further be configured to use the corneal grades to calculate an overall KVA grade for the patient. Calculating a corneal grade may include, for example, mapping a selected corneal assessment option to a numerical score (e.g., 1-10, 1-100, etc.), letter grade (e.g., A-Z), and the like. Calculating the corneal grade may include, for example, mapping each corneal assessment option to a numerical score to obtain a set of numerical scores corresponding to the selected corneal assessment options. Calculating the corneal grade may include calculating a sum, an average, a weighted average, and the like for the set of numerical scores.

The KVA grade may be based on the visual acuity assessment and the corneal assessment of the patient. In some examples, the KVA grade may be one of four grades (e.g., “Grade 1,” “Grade 2,” “Grade 3,” or “Grade 4”). As noted above, the visual acuity assessment may characterize or otherwise represent any decline from the patient's baseline BCVA. The corneal assessment may characterize or otherwise represent the corneal state of the patient including a mild, moderate, or severe superficial keratopathy as well as any corneal epithelial defect. A mild superficial keratopathy may include a mild superficial punctate keratopathy with a documented decrease in baseline BCVA, with or without symptoms. A moderate superficial keratopathy may include any or a combination of a moderate superficial punctuate keratopathy, patchy microcyst-like deposits, sub-epithelial haze (peripheral), or any new peripheral stromal opacity. A severe superficial keratopathy may include any or a combination of severe superficial punctuate keratopathy, diffuse microcyst-like deposits involving the central cornea, sub-epithelial haze (central), or any new stromal opacity. A corneal epithelial defect includes an epithelial defect with underlying stromal infiltration such as a corneal ulcer. An example KVA grading scale for treatment-related corneal events is shown in Table 4 below.

TABLE 4
Example KVA Grading Scale.

| Grade per KVA Scale | | Grade 1 | Grade 2 | Grade 3 | Grade 4 |
|---|---|---|---|---|---|
| Corneal | Corneal | Mild | Moderate | Sever | Corneal |
| Toxicities | Assessment | superficial | superficial | superficial | epithelial defect |
| | | keratopathy | keratopathy | keratopathy | |
| | Visual | 1 line decrease | 2-3 line decrease | More than 3 | Visual acuity |
| | Acuity | from baseline | from baseline | line decrease | worse than |
| | Assessment | BCVA (Snellen) | BCVA and | from baseline | 20/200 (Snellen) |
| | | | visual acuity | BCVA and | |
| | | | not worse than | visual acuity | |
| | | | 20/200 (Snellen) | not worse than | |
| | | | | 20/200 (Snellen) | |

The prescriber-centric ocular toxicity mitigation application may include or otherwise be configured to use a KVA grading scale such as the one shown in Table 4 above in order to automatically determine the overall KVA grade for the patient (e.g., Grade 1-4). The overall KVA grade determined for the patient may be used to determine an appropriate dosage modification for a subsequent administration of a component of the B-cell disorder therapy. Example dosage modifications are shown in Table 5 below.

TABLE 5
Example Dosage Modifications.

| KVA Grade | Recommended Dosage Modification |
|---|---|
| Grade 1 | Continue treatment at current dose. |
| Grade 2 | Withhold component of B-cell disorder |
| | therapy until improvement in both corneal |
| | assessment and visual acuity assessment, and |
| | resume at same dose. |
| Grade 3 | Withhold component of B-cell disorder |
| | therapy until improvement in both corneal |
| | assessment and visual acuity assessment, and |
| | resume at reduced dose. |
| Grade 4 | Permanent discontinuation of component of |
| | B-cell disorder therapy or withhold |
| | component of B-cell disorder therapy until |
| | improvement in both corneal assessment and |
| | visual acuity assessment, and resume at |
| | reduced dose. |

The prescriber-centric ocular toxicity mitigation application may include or otherwise be configured to use a dosage modification table such as the one show in Table 5 above in order to automatically output a recommended dosage modification to the prescriber. In the event a dosage reduction is recommended, the prescriber-centric ocular toxicity mitigation application may further be configured to provide a recommended dose for a subsequent administration of the component of the B-cell disorder therapy to the patient. As described in further detail below, the prescriber-centric ocular toxicity mitigation application may be configured to determine or otherwise provide the recommended dose based on one or more recommended doses received from a medical monitor that monitors the prescriber's administration of the B-cell disorder therapy to the patient, one or more recommended doses determined using machine learning and artificial intelligence, or a combination of both. The prescriber-centric ocular toxicity mitigation application may be configured to output the recommended dosage modification(s) and recommended dose(s) by, e.g., presenting them at a user interface presented at a display of the prescriber-centric computing device 106. In this way, the prescriber-centric ocular toxicity mitigation application can contribute to consistent management of corneal events and consistent dosing decisions across the patient population.

The prescriber-centric ocular toxicity mitigation application may also be configured with other features that assist the prescriber in treating the patient with the B-cell disorder therapy. The prescriber-centric ocular toxicity mitigation application may be configured to retrieve the patient's baseline BCVA which may be stored in an electronic medical record or other data store associated with the patient thus avoiding the need to input the baseline BCVA during each ophthalmic examination. The prescriber-centric ocular toxicity mitigation application may be configured to store (e.g., in a local or remote data store) prescriber notes and ophthalmic examination data obtained during the ophthalmic examination(s) of the patient(s), e.g., visual acuity assessments, corneal assessments including tracking of adverse corneal events. The prescriber-centric ocular toxicity mitigation application may be configured to compare previous ophthalmic examinations of a patient based on the stored ophthalmic examination data. For example, the prescriber-centric ocular toxicity mitigation application may be configured to track a patient's visual acuity assessments and corneal assessments over time. As another example, the prescriber-centric ocular toxicity mitigation application may map corneal events and visual acuity over time based on the stored ophthalmic examination data. The prescriber-centric ocular toxicity mitigation application may be in signal communication with or otherwise integrate with a patient's electronic medical record(s) to include ophthalmic examination data, dosing decisions, and the like. The prescriber-centric ocular toxicity mitigation application may be configured to automatically provide guidance based on the determined KVA grades, e.g., increasing the frequency of ophthalmic examinations, provide information about the benefits and risks associated with various dosing decisions, and provide other suggestions for managing the administration of the B-cell disorder therapy. The prescriber-centric ocular toxicity mitigation application may also be configured to receive and output patient-reported information regarding the patient's experience with the B-cell disorder therapy, e.g., answers to questions provided in a questionnaire presented to the patient.

The prescriber-centric ocular toxicity mitigation application may be in signal communication with the one or more EMR systems 114 to, e.g., obtain and provide medical information included in a patient's electronic medical records. The prescriber-centric ocular toxicity mitigation application may be in signal communication with one or more data stores 110 to, e.g., obtain and store ophthalmic examination data and treatment data associated with the patient. The prescriber-centric computing device 104 may be in signal communication with one or more of the servers 112 or the digital distribution platforms 118 to access or otherwise obtain the prescriber-centric ocular toxicity mitigation application. The prescriber-centric ocular toxicity mitigation application may be in signal communication with the machine learning and prediction systems 116 to provide ophthalmic examination data, treatment data, and patient data and to obtain one or more recommended doses of a component of the B-cell disorder therapy that have been determined using machine learning and artificial intelligence. Inputting all of the visual acuity assessment data and corneal assessment data may trigger the prescriber-centric ocular toxicity mitigation application to automatically submit a request to a medical monitor to provide one or more recommended dosing modifications and one or more recommended doses of a component of a B-cell disorder therapy. Inputting all of the visual acuity assessment data and corneal assessment data may also trigger the prescriber-centric ocular toxicity mitigation application to automatically obtain one or more recommended dosage modifications and one or more recommended doses from the machine learning and prediction system(s) 116.

The monitor-centric ocular toxicity mitigation application at the prescriber-centric computing device 106 may be configured to assist a prescriber of a B-cell disorder therapy with evaluating ophthalmic examination data, determining recommended dosage modifications, and determining recommended doses of the component(s) of B-cell disorder therapies being used to treat patients with B-cell disorders. For example, the monitor-centric ocular toxicity mitigation application may be in signal communication with the prescriber-centric ocular toxicity mitigation application at the prescriber-centric computing device 106. The monitor-centric ocular toxicity mitigation application may receive ophthalmic examination data, treatment data, patient data, prescriber notes, and the like from the prescriber-centric ocular toxicity mitigation application and send the recommended dosing modification(s) and recommended dose(s) to the prescriber-centric ocular toxicity mitigation application. The monitor-centric ocular toxicity mitigation application may additionally or alternatively obtain some or all of such data and information from a remote data store that has received it from the prescriber-centric ocular toxicity mitigation application. The monitor-centric ocular toxicity mitigation application may also obtain this data and information from an electronic medical record associated with the patient. As an example, the prescriber-centric ocular toxicity mitigation application may be configured to provide or otherwise cause the monitor-centric ocular toxicity mitigation application to obtain one or more of the ophthalmic examination data, visual acuity assessment data, corneal assessment data, visual acuity grade(s), corneal grade(s), or KVA grade. The medical monitor may review this information and determine a recommended dosage modification (e.g., no change, reduced dose, etc.) as well as a recommended dose.

The monitor-centric ocular toxicity mitigation application may be configured to output alerts when a prescriber needs to make a dosing decision for a patient. Upon receiving the alert, the medical monitor may review and evaluate the ophthalmic examination data, patient data, prescriber notes, and the like in order to determine one or more recommended dosing modifications and one or more recommended doses to be administered to the patient. As described in further detail below, the monitor-centric ocular toxicity mitigation application may be configured to use machine learning and artificial intelligence to determine the recommended dosing modification(s) and recommended dose(s). The monitor-centric ocular toxicity mitigation application may send to the prescriber or otherwise make available for the prescriber the recommended dosing modification(s) and recommended dose(s). By alerting a medical monitor that a dosing decision is needed for a patient, the monitor-centric ocular toxicity mitigation application can facilitate real-time or near real-time dosing decisions during an ophthalmic examination of a patient (e.g., after the ophthalmic examination has concluded and before a final dosing decision is made, during a medical consultation with the patient, etc.). By improving the speed of arriving at an appropriate dosing decision, the monitor-centric ocular toxicity mitigation application advantageously ensures a patient can receive an appropriate dose of a component of a B-cell disorder therapy at the appropriate time thus again improving patient outcomes including mitigation of ocular toxicity, reduction of adverse corneal events, and the like.

In some examples, the system 100 may include a mediator to facilitate the collaboration between the medical monitors that use the monitor-centric ocular toxicity mitigation application and the prescribers that use the prescriber-centric ocular toxicity mitigation application. For example, a server 112 (e.g., an application server) may maintain a list of medical monitors available to assist prescribers in reviewing and evaluating ophthalmic examination data and treatment data for patients being treated with a B-cell disorder therapy. A prescriber may use the prescriber-centric ocular toxicity mitigation application to submit a request for review by the medical monitor. The request may indicate (e.g., using one or more unique identifiers) the patient and the current ophthalmic examination. The server 112 may also maintain, for each medical monitor, a queue of requested reviews. The server 112 may assign a new review received from a prescriber to one of the available medical monitors and add the new review to that medical monitor's queue. Adding a new review to a medical monitor's queue may trigger an alert from the monitor-centric ocular toxicity mitigation application at the monitor-centric computing device 108. In this way, a medical monitor advantageously can be notified in real-time or near real-time during an ophthalmic examination that a prescriber has requested the medical monitor's assistance in determining an appropriate dosing modification and an appropriate dose of a component of a B-cell disorder therapy for the patient. The monitor-centric ocular toxicity mitigation application may present, at a display of the monitor-centric computing device, a user interface that includes the medical monitor's queue of reviews. The medical monitor may select one of the reviews in the queue which may cause the monitor-centric ocular toxicity mitigation application to present the ophthalmic examination data, treatment data, patient data, and the like needed to determine an appropriate dosing modification (if any) and an appropriate dose of a component of a B-cell disorder therapy to be subsequently administered to the patient. Selecting one of the reviews in the queue may trigger the monitor-centric ocular toxicity mitigation application to automatically obtain such data, e.g., from the data store(s) 110, the EMR system(s) 114, and the like. Selecting one of the reviews in the queue may also trigger the monitor-centric ocular toxicity mitigation application to automatically obtain one or more recommended dosage modifications and one or more recommended doses from the machine learning and prediction system(s) 116.

The monitor-centric ocular toxicity mitigation application may be in signal communication with the one or more EMR systems 114 to, e.g., obtain and provide medical information included in a patient's electronic medical records. The monitor-centric ocular toxicity mitigation application may be in signal communication with one or more data stores 110 to, e.g., obtain ophthalmic examination data and treatment data associated with the patient. The monitor-centric computing device 104 may be in signal communication with one or more of the servers 112 or the digital distribution platforms 118 to access or otherwise obtain the monitor-centric ocular toxicity mitigation application. The prescriber-centric ocular toxicity mitigation application may be in signal communication with the machine learning and prediction systems 116 to provide ophthalmic examination data, treatment data, and patient data and to obtain one or more recommended doses of a component of the B-cell disorder therapy that have been determined using machine learning and artificial intelligence.

The data stores 110 may be configured to store data associated with patients being treated for B-cell disorders, including cancers, using B-cell disorder therapies. The data stored at the data stores may include ophthalmic examination data, patient data, and treatment data as described herein. The data stores 110 may include one or more databases (e.g., relational databases and non-relational databases), one or more data warehouses, one or more data marts, one or more data lakes, and the like. Access to the data stores 110 may be managed by a suitable data management system (e.g., a database management system). The data store(s) 110 may be in signal communication, e.g., via the network(s) 102, with the computing devices 104, 106, and 108, the server(s) 112, the EMR system(s) 114, and the machine learning and prediction system(s) 116 to receive and provide the data stored therein.

The ophthalmic examination, treatment data, and patient data stored in the data store may provide significant benefits to medical providers and medical researchers. For example, the stored ophthalmic examination and treatment data may allow medical personnel to cross reference treatment results across the population of patients being treated with B-cell disorder therapies. A collection of ophthalmic examinations, visual acuity assessments, and corneal assessments may be compiled for further study. Data analysis techniques may be applied to the ophthalmic examination data and treatment data to map adverse corneal events to dosing decisions. Any resulting findings indicating a potential correlation or pattern between dosing decisions and adverse corneal events may be used to update the prescribing information for the component(s) of the B-cell disorder therapies. Data analysis techniques may also be applied to monitor and track patient outcomes across various demographics of the patient population which may suggest appropriate post-marketing observational studies.

The servers 112 may include different types of servers such as web servers and application servers. For example, the servers 112 may include one or more application servers that implement one or more of the patient-centric ocular toxicity mitigation application, the prescriber-centric ocular toxicity mitigation application, or the monitor-centric ocular toxicity mitigation application as a web application. The servers 112 may also include one or more web servers that manage access to the web application(s) via the network(s) 102, e.g., to provide appropriate security measures (e.g., encryption) as well as authentication and authorization of users. The patients, prescribers, and monitors, may access the web application(s) using a web browser at the patient-centric computing device 104, the prescriber-centric computing device 106, or the monitor-centric computing device. The servers 112 may also include one or more application servers and web servers to facilitate communications between the patient-centric ocular toxicity mitigation application, the prescriber-centric ocular toxicity mitigation application, or the monitor-centric ocular toxicity mitigation application as described herein.

The EMR systems 114 may store electronic medical records of the patients being treated for B-cell disorders, including cancers. The EMR data stored for the patient may include the medical histories of the patients and other patient-related data. The EMR system 114 may be in signal communication, e.g., via the network(s) 102, with the computing devices 104, 106, and 108, the data store(s) 110, the server(s) 112, and the machine learning and prediction system(s) 116 to provide the EMR data and to receive ophthalmic examination data and treatment data associated with the B-cell disorder therapies administered to the patients.

The machine learning and prediction system(s) 116 may be configured to use machine learning and artificial intelligence to determine recommended dosing modifications and recommended doses of one or more component(s) of B-cell disorder therapies used to treat B-cell disorders in patients. The machine learning and prediction system(s) 116 may include one or more predictive model(s) (e.g., machine learning models) used to predict appropriate dosing modifications and recommended doses of the component(s) of the B-cell disorder therapies. The machine learning model(s) may include one or more neural networks such as a generative adversarial network (GAN) or a consistent adversarial network (CAN) such as a cyclic generative adversarial network, a deep convolutional GAN, GAN interpolation, GAN conditional latent space, a cyclic-CAN, and the like. The neural network(s) may be trained using supervised learning, unsupervised learning, back propagation, transfer learning, stochastic gradient descent, learning rate decay, dropout, max pooling, batch normalization, long short-term memory, skip-gram, or any equivalent deep learning technique. The machine learning model(s) may be trained on data or information stored in the data store(s) 110, received from the server(s) 112 or the EMR system(s) 114, or received from the computing devices 104, 106, and 108. In this regard, the machine learning model(s) may be trained (and retrained) on the ophthalmic examination data, treatment data, patient data, and the like associated with the population of patients being treated for B-cell disorders with B-cell disorder treatment therapies. The machine learning model(s) may determine (e.g., predict), for example, whether and to what extent the current dose of a component of a B-cell disorder therapy should be adjusted for a particular patient. The machine learning model(s) may thus be configured to provide recommended dosing modifications and recommended doses to the prescribers and monitors of the B-cell disorder therapies used to treat the B-cell disorders of the patient. The machine learning model(s) may further be configured to determine and provide, for each recommended dosing modification and recommended dose, a confidence score representing the likelihood that the recommended dosing modification and the recommended dose is appropriate for the patient. The recommended dosing modification(s), recommended dose(s), and corresponding confidence score(s) may assist the prescriber or monitor in selecting an appropriate dose of a component of a B-cell disorder therapy that reduces the likelihood of ocular toxicity following a subsequent administration of the component of the B-cell disorder therapy. The machine learning model(s) may also be configured to determine one or more KVA grades and corresponding confidence scores, one or more visual acuity grades and corresponding confidence scores, and/or one or more corneal grades and corresponding confidence scores based on the ophthalmic examination data received at the machine learning and prediction system(s).

The machine learning model(s) may be trained by a set of training data to optimize parameters (e.g., the selection of the one or more data fields, weights for each data field, etc.) associated with determining the recommended dosing modification(s) and recommended dose(s). For example, the machine learning model(s) may be trained to maximize the percentage of patients having minimal ocular toxicity while maintaining a threshold level of accuracy. In this regard, machine learning and prediction system(s) 116 may be configured with or otherwise determine multiple features that are associated with ocular toxicity, B-cell disorder therapies, and patient characteristics (e.g., height, weight, age, any comorbidities, medical history, demographics, and the like). Using these features and historical ophthalmic examination data, treatment data, and patient data, the machine learning and prediction system(s) 116 may train the machine learning model(s) to determine one or more labels indicating which patients are predicted to experience ocular toxicities at various doses of a component of a B-cell disorder therapy. The machine learning and prediction system(s) 116 may receive, as input to the machine learning model(s), ophthalmic examination data, treatment data, patient data, and the like, and provide, as output, the recommended dosing modification(s), recommended dose(s), and corresponding confidence score(s). The machine learning and artificial intelligence employed by the machine learning and prediction system(s) 116 may also uncover previously unknown associations, correlations, patterns, etc. between patient characteristics, doses of the component(s) of the B-cell disorder therapies administered to those patients, and adverse corneal events that occur during the course of treatment.

The machine learning system(s) 116 may be in signal communication, e.g., via the network(s) 102, with the computing devices 104, 106, and 108, the data store(s) 110, the server(s) 112, and the EMR system(s) 114 to receive, e.g., ophthalmic examination data, treatment data, patient data, and the like used to determine the recommended dosing modification(s) and recommended dose(s) for the patient and to provide the determined recommended dosing modification(s) and recommended dose(s) to the computing devices, data store(s), server(s), and EMR system(s) for, e.g., storage and output.

The digital distribution platform(s) 118 may provide the ocular toxicity mitigation application(s) to the computing devices 104, 106, and 108. For example the digital distribution platform(s) 118 may include one or more application distribution platforms (e.g., “application store” or “app store”) that make the ocular toxicity mitigation application(s) available for download to and installation at the computing devices 104, 106, and 108. The digital distribution platform(s) 118 may include public digital distribution platform(s) and private digital distribution platform(s). The digital distribution platform(s) 118 may be in signal communication, e.g., via the network(s) 102, with the computing devices 104, 106, and 108.

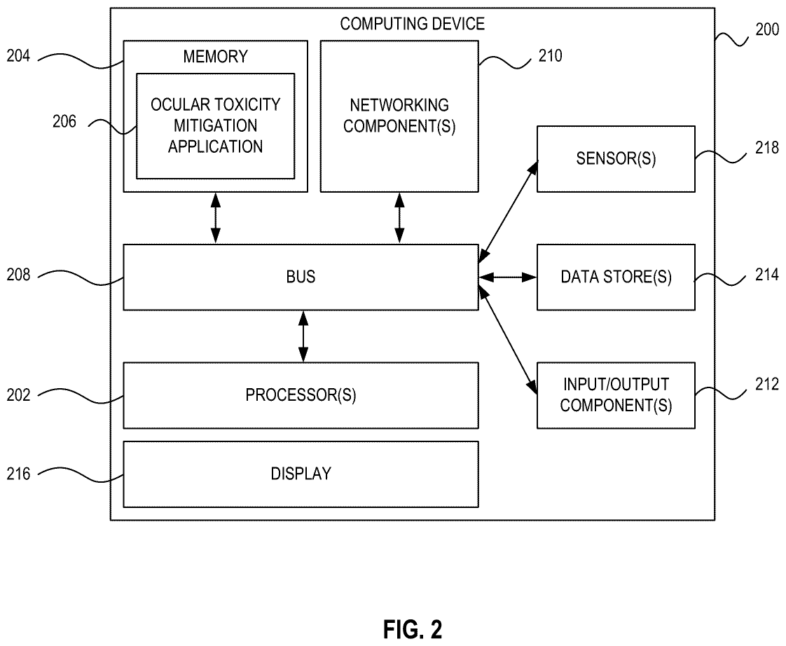

In FIG. 2, a block diagram of an example computing device 200 that may be used to implement aspects of the disclosures is shown. The computing devices illustrated in FIG. 1 and discussed above may include some or all of the components of computing device 200. The computing device 200 may be or include, for example, a laptop computer, desktop computer, tablet, e-reader, smart phone or other mobile device, portable electronic device, or other electronic device.

The computing device 200 shown by way of example in FIG. 2 includes a processor 202, a memory 204, and a bus 208. Although FIG. 2 depicts a single processor 202, the computing device 200 may include or be in communication with any number of processors 202. The processor 202 can execute one or more of the operations described herein. The processor 202 may execute instructions stored in the memory 204 to perform the operations. The processor 202 may include one processing device or multiple processing devices. Non-limiting examples of the processor 202 include a Field-Programmable Gate Array (“FPGA”), an application-specific integrated circuit (“ASIC”), a microprocessor, and the like.

The processor 202 can be communicatively coupled to the memory 204 via the bus 208. The memory 204 may include any type of memory including volatile memory such as random access memory (RAM) and non-volatile memory such as read-only memory (ROM). Non-limiting examples of the memory 204 include electrically erasable and programmable read-only memory (EEPROM), flash memory, or any other type of memory. In some examples, at least some of the memory 204 may include a non-transitory medium from which the processor 202 can read instructions. A non-transitory computer-readable medium may include electronic, optical, magnetic, or other storage devices capable of providing the processor 202 with computer-readable instructions or other program code that, when executed by the processor 202 (or multiple processors) cause the computing device 200 to perform functions as described in the present disclosures. Non-limiting examples of a non-transitory computer-readable medium include (but are not limited to) magnetic disk(s), memory chip(s), ROM, RAM, an ASIC, a configured processor, optical storage, magnetic storage, or any other non-transitory medium from which a computer processor can read instructions. The instructions can include processor-specific instructions generated by a compiler or an interpreter from code written in any suitable computer-programming language, including, for example, C, C++, C#, etc.

The memory 204 may include an ocular toxicity mitigation application 206. As described above, the ocular toxicity mitigation application 206 may be configured for use by a patient being treated for a B-cell disorder (patient-centric ocular toxicity mitigation application), by a prescriber of the B-cell disorder therapy (prescriber-centric ocular toxicity mitigation application), or by a monitor of the B-cell disorder therapy administered to the patient (monitor-centric ocular toxicity mitigation application). The ocular toxicity mitigation application 206 may include or otherwise configured to use an ocular toxicity grading algorithm configured to determine an ocular toxicity grade based at least on an ophthalmic examination of the patient as described herein. Determining the ocular toxicity grade may include determining one or more ophthalmic assessment grades for each eye of the patient and determining the ocular toxicity grade based on the one or more ophthalmic assessment grades as described herein. The ocular toxicity mitigation application 206 may also be configured to determine and output a recommended dose of a component of the B-cell disorder therapy as described herein.

The memory 204 may include one or more sets of rules for evaluating potential ocular toxicity in a patient. The sets of rules can be defined or developed by a clinical team or a healthcare provider and input into the computing device 202. For example, the one or more sets of rules may be implemented as the ocular toxicity grading algorithm 206. The computing device 202 may apply one or more rules from a rule set to data associated with the patient (e.g., ophthalmic examination data) to determine one or more corrective options (e.g., adjusting the dose of a component of the B-cell therapy). In some examples, a rule set may include one or more logical expressions.

The computing device 202 can include input/output (“I/O”) interface components 212 and additional data stores 214. The I/O interface components 212 may interface with a display 216, sensor 222, keyboard, mouse, joystick, touch-screen, additional storage 214, or any combination of these. The display 216 can include a television, computer monitor, liquid crystal display (LCD), holographic display, or any other suitable display device.

The computing device 102 may include networking component(s) 210. Networking component(s) 210 may represent one or more of any components that facilitate a network connection. In some examples, the networking component(s) 210 may facilitate both wired and wireless connections. The networking component(s) 210 may include an Ethernet interface, a modem interface, a serial interface, a USB interface, an IEEE 1394 interface, and the like. The networking component(s) 210 may also include wireless interfaces such as an IEEE 802.11 interface, a Bluetooth interfaces, or a radio interface for accessing a cellular telephone network (e.g., a transceiver/antenna for accessing CDMA, GSM, UMTS, or other mobile communications network).

The computing device 200 may include or be in signal communication with one or more sensor(s) 218. The sensor(s) 218 can be directly coupled to the computing device 200, such as with a wire, or the computing device can be in signal communication with the sensor(s) over a network (e.g., the network 104 of FIG. 1). In some examples, the sensor(s) 218 can include ophthalmic examination equipment, a blood pressure sensor, an electrocardiogram (ECG) device, a pressure sensor, a force sensor, a magneto-resistive sensor, a temperature sensor, an oxygen sensor, an X-ray device, a magnetic resonance imaging (MRI) device, a pulse sensor, a galvanic skin response sensor, an airflow sensor, a heart rate sensor, or any combination of these. The sensor(s) 128 may detect one or more characteristics of a patient and transmit sensor signals associated with the characteristics to the processor 202. In other examples, the sensor(s) 218 may include a user input device. For example, the sensor(s) 218 may include a mouse, keyboard, stylus, touch-screen display, touch pad, or any combination of these. The sensor(s) 218 may detect a user input and transmit sensor signals associated with the user input to the processor 202.

A computing device similar to the computing device 200 shown in FIG. 2 may be configured to use machine learning and artificial intelligence as described above to determine and provide one or more recommended doses of a component of a B-cell disorder therapy. That computing device may include a memory the same as or similar to memory 204, and that memory may include one or more predictive models (e.g., machine learning models) as described above as well as executable instructions (e.g., code, software applications, programs, modules, routines, and the like) to configure the predictive models (e.g., train and retrain the machine learning models).

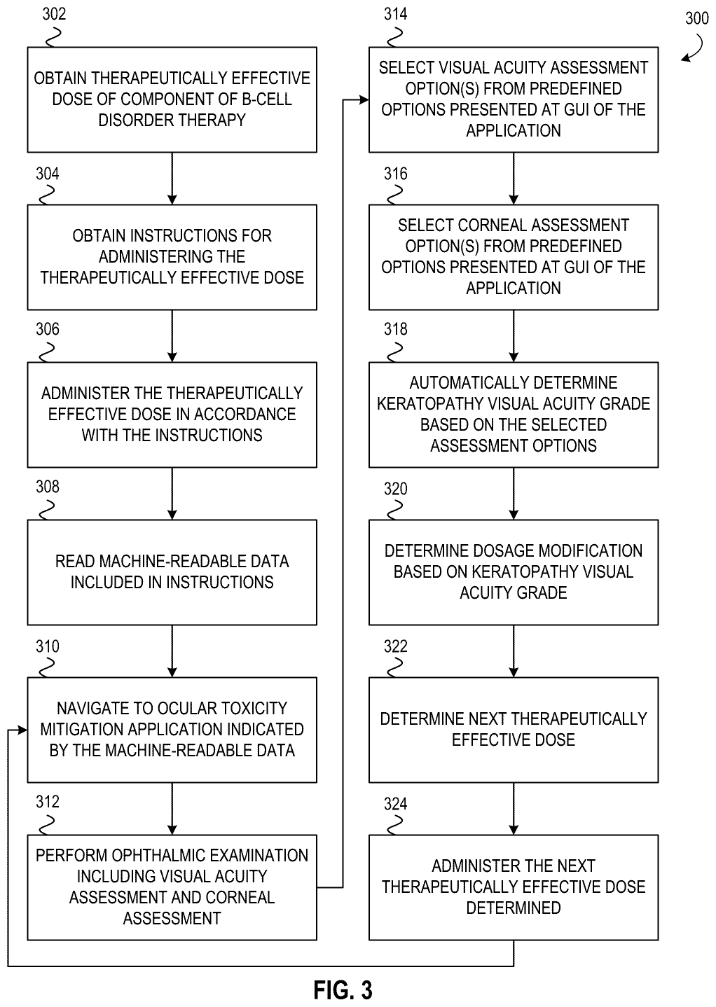

In FIG. 3, a flowchart 300 of example method steps for mitigating ocular toxicity in a patient being treated for a B-cell disorder, including cancer, is shown. In some examples, the steps shown in FIG. 3 may be performed by a single healthcare provider treating a patient for a B-cell disorder. In other examples, the steps shown in FIG. 3 may be performed by a team of healthcare providers treating the patient. For example, an ophthalmologist may perform the ophthalmic examination, an oncologist may determine a recommended dosing modification or recommended dose, and another health care provider may administer the dose recommended by the oncologist. Other examples will be appreciated with the benefit of the disclosures herein. It should be appreciated that the steps illustrated in FIG. 3 as well as the order in which they are presented are provided by way of example only and should not be interpreted as limiting in any way on the claimed subject matter. In various examples, the order of the steps may be varied, one or more steps may be omitted, and one or more additional or alternative steps may be performed. For example, not all steps illustrated in FIG. 3 may be performed when evaluating a patient to administer a subsequent dose of a B-cell disorder therapy after the patient has received an initial dose.

At step 302, a prescriber may obtain a therapeutically effective dose of a component of a B-cell disorder therapy. As an example and described herein, the component may be belantamab mafodotin (e.g., BLENREP®). As noted above, a B-cell disorder therapy may be a combination therapy. As such, a prescriber may obtain therapeutically effective doses of multiple components of a combination B-cell disorder therapy. At step 304, the prescriber may obtain instructions for administering the therapeutically effective dose of the component of the B-cell disorder therapy. The instructions for administering the component may be set forth in the prescribing information (e.g., U.S. Prescribing Information) for the component. At step 306, the prescriber may administer the therapeutically effective dose of the component in accordance with those instructions. The therapeutically effective dose administered may be an initial dose administered at the beginning of a B-cell disorder therapy or may be one of multiple doses administered to the patient over the course of the B-cell disorder therapy. As described herein, an initial ophthalmic examination of the patient may be performed prior to administering the initial dose of the component of the B-cell disorder therapy in order to obtain the patient's BCVA.

The instructions for administering the component of the B-cell disorder therapy may include machine-readable data that includes an address of a computer-executable application, e.g., an ocular toxicity mitigation application. The machine-readable data may include an optical label such as a barcode that encodes the address of the computer-executable application. The barcode may be any suitable barcode capable of encoding the address and being read by a computing device including, for example, linear barcodes (“one dimensional” barcodes) and matrix barcodes (“two dimensional” barcode), and their equivalents. One example of a suitable matrix barcodes that may be employed is a quick response (QR) code. The machine-readable data may additionally or alternatively include a link (e.g., a hyperlink) that includes the address to the computer-executable application. The machine-readable data may include respective addresses to each of the patient-centric ocular toxicity mitigation application, the prescriber-centric ocular toxicity mitigation application, and the monitor centric ocular toxicity mitigation application. In this regard, as an example, the machine-readable data may include a patient QR code, a prescriber QR code, and a monitor QR code in order to allow each of the patient, prescriber, and monitor to navigate to the appropriate ocular toxicity mitigation application. Additional and alternative types of optical labels may be employed

At step 308, the prescriber may read the machine-readable data included in the instructions in order to obtain the address for the computer-executable application (e.g., the prescriber-centric ocular toxicity mitigation application). Reading the machine-readable data may include optically scanning the machine-readable data (e.g., a QR code) using an optical scanner (e.g., a camera) of a computing device (e.g., the prescriber-centric computing device) and decoding the address encoded therein. Reading the machine-readable data may also include processing the link that includes the address to the computer-executable application.

At step 310, the prescriber may use the prescriber-centric computing device to navigate to the computer-executable application indicated by the machine-readable data, e.g., the prescriber-centric ocular toxicity mitigation application. Navigating to the ocular toxicity mitigation application may include determining whether the ocular toxicity mitigation application is installed at the computing device; if so, automatically launching the ocular toxicity mitigation application; and if not, automatically accessing a digital distribution platform (e.g., an “app store”) that makes the ocular toxicity mitigation application available for download and installation at the prescriber-centric computing device. Navigating to the ocular toxicity mitigation application may also include using a web browser to access a web-based ocular toxicity mitigation application located at the address encoded in the machine readable data.

Steps similar to steps 304, 308, and 310 may be employed by the patient-centric computing device and the monitor-centric computing device to respectively navigate to the patient-centric ocular toxicity mitigation application and the monitor-centric ocular toxicity mitigation application.

At step 312, the prescriber may perform an ophthalmic examination, including a visual acuity assessment and a corneal assessment, using the prescriber-centric ocular toxicity mitigation application as described herein. The prescriber-centric ocular toxicity mitigation application may provide a graphical user interface (GUI) that presents, on one or more screens of the GUI, predefined and selectable options for indicating a visual assessment and a corneal assessment of a patient during the ophthalmic examination. By using predefined and selectable assessment options, the prescriber-centric ocular toxicity mitigation application reduces the chance of erroneous visual acuity assessments and corneal assessments thus improving the accuracy of the KVA grade determined for the patient and, in turn, improving dosing decisions for the patient that can lead to better patient outcomes with respect to the underlying B-cell disorder and the subsequent ocular toxicity for the remainder of the treatment. As such, at step 314, the prescriber may select one or more visual assessment options from the predefined, selectable visual acuity options presented at the GUI of the prescriber-centric ocular toxicity mitigation application. Similarly, at step 316 the prescriber may select one or more corneal assessment options from the predefined, selectable corneal assessment options presented at the GUI of the prescriber-centric ocular toxicity mitigation application.

At step 318, the prescriber-centric ocular toxicity mitigation application may automatically determine a KVA grade based on the visual acuity assessment options and the corneal assessment options selected by the prescriber. Determining the KVA grade may include automatically determining, for each eye of the patient, a visual acuity grade and a corneal grade. The visual acuity grades may be based on the visual acuity assessment options selected by the prescriber. As described above, the visual acuity grades may indicate or otherwise characterize any changes to the patient's baseline BCVA which may have declined or remained the same since a prior visual acuity assessment. The corneal grades may indicate the corneal state of the patient including the presence of any keratopathies. The KVA grade may be based on the visual acuity grades and corneal grades. The KVA grade may be or otherwise include a numerical score (e.g., 1-10, 1-100, etc.), letter grade (e.g., A-Z), and the like. For example, the KVA grade may be based on a sum of the visual acuity grades and corneal grades or an average or a weighted average of the visual acuity grades and corneal grades. The prescriber-centric ocular toxicity mitigation application may be configured to automatically determine a visual acuity grade based on receiving selection of all necessary visual acuity assessment options (e.g., baseline BCVA and current visual acuity). The prescriber-centric ocular toxicity mitigation application may also be configured to automatically determine a corneal grade based on receiving selection of all necessary corneal assessment options. The prescriber-centric ocular toxicity mitigation application may be configured to automatically determine the KVA grade based on receiving selection of all necessary visual acuity assessment options and corneal assessment options (e.g., for each of the patient's left eye and right eye). Put differently, selecting sufficient visual acuity assessment options and corneal assessment options may trigger the prescriber-centric ocular toxicity mitigation application to automatically determine one or more of the visual acuity grade(s), the corneal grade(s), and the overall KVA grade for the patient.

At step 320, a dosage modification for a component of a B-cell disorder being used to treat the patient may be determined based on the overall KVA grade as described herein. The dosage modification may include changing the dose (e.g., increasing or decreasing the dose), keeping the dose the same, or discontinuing treatment (e.g., until the patient recovers from any adverse corneal events). As described above, the prescriber-centric ocular toxicity mitigation application may be configured to determine and output one or more recommended dosage modifications. The determination of the dosage modification likewise may be automatic and triggered by a determination of the overall KVA grade after sufficient visual acuity assessment options and corneal assessment options have been selected. The determination of the dosage modification also may be triggered by receiving user input from the prescriber requesting a recommended dosage modification after the overall KVA grade has been determined.

As described above, an ocular toxicity mitigation application may be configured to provide recommended doses and/or dosage modifications to a component of a B-cell disorder therapy based on the determined KVA grade as shown in the following examples:
a B-cell disorder therapy (for example belantamab mafodotin) is administered at a first dose (for example 2.5 mg/kg), prior to next administration a decline from baseline by 1 line on Snellen Visual Acuity (or other equivalent decline using an alternative visual acuity unit) is detected in the subject, treatment is continued at same dose (for example 2.5 mg/kg)
a B-cell disorder therapy (for example belantamab mafodotin) is administered at a first dose (for example 2.5 mg/kg), prior to next administration a decline from baseline by 2 or 3 lines on Snellen Visual Acuity (or other equivalent decline using an alternative visual acuity unit) and not worse than 20/200 is detected in the subject, withhold B-cell disorder therapy (for example belantamab mafodotin) until improvement in both corneal examination findings and change in BCVA to Grade 1 or better and resume at same dose
a B-cell disorder therapy (for example belantamab mafodotin) is administered at a first dose (for example 2.5 mg/kg), prior to next administration a decline from baseline by more than 3 lines on Snellen Visual Acuity (or other equivalent decline using an alternative visual acuity unit) and not worse than 20/200 is detected in the subject, withhold B-cell disorder therapy (for example belantamab mafodotin) until improvement in both corneal examination findings and change in BCVA to Grade 1 or better and resume at reduced dose (for example 1.9 mg/kg)
a B-cell disorder therapy (for example belantamab mafodotin) is administered at a first dose (for example 2.5 mg/kg), prior to next administration a Snellen Visual Acuity (or other equivalent decline using an alternative visual acuity unit) worse than 20/200 is detected in the subject, consider permanent discontinuation of B-cell disorder therapy (for example belantamab mafodotin). If continuing treatment, withhold B-cell disorder therapy (for example belantamab mafodotin) until improvement in both corneal examination findings and change in BCVA to Grade 1 or better and resume at reduced dose (for example 1.9 mg/kg)

As also described above, recommended dosage modifications may be received from a medical monitor via a monitor-centric ocular toxicity mitigation application as well as a machine learning and prediction system. A request for a recommended dosage modification may be sent automatically to the monitor-centric ocular toxicity mitigation application or a machine learning and prediction system. The request(s) likewise may be triggered by a determination of the overall KVA grade after sufficient visual acuity assessment options and corneal assessment options have been selected.

At step 322, the next therapeutically effective dose of the component of the B-cell disorder may be determined. As also described above, one or more recommended doses may be received from the medical monitor via the monitor-centric ocular toxicity mitigation application as well as a machine learning and prediction system. The request for a recommended dosage modification may include a request for a recommended dose. Additionally or alternatively, a separate request for a recommended dose may be sent. The request for a recommended dose may be sent automatically to the monitor-centric ocular toxicity mitigation application or a machine learning and prediction system. The request(s) likewise may be triggered by a determination of the overall KVA grade after sufficient visual acuity assessment options and corneal assessment options have been selected. The request for the recommended dose also may be triggered by receiving user input from the prescriber requesting a recommended dose of the component of the B-cell disorder therapy.

At step 324, the prescriber may administer a subsequent therapeutically effective dose of the component of the B-cell disorder therapy. As described herein, the subsequent therapeutically effective dose may be determined based on one or more of the input of the prescriber, a monitor of the B-cell disorder therapy, or a machine learning and prediction system. After the subsequent therapeutically effective dose is administered to the patient, steps 310-324 may be repeated in order to continue to monitor the patient for ocular toxicity during the B-cell disorder therapy and make further adjustments to a dose of a component of the B-cell disorder therapy while treatment continues.

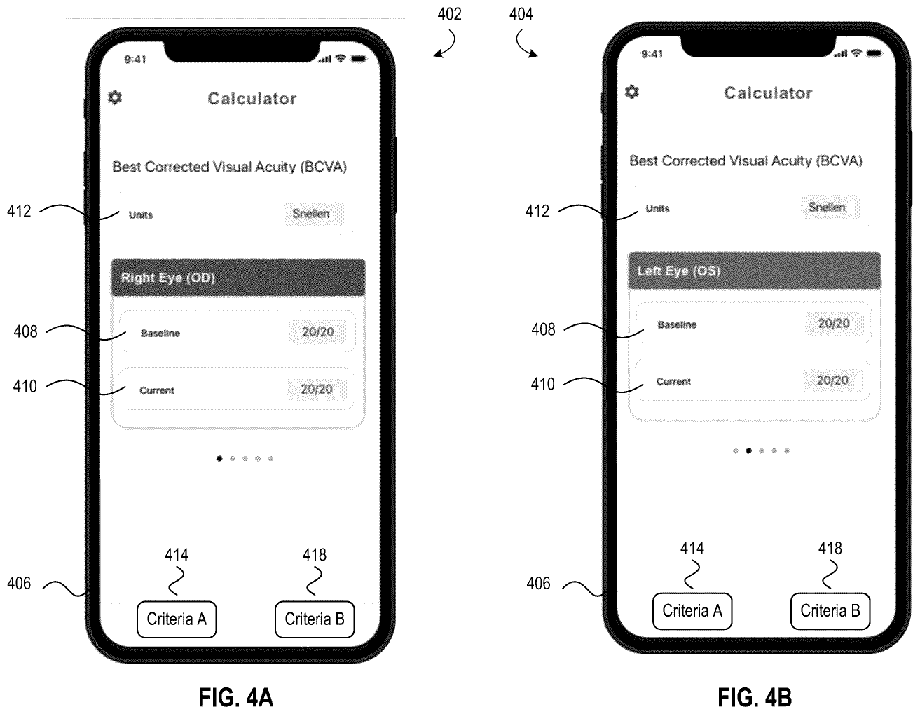

In FIG. 4A and FIG. 4B, example screens 402 and 404 of a graphical user interface (GUI) for receiving user input indicating selection of visual acuity assessment options at a prescriber-centric ocular toxicity mitigation application are shown. The example screens 402 and 404 are provided by a prescriber-centric ocular toxicity mitigation application installed at a mobile computing device 406 and are presented at a display of the mobile computing device 406. The example screens 402 and 404 are configured to receive, e.g., during an ophthalmic examination of a patient, input from the prescriber indicating a patient's baseline and current BCVA. The example screens 402 and 404 include predefined and selectable visual acuity assessment options 408 and 410 from which the prescriber may indicate the patient's baseline visual acuity (408) and current visual acuity (410). The visual acuity assessment data of the ophthalmic examination data obtained during the ophthalmic examination may thus include, represent, or otherwise correspond to the visual acuity assessment options selected by the prescriber. As described above, the prescriber-centric ocular toxicity mitigation application may be configured to automatically populate a patient's baseline BCVA based on patient data received from a previous ophthalmic examination stored, e.g., a local or remote data store, an EMR record for the patient stored at an EMR system, and the like. The example screens 402 and 404 are also configured to receive selections of visual acuity assessment options for each eye of the patient, e.g., the right eye (screen 402) and the left eye (screen 404).

The GUIs 402 and 404, in this example, also include predefined and selectable visual acuity unit options 412 indicating the visual acuity unit of the visual acuity assessment options. For example, the predefined and selectable visual acuity unit options 412 may allow the prescriber to select from various visual acuity units such as Snellen (feet), Snellen (metric), MAR, logMar, decimal, and the like. The predefined and selectable visual acuity assessment options 408 and 410 may depend on the visual acuity unit option selected. For example, the prescriber-centric ocular toxicity mitigation application may be configured to automatically populate the visual acuity assessment options based on the visual acuity unit option that the prescriber selects. Selecting a different visual acuity unit option may cause the prescriber-centric ocular toxicity mitigation application to automatically update the visual acuity assessment options 408 and 410 that are available for selection.

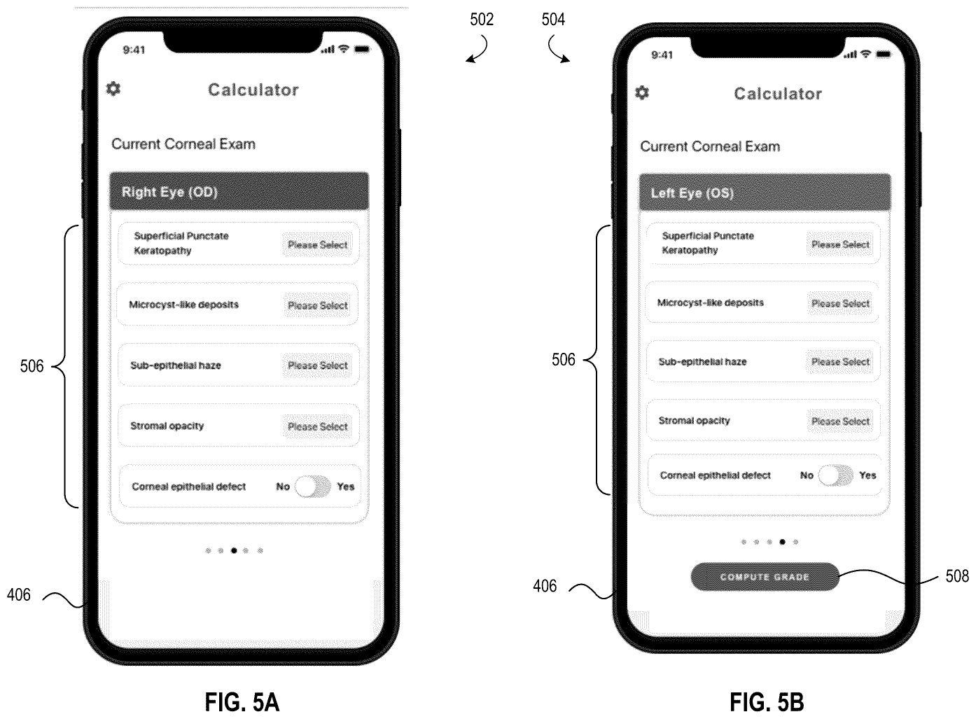

In FIG. 5A and FIG. 5B, example screens 502 and 504 of a GUI for receiving user input indicating selection of corneal assessment options at prescriber-centric ocular toxicity mitigation application are shown. As seen in FIGS. 5A-5B, the screen 502 and 504 are configured to receive user input indicating the corneal state of both the patient's right eye (screen 502) and left eye (screen 504). The predefined and selectable corneal assessment options 506 in this example include whether the patient has any superficial punctuate keratopathy, any microcyst-like deposits, any sub-epithelial haze, any stromal opacity, or a corneal ulcer or corneal erosion. As such, the predefined and selectable corneal assessment options 506 include predefined and selectable superficial keratopathy assessment options and multiple sets of predefined and selectable corneal event options. The sets of predefined and selectable corneal event options include options for microcyst-like deposits, sub-epithelial haze, stromal opacity, and any corneal epithelial defects. One of the screens (e.g., screen 504) may also include a user input element 508 (e.g., a button) to trigger calculation of one or more of the visual acuity grade(s), corneal grade(s), or the overall KVA grade. As described herein, such calculation(s) may be automatic and triggered by, e.g., upon selection of sufficient visual acuity assessment options (e.g., a threshold quantity of visual acuity assessment options) and selection of sufficient corneal assessment options (e.g., a threshold quantity of corneal assessment options).

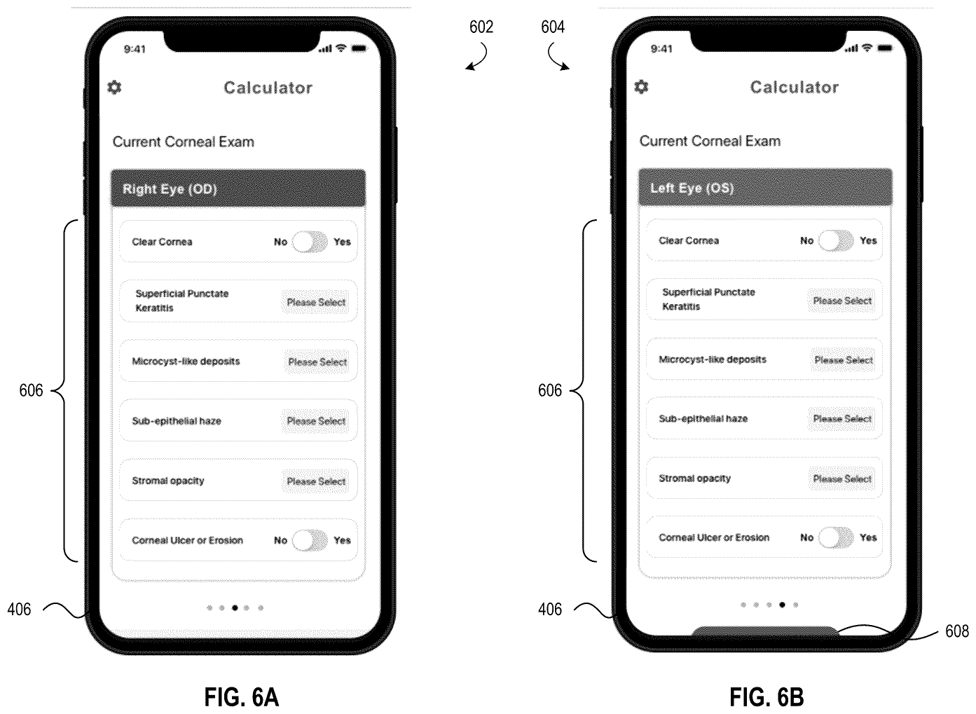

In FIGS. 6A and 6B, examples of alternative screens 602 and 604 of a GUI for receiving user input indicating selection corneal assessment options at a prescriber-centric ocular toxicity mitigation application are shown. The screens 602 and 604 may include different predefined and selectable corneal assessment options as compared to those in screens 502 and 504 discussed with reference to FIG. 5A and FIG. 5B above. Medical providers may employ different criteria for assessing the corneal state of a patient. In some examples, a prescriber-centric ocular toxicity mitigation application may be configured to toggle between the different criteria employed and respectively present predefined and selectable corneal assessment options for each set of criteria that may be employed. Although only two sets of criteria are respectively shown in FIGS. 5A-5B and 6A-6B, it will be appreciated that a prescriber-centric ocular toxicity mitigation application may be configured to toggle between any number of sets of criteria for assessing the corneal state of the patient.

Returning briefly to FIG. 4A and FIG. 4B, the prescriber-centric ocular toxicity mitigation application may include predefined and selectable corneal assessment criteria options 414 and 418 to toggle between the respective sets of corneal assessment criteria shown by way of example in FIGS. 5A-B and FIGS. 6A-B. The predefined and selectable corneal assessment options presented at the prescriber-centric ocular toxicity mitigation application may depend on which corneal assessment criteria option is selected. For example, selecting corneal assessment criteria option 414 may cause the prescriber-centric ocular toxicity mitigation application to present the corneal assessment options shown in FIGS. 5A-5B, and selecting corneal assessment criteria option 418 may cause the prescriber-centric ocular toxicity mitigation application to present the corneal assessment options shown in FIGS. 6A-6B.

As seen in FIGS. 6A-6B, the screens 602 and 604 are configured to receive user input indicating the corneal state of both the patient's right eye (screen 602) and left eye (screen 604). The predefined and selectable corneal assessment options 606 in this alternative example include whether the patient has a clear cornea, any superficial punctuate keratitis, any microcyst-like deposits, any sub-epithelial haze, any stromal opacity, or a corneal ulcer or corneal erosion. Here again, the predefined and selectable corneal assessment options 606 include predefined and selectable superficial keratopathy assessment options and multiple sets of predefined and selectable corneal event options. The sets of predefined and selectable corneal event options include options for a clear cornea, microcyst-like deposits, sub-epithelial haze, stromal opacity, and any corneal ulcer or erosion. One of the screens (e.g., screen 604) may also include a user input element 608 (e.g., a button) to trigger calculation of one or more of the visual acuity grade(s), corneal grade(s), or the overall KVA grade and trigger presentation of these grades at the GUI. As described herein, the calculation and presentation of the grade(s) may be automatic and caused by, e.g., selection of sufficient visual acuity assessment options and selection of sufficient corneal assessment options.

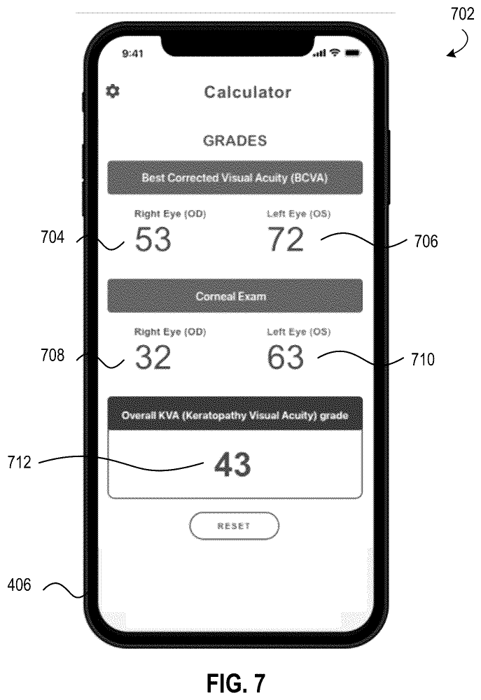

In FIG. 7, an example 702 of a GUI for presenting a keratopathy visual acuity grade at a prescriber-centric ocular toxicity mitigation application is shown. As seen in FIG. 7, the example screen 702 includes respective visual acuity grades 704 and 706 for both the patient's right eye (grade 704) and left eye (grade 706). The example screen 702 also includes respective corneal grades 708 and 710 for both the patient's right eye (grade 708) and left eye (grade 710). The example screen 702 may also include an overall KVA grade 712 for the patient. As described herein, the overall KVA grade may be based on the visual acuity grades 704 and 706 and based on the corneal grades 708 and 710. The numbers for these grades shown in FIG. 7 are provided simply as placeholders and do not necessarily represent the grades a prescriber-centric ocular toxicity mitigation application may be configured to calculate in any particular implementation.

The display of the computing device 406 may include a touchscreen for receiving the user input. Additional and alternative types of input devices may be employed to provide the user input at a computing device (e.g., a stylus, a mouse, a keyboard, and the like). The predefined and selectable options may be presented in various forms including drop-down menus, pie menus, checkboxes, combo boxes, list boxes, toggle switch, radio buttons, sliders, spinners, and the like. In some examples, the predefined and selectable options may not be editable by the prescriber. In some examples, the GUI may include no editable user input elements. In some examples, the GUI may include one or more editable user input elements such as a text entry field for receiving, e.g., prescriber notes during the ophthalmic examination.

The GUI of the prescriber-centric ocular toxicity mitigation application may present the visual acuity assessment options, the corneal assessment options, and the overall KVA grade on different screens, e.g., as shown in FIGS. 4A-4B, 5A-5B, 6A-6B, and 7. The prescriber may navigate between screens using touch gestures (e.g., by swiping left or right). The prescriber-centric ocular toxicity mitigation application may be configured to navigate between screens of the GUI based on other types of user input (e.g., input using a stylus, a mouse, a keyboard, etc.). In other examples, the prescriber-centric ocular toxicity mitigation application may be configured to present the visual acuity assessment options, the corneal assessment options, and the overall KVA grade on a single screen of the GUI.

The prescriber-centric ocular toxicity mitigation application may also be configured to display ophthalmic examination data for previous ophthalmic examinations of the patient. In this regard, the prescriber-centric ocular toxicity mitigation application may be configured to present a selectable list of patients being treated by the prescriber and, upon selection of a particular patient from that list, display a list of previous ophthalmic examinations of the selected patient (e.g., in addition to other patient data for the selected patient such as the patient's medical history, prescriber notes, and the like). The prescriber may then select one of the previous ophthalmic examinations, and the screens of the GUI (e.g., the screens discussed above with reference to FIG. 4A-4B, 5A-5B, 6A-6B, or 7) may be automatically populated with the appropriate visual acuity assessment options and corneal assessment options selected by the prescriber (or another medical provider) during the previous ophthalmic examination.

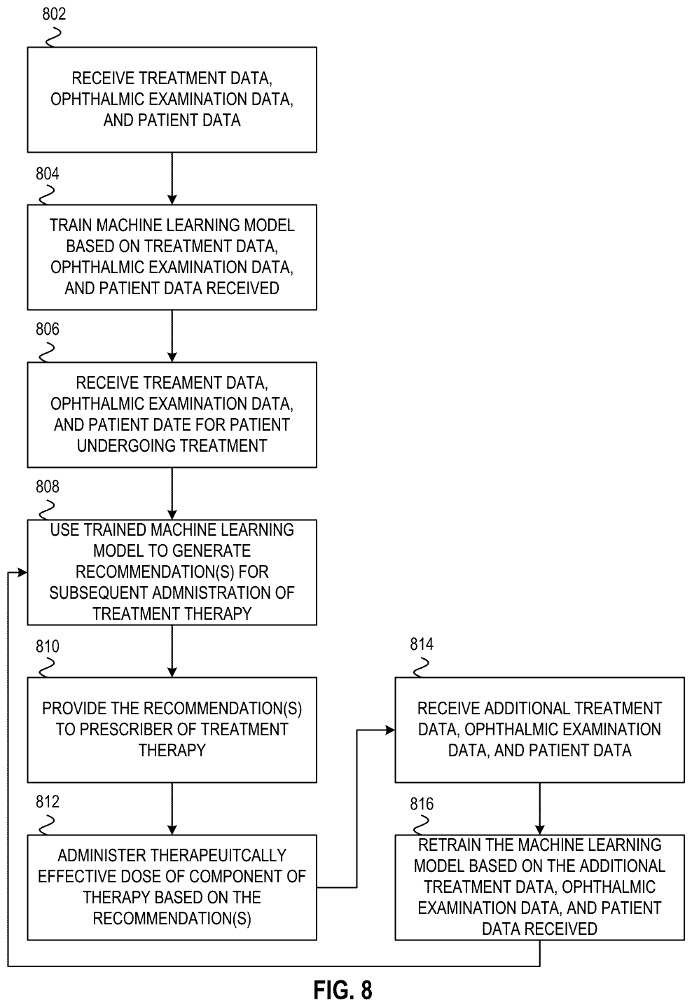

In FIG. 8, a flowchart 800 of example method steps for training a machine learning model for mitigating ocular toxicity and obtaining a dosing recommendation from the trained machine learning model is shown. Other examples will be appreciated with the benefit of the disclosures herein. It should be appreciated that the steps illustrated in FIG. 8 as well as the order in which they are presented are provided by way of example only and should not be interpreted as limiting in any way on the claimed subject matter. In various examples, the order of the steps may be varied, one or more steps may be omitted, and one or more additional or alternative steps may be performed. For example, not all steps illustrated in FIG. 8 may be performed when using a machine learning model to provide recommendations associated with a subsequent dose of a B-cell disorder therapy after a patient has received an initial dose.

At step 802, a machine learning and prediction system may receive treatment data, ophthalmic examination data, and patient data. This data may represent a corpus of therapy treatments and corresponding patient outcomes in terms of the underlying disorder being treated as well as the occurrence of any side effects. The corpus, therefore, may include data for administrations of therapies associated with patient recovery or remission, associated with adverse corneal events or no adverse corneal events, and the like. At least some of the data in the corpus may be labeled to indicate administrations that have been adjudicated to be associated with appropriate dosing decisions as well as administrations where dosage adjustments may have been the more appropriate dosing decision. The corpus may include data associated with actual therapies administered to actual patients. The corpus may also include synthetic data randomly or manually generated. For training purposes, the synthetic data may include data associated with dosages known to be improper for administration to patients in certain contexts, e.g., maintaining a certain dose despite the presence of significant keratopathies where the appropriate dosing decision would be to cease treatment until the patient recovers from the ocular toxicity side effects. Such labels facilitate the training of the machine learning model. The machine learning and prediction system may store the received data in a local data store or a remote data store (e.g., data store 110 in FIG. 1). In this regard, construction of the initial machine learning models and their corresponding priors may be based on historical treatment data, ophthalmic examination data, and patient data. As described below, as new treatment data, ophthalmic examination data, and patient data is obtained, the priors may be updated based on that data, and the machine learning models may be retrained (or rebuilt entirely) to improve performance and accuracy. A machine learning and prediction system may also be configured to obtain and store performance metrics for the machine learning models (e.g., for different machine models or different versions/iterations of a particular machine model) in order to, e.g., identify outliers and evaluate the ability of the models to provide accurate and consistent predictions.

At step 804, the machine learning model may be trained on a portion of the received treatment data, ophthalmic examination data, and patient data. In this regard, the corpus may be divided into a set of training data and a set of test data. The machine learning model may be or otherwise include, for example, one or more neural networks, clustering models, decision trees, random forests, and the like. Any suitable training techniques may be employed, e.g., supervised or unsupervised learning, regression, or classification. The machine learning model may be iteratively trained until a threshold level of confidence is achieved with the test data of the corpus. With the machine learning model sufficiently trained, the machine learning and prediction system may provide dosing recommendations as described herein. In some examples, an ensemble of multiple machine learning models may be trained to facilitate dosing recommendations and monitoring of adverse ocular (e.g., corneal) events. The inputs to one or more machine learning models of this ensemble may include, for example, patient data such as respective histories of patients' visual acuity and respective histories of patients' corneal examination findings, respective histories of changes to patients' visual acuity and/or corneal examination findings paired with corresponding dosing information indicating one or more doses administered to the patients, historical EHR/EMR data associated with the patients, genetic data and/or biomarker data associated with the patients (e.g., identifying patient sub-populations that may be associated with an increased generic risk of adverse ocular events), whole genome sequencing (WGS) data and/or next generation sequence data (NGS) associated with the patients, omics data associated with the patients (e.g., genomics data, proteomics data, transcriptomics data, pharmacogenomics data, phenomics data, metabolomics data, lipid analysis, protein analysis, etc.), demographic data associated with the patients (e.g., age, gender, body mass index (BMI), etc.), non-health related data associated with the patients (e.g., patient survey data regarding work status and work history, family status and family history, exposure to environmental factors such as contaminants, pollutants, climate, living conditions, etc.), and eye imaging data.

At step 806, the machine learning and prediction system having a sufficiently trained machine learning model may receive treatment data, ophthalmic examination data, and patient data for a patient undergoing treatment, e.g., for a B-cell disorder. At step 808, the machine learning and prediction system may use the trained machine learning model to generate one or more recommendations associated with a subsequent administration of a therapy being used to treat the patient, e.g., a B-cell disorder therapy. The recommendation(s) may include one or more of a recommended dosage modification and a recommended dose. A recommendation may include a confidence score (e.g., from 0% up to and including 100%) determined by the machine learning and prediction system. At step 810, the machine learning and prediction system may provide the recommendation(s) to, e.g., the prescriber of the treatment therapy as described herein. Providing the recommendation(s) to the prescriber of the treatment therapy may include providing the recommendation(s) to medical providers associated with the prescriber that administer the therapy on behalf of or at the direction of the prescriber. At step 812, the prescriber may administer a therapeutically effective dose of a component of the therapy based on the recommendation(s) received. As noted above an ensemble of models may be trained on various types of patient data. As such, this ensemble of machine learning models may include a baseline model configured to provide dosing recommendations based only on screening and patient demographics (e.g., age, gender, BMI, etc.), an ongoing dosage recommendation model configured to provide only dosage modification recommendations (e.g., increase dose, decrease dose, suspend dosing, discontinue treatment altogether), an adverse event prediction and intervention model configured to indicate whether a patient is trending toward a potential adverse event (e.g., an adverse ocular event such as an adverse corneal event), a post-adverse event evaluation model configured to provide recommendations on whether and/or when to continue or discontinue dosing based on updated KVA grades. The ocular toxicity mitigation applications described above may be configured to obtain (e.g., request) the output from one or more of the machine learning models.

At step 814, the machine learning and prediction system may receive additional treatment data, ophthalmic data, and patient data in order to retrain the machine learning model in order to improve its accuracy. At step 816, therefore, the machine learning and prediction system may retrain its machine learning model based on the additional data received. Steps 808-816 may thus be repeated to iteratively retrain the machine learning model in order to improve the accuracy of the machine learning model and the recommendation(s) generated on behalf of patients undergoing treatment.

An example scenario of using machine learning models to mitigate ocular toxicity may be as follows. Initial treatment data, ophthalmic examination data, and patient data may be used to train and test one or more machine learning models as described above. The machine learning model(s) may be used to inform a initial (e.g., baseline) dosing recommendation. During the dosing regimen (e.g., dosing cycle), ophthalmic examination data is obtained and stored. One or more appropriate machine learning model may be selected, e.g., based on the data that is available. Different machine leaning models may be selected depending on whether certain types of data are available. As one example, the machine learning model(s) selected may be different depending on whether EMR/EHR data is available, depending on which types of patient data are available (e.g., patient survey data), and the like. One or more machine learning models may then be used to determine various aspects of the dosing regimen. For example, one machine learning model may be used to determine a recommended dose, another machine learning model may be used to predict the likelihood of a future adverse event, and a further machine learning model may be used to determine whether any dosing suspension thresholds have been satisfied (e.g., the occurrence of one or more adverse events). The dosing decision (e.g., increase, decrease, suspend, discontinue) may be stored in a data store to improve the machine learning model(s). For example, an appropriate dose may be determined and administered to the patient, and the dosing information may be stored for subsequent reconstruction and/or retraining of one or more of the existing data models and/or construction of new data models. The machine learning model(s) may be used at the time of care to provide health care providers (e.g., prescribers, monitors, dose administrators) with real-time feedback (e.g., at the time of care) to facilitate real-time dosing decisions. Health care providers (e.g., monitors of the dosing regimens) may also provide input to update, refine, and retrain the machine learning models.

The disclosures provided herein may be employed to mitigate ocular toxicity arising from additional and alternative therapies including, for example, those listed above in Table 1. In this regard, a prescriber-centric ocular toxicity mitigation application may be configured to grade the ocular state of a patient being treated with additional or alternative therapies. In some examples, a prescriber-centric ocular toxicity mitigation application may be configured to provide a GUI having predefined and selectable ocular toxicity options corresponding to any of the ocular toxicity issues listed in Table 1 above for the respective therapies. In some examples, a prescriber-centric ocular toxicity mitigation application may be configured to provide a GUI with a selectable list of different therapies with associated ocular toxicity issues and configure the predefined and selectable ocular toxicity options based on the therapy the prescriber selects. A prescriber-centric ocular toxicity mitigation application may be configured to receive updates to the various therapies it is configured to grade. The update may include indication of a new therapy and corresponding ocular toxicity criteria that is presented as one or more sets of predefined and selectable ocular toxicity options. The update may also include new ocular toxicity criteria for an existing therapy, and the prescriber-centric ocular toxicity mitigation application may update the associated screens for the existing therapy to include an option to toggle between the different sets of ocular toxicity criteria as described above. The disclosures provided herein may also be employed to grade the state of a patient being treated with additional and alternative therapies associated with known side effects other than ocular toxicity issues. The disclosures herein directed to training a machine learning model may also be employed to train one or more machine learning models that respectively provide recommendations of dosage modifications and corresponding dosages for the therapies included in Table 1 above.

In some examples, an ophthalmic examination may include imaging the cornea, e.g., using optical coherence tomography, in order to obtain an image of the cornea. That image may be associated with the KVA grade determined for the patient. A library of corneal images and associated KVA grades thus may be compiled over time. A machine learning model may be trained on the corneal images and configured to provide a corneal grade (and, e.g., a corresponding confidence score) for a patient based on a corneal image of the patient's eye that is received as input at the trained machine learning model. A computing device may be configured to use that trained machine learning model to automatically determine the corneal grade(s) for the patient based on the corneal image(s) obtained during an ophthalmic examination of the patient. In some examples, the corneal grade(s) for the patient may be based on the corneal image(s) alone or based on a combination of the corneal image(s) and the corneal assessment(s) obtained for the patient.

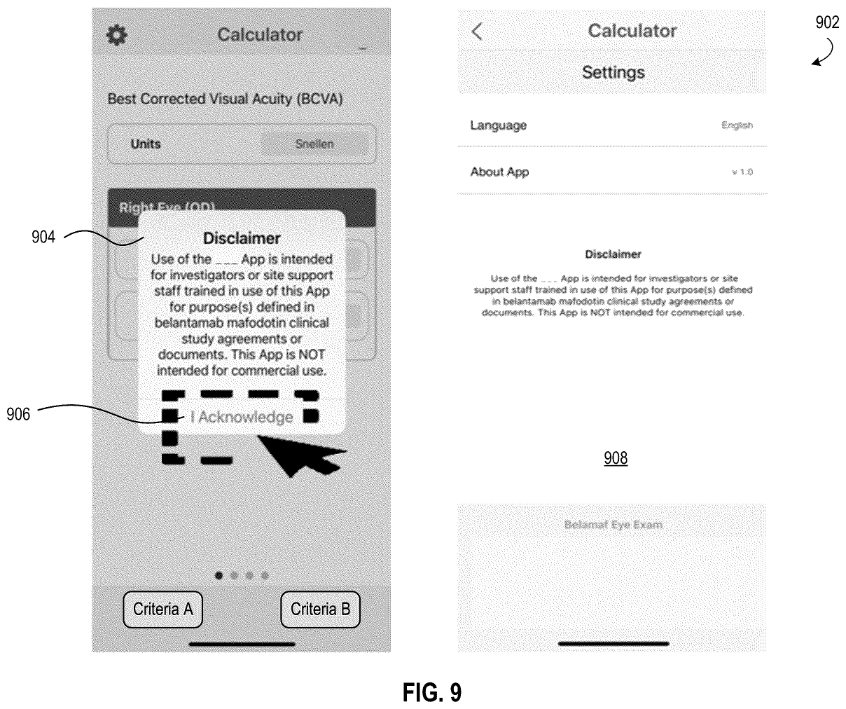

In FIG. 9, an example screen 902 of a GUI of another example of an application for mitigating ocular toxicity mitigation application is shown which, like above, may be referred to as an ocular toxicity mitigation application. The example screen 902 may be provided by a prescriber-centric ocular toxicity mitigation application installed at a mobile computing device and are presented at a display of the mobile computing device. When a user first downloads and installs the ocular toxicity mitigation application on the mobile device, a pop-up 904 may be displayed that includes a disclaimer and require the user to select a user interface element 904 acknowledging the disclaimer before using the ocular toxicity mitigation application. The pop-up 904 may be a one-time pop-up and displayed to the user when the user launches the ocular toxicity mitigation application for the first time after download. Thereafter, the user may display the disclaimer by navigating to a settings screen 908 while using the ocular toxicity mitigation application. In some instances, use of the ocular toxicity mitigation application may be restricted to those trained in using the ocular toxicity mitigation application such as site support staff and/or investigators monitoring for ocular toxicity in patients being treated for a B-cell disorder using one or more B-cell disorder therapies known to be associated with ocular toxicity side effects.

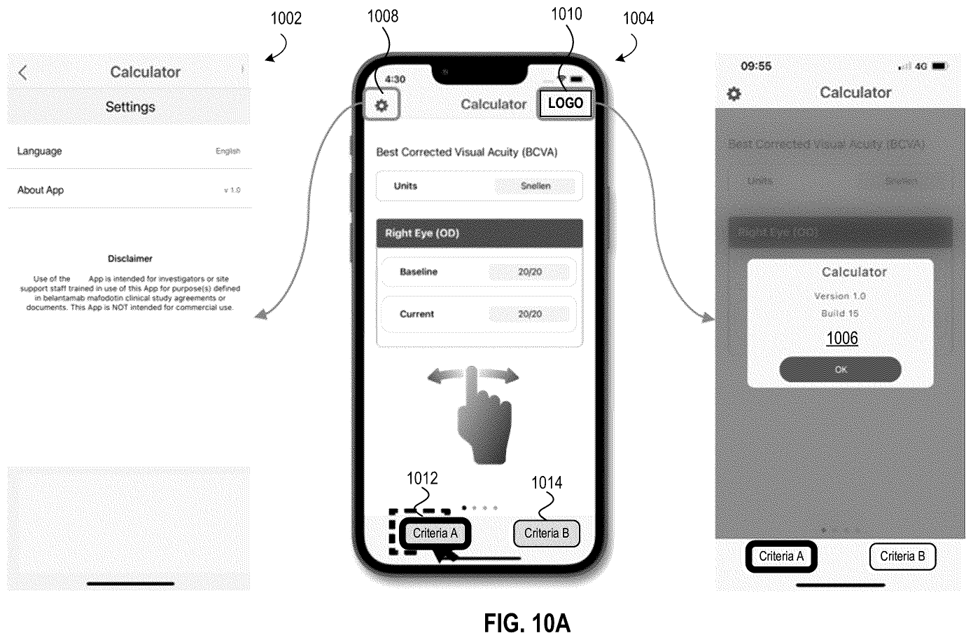

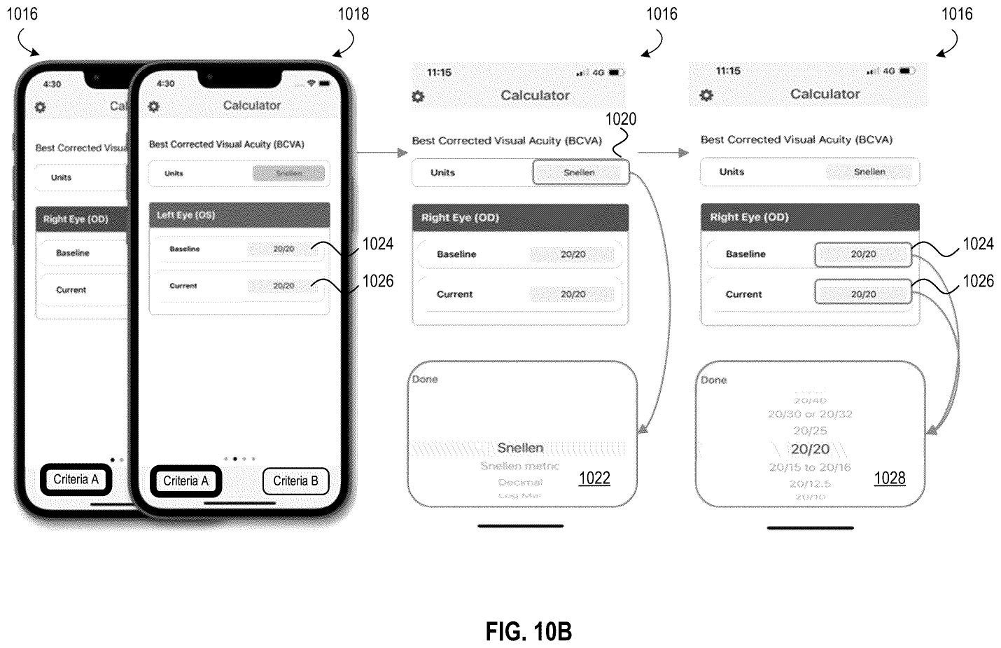

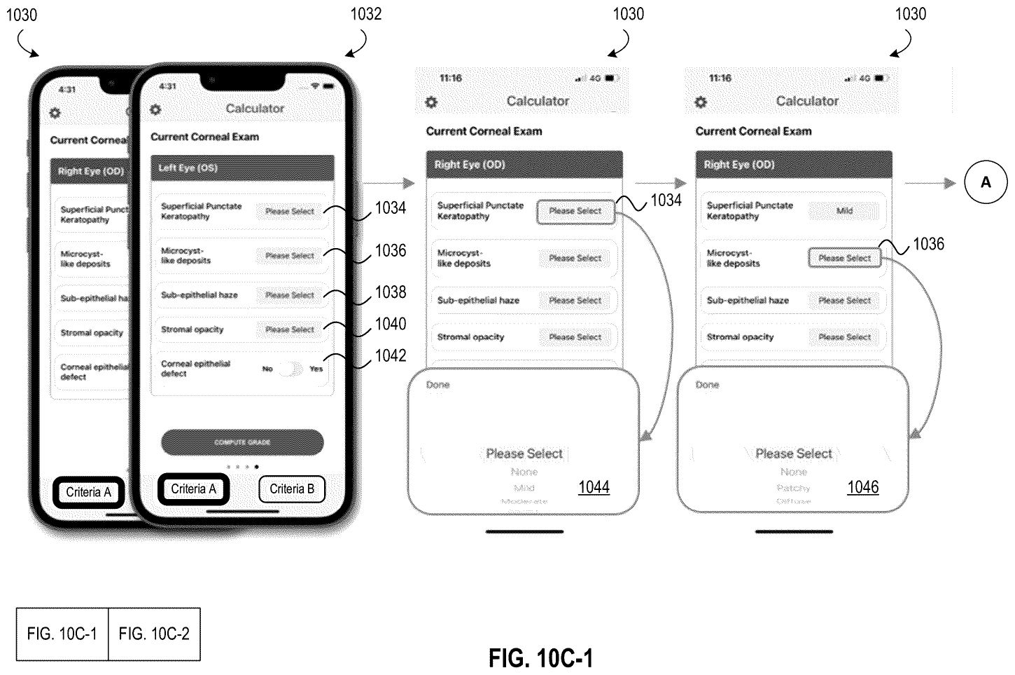

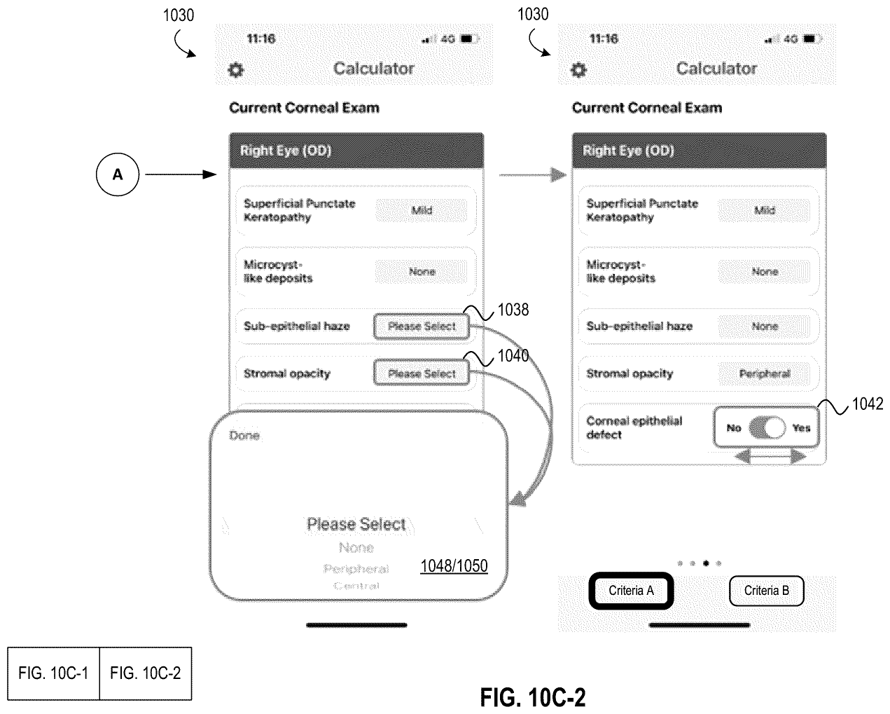

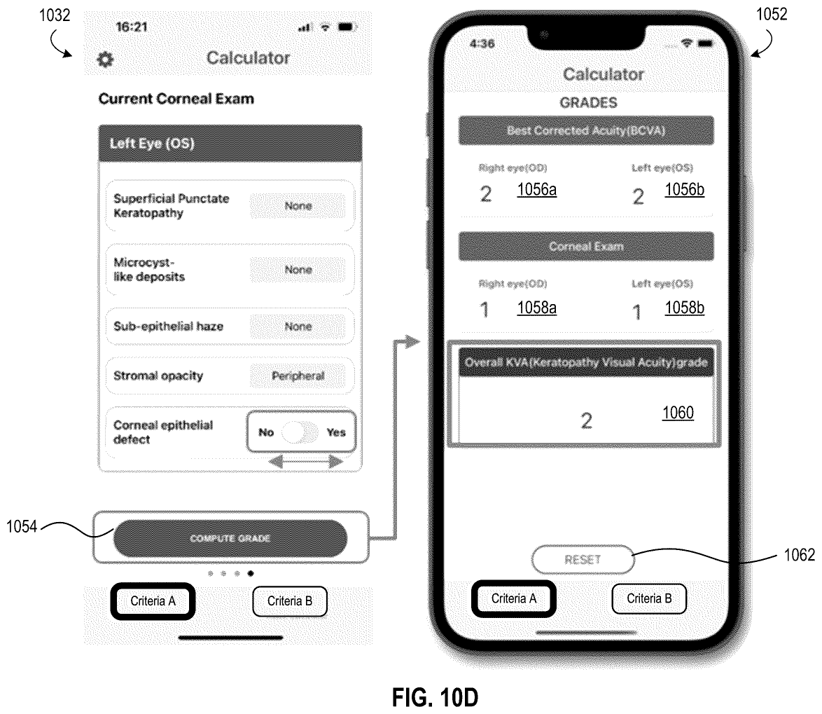

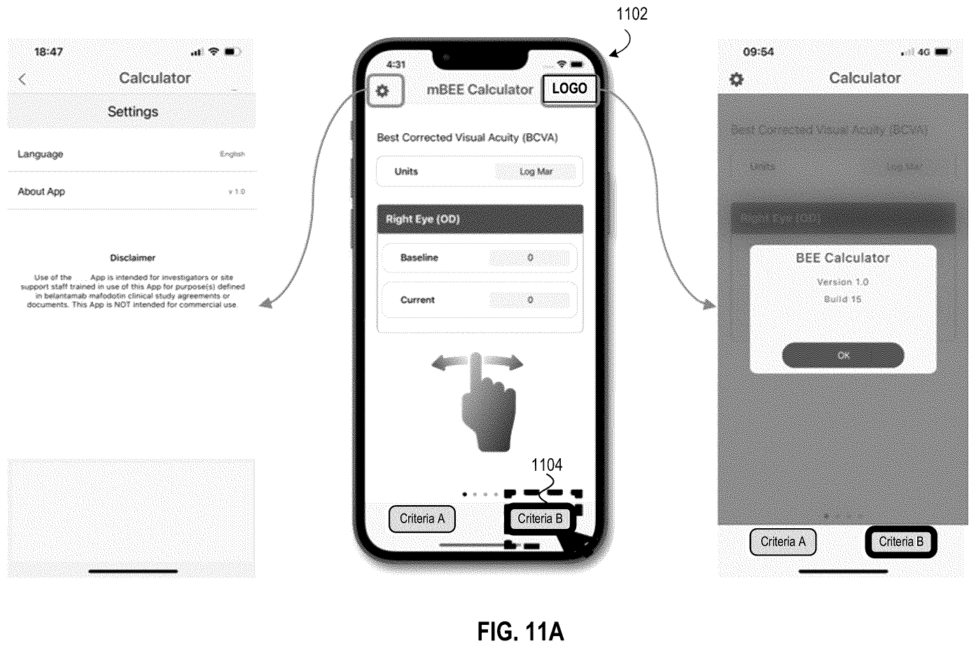

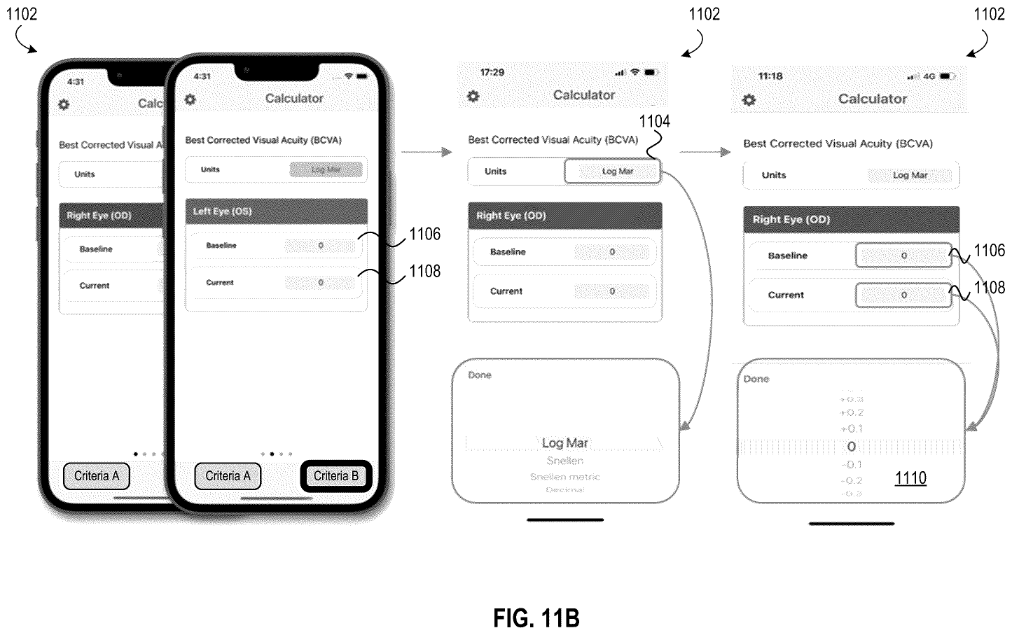

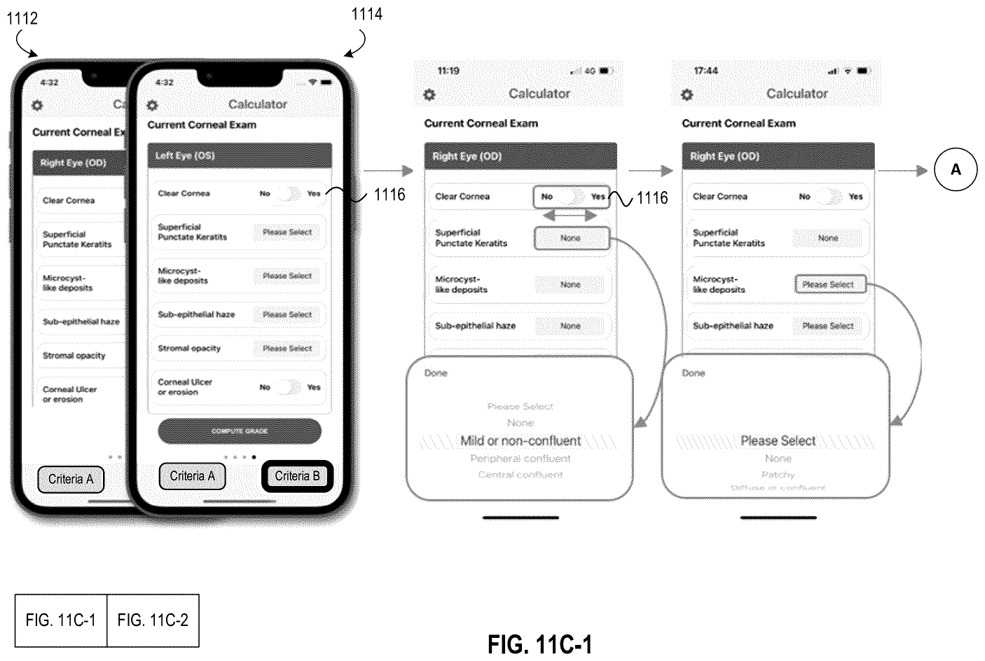

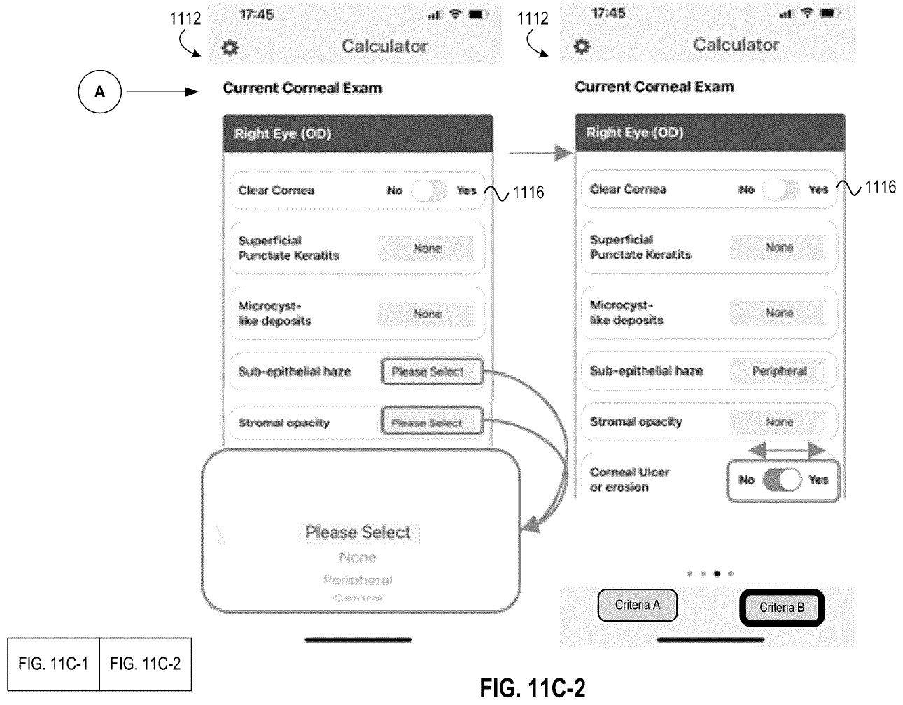

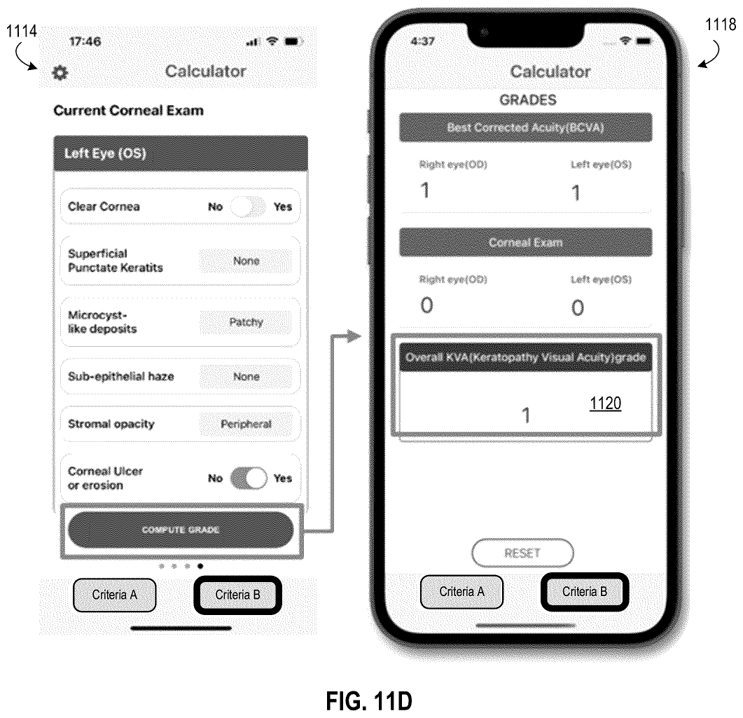

FIGS. 10A-D and FIGS. 11A-D are examples of screens of alternative GUIs of an ocular toxicity mitigation application. As described above, an ocular toxicity mitigation application may be configured to toggle between multiple (e.g., two) different sets of corneal assessment criteria. In this regard, an ocular toxicity mitigation application may include respective GUIs for each set of corneal assessment criteria. For example, one set of corneal assessment criteria may correspond to DreaMM14 (Study 209628) which utilize a modified KVA (mKVA) scale. As such, the KVA grade determined by an ocular toxicity mitigation application based on corneal assessment criteria that utilizes the mKVA scale may be referred to as an mKVA grade. Other example sets of corneal assessment criteria may also utilize the mKVA scale (e.g., future studies), and an ocular toxicity mitigation application may also include a GUI corresponding to one or more of those other sets of corneal assessment criteria. As described in further detail below, each GUI corresponding to a set of corneal assessment criteria may include multiple screens for which the user must input data (e.g., BCVA data, corneal assessment data) in order to calculate a KVA grade and/or an mKVA grade. The example GUIs described below with reference to FIGS. 10A-D and FIGS. 11A-D each include five screens: two screens for obtaining the BCVA data for a patient's right and left eye respectively, two screens for obtaining the corneal assessment data for the patient's right and left eye respectively, and one screen for displaying the KVA grade or mKVA grade automatically determined by the ocular toxicity mitigation application based on the BCVA data and corneal assessment data obtained. In other implementations of an ocular toxicity mitigation application, the GUIs may include more or fewer screens. Within the ocular toxicity mitigation application, a user may navigate (move) between screens using a swipe gesture (e.g., by swiping left and swiping right). Additional and/or alternative means for navigating between screens of the ocular toxicity mitigation application may be employed, e.g., additional and alternative touch gestures using one or more points of contact (e.g., one or more fingers or implement such as a stylus), user interface elements (e.g., buttons) that navigate to a different screen of the GUI when selected, voice input, eye-tracking input, and the like. In some example implementations, an ocular toxicity mitigation application may not save any patient data, e.g., the BCVA data and/or the corneal assessment data. As such, an ocular toxicity mitigation application may not require any patient identifiers and may be independent of any particular study, country, site, or visit.

Turning now to FIG. 10A, example screens 1002-1004 of an alternative GUI of an ocular toxicity mitigation application is shown. Screen 1002, in this example, is a settings screen which the user may display by selecting the cogwheel icon 1008 on screen 1004. The settings of the ocular toxicity mitigation application, in this example, include the current language being used (e.g., “English”). In some instances, an ocular toxicity mitigation application may be configured to use multiple different languages, and a user may select a different language by navigating to a settings screen (e.g., screen 1002) and selecting the desired language. Screen 1004, in this example, also allows the user to display a pop-up 1006 that includes information about the ocular toxicity mitigation application—e.g., the version and/or build of the ocular toxicity mitigation application that is currently installed at the user's mobile device-by selecting a logo button 1010 on screen 1004. Screen 1004, in this example, also includes two buttons 1012 (“Criteria A”) and 1012 (“Criteria B”) that allow the user to select and toggle between the GUIs corresponding to the different corneal assessment criteria the ocular toxicity mitigation application is configured to utilize. In this example, the ocular toxicity mitigation application is configured to utilize two different corneal assessment criteria, and the user may select and toggle between the two by selecting one of the buttons 1012 or 1014. In this example, the user has selected button 1012 (“Criteria A”) as indicated by way of example from its relatively thicker border.

In FIG. 10B, example screens 1016 and 1018 of an alternative GUI for receiving user input indicating selection of visual acuity assessment options are shown. Screen 1016, in this example, is configured to obtain visual acuity assessment data for the patient's right eye. Screen 1018, in this example, is configured to obtain visual acuity assessment data for the patient's left eye. In order to provide an additional cue to the user, increase ease of use, and avoid potential data entry errors, the headings of the screens configured to receive the visual acuity assessment data may be color coded. For example, screen 1016 for the patient's right eye may be color coded a first color (e.g., magenta) and screen 1018 for the patient's left eye may be color coded a second color (e.g., blue). Similar to the screens described above with reference to FIGS. 4A-B, screens 1016 and 1018 are configured to obtain visual acuity assessment data indicating a patient's baseline BCVA and the patient's current BCVA. The patient's baseline BCVA may be pre-populated from the patient's medical records that indicate the results of a previous ophthalmic examination. To enter the BCVA data, the user may first select the BCVA units 1020 to be used. Selecting the BCVA units 1020 may display a scrollable list of the available BCVA units 1022 (e.g., Snellen, Snellen metric, Decimal, logMar, etc.). The user may then select the desired BCVA unit from the list of the available BCVA units 1022. The screen for the patient's other eye (e.g., screen 1018 for the patient's left eye) may be pre-populated with the selected BCVA unit. In some examples, the user may only be allowed to change the BCVA units from one of the screens corresponding to one of the patient's eyes (e.g., screen 1016 for the patient's right eye). In some examples, a default BCVA unit (e.g., Snellen) may be pre-selected. With a BCVA unit selected, the user may enter the BCVA measurements for the patient by respectively selecting the baseline BCVA assessment 1024 and the current BCVA assessment 1026. Selecting the baseline BCVA assessment 1024 and selecting the currency BCVA assessment 1026 may display a scrollable list of the available BCVA measurements 1028 (e.g., 20/25, 20/20, etc.). The user may then select the desired BCVA measurement from the list of the available BVCA measurements 1028. The user may navigate between screens 1016 and 1018 to select the baseline BCVA measurement and the current BCVA measurement for each of the patient's right and left eyes respectively. The user may select the baseline BCVA and the current BCVA of the patient in order for the ocular toxicity mitigation application to calculate any change to the patient's BCVA since the last ophthalmic examination.

In FIG. 10C-1 and FIG. 10C-2, example screens 1030 and 1032 of an alternative GUI for receiving user input indicating selection of corneal assessment options are shown. Screen 1030, in this example, is configured to obtain corneal assessment data for the patient's right eye. Screen 1032, in this example, is configured to obtain corneal assessment data for the patient's left eye. In order to provide an additional cue to the user, increase ease of use, and avoid potential data entry errors, the headings of the screens configured to receive the corneal assessment data may be color coded. For example, screen 1030 for the patient's right eye may be color coded a first color (e.g., magenta) and screen 1032 for the patient's left eye may be color coded a second color (e.g., blue). Similar to the screens described above with reference to FIGS. 5A-B, screens 1030 and 1032 are configured to obtain corneal assessment data indicating the current health of the patient's cornea. The corneal assessment data, therefore, corresponds to the health of the patient's cornea at the time of current ophthalmic examination of the patient. The user may enter the corneal assessments for the patient by respectively selecting the corneal assessments of the selected criteria (e.g., “Criteria A”). In the example shown in FIG. 10C-1 and FIG. 10C-2, the corneal assessments include whether the patient has any superficial punctate keratopathy, any microcyst-like deposits, any sub-epithelial haze, any stromal opacity, and any corneal epithelial defect. Selecting the superficial punctate keratopathy assessment 1034 may display a scrollable list of the available superficial punctate keratopathy measurements 1044 (e.g., “None,” “Mild,” “Moderate,” etc.). Selecting the microcyst-like deposits assessment 1036 may display a scrollable list of the available microcyst-like deposits measurements 1046 (e.g., “None,” “Patchy,” “Diffuse,” etc.). Selecting the sub-epithelial haze assessment 1038 may display a scrollable list of sub-epithelial haze measurements 1048 (e.g., “None,” “Peripheral,” “Central,” etc.). Selecting the stromal opacity assessment 1040 may display a scrollable list of stromal opacity measurements 1050 (e.g., “None,” “Peripheral,” “Central,” etc.). As seen in FIG. 10C-1 and FIG. 10C-2, the user may be able to select a measurement for the corneal epithelial haze assessment 1042 by toggling a switch (e.g., “Yes” or “No”) although other types of user interface elements may be employed including those described herein (e.g., radio buttons, a scrollable list, etc.). The corneal epithelial defect includes whether the patient is exhibiting any corneal erosion and/or corneal ulcer. The presence of a corneal epithelial defect is a KVA grade 4 event according to some criteria.

In FIG. 10D, example screens 1032 and 1052 of an alternative GUI for receiving user input indicating selection of corneal assessment options and presenting a keratopathy visual acuity grade are shown. Screen 1032, in this example, further includes a user interface element 1054 (e.g., a button labeled “Compute Grade”) that the user may select to compute the KVA grade based on the visual acuity assessments and the cornel assessments that the user selected. In some instances, the ocular toxicity mitigation application may be configured to prevent the user from computing the KVA grade unless and until the user has selected a measurement for all visual acuity assessments and corneal assessments. In some instances, an ocular toxicity mitigation application may display a pop-up prompting the user to select a measurement for any missing visual acuity or corneal assessments. The user may swipe back and forth between the visual acuity assessment screens and the corneal assessment screens to double check the selected measurements and/or select measurements for any visual acuity or corneal assessments that are incomplete. Selecting the user interface element 1054 may cause the ocular toxicity mitigation application to calculate one or more grades for the ophthalmic examination of the patient. As seen in FIG. 10D and described above, for example, the ocular toxicity mitigation application is configured to calculate individual BCVA grades 1056a and 1056b respectively representing any changes in the BCVA for the patient's right and left eyes, individual corneal assessment grades 1058a and 1058b respectively representing the corneal health of the patients right and left eyes, and an overall KVA grade 1060 for the patient. Depending on the grading scale employed, the overall KVA grade may be based on (e.g., the same as) the highest or lowest grade of the BCVA grades 1056a-b and the corneal assessment grades 1058a-b. For example, one grading scale may grade BCVA and corneal health on a scale of 1-4 with 1 being the “best” grade and 4 being the “worst” grade. As shown by way of example in FIG. 10D, the worst grade of the BCVA grades and corneal assessment grades is 2 thus resulting in an overall KVA grade of 2. Other grading scales may be employed, e.g., a scale of 1-4 with 4 being the “best” grade and 1 being the “worst” grade. A user can clear the selected visual acuity assessments and the corneal health assessments by selecting a user interface element 1062 (e.g., a button labeled “Reset”) which may return the ocular toxicity mitigation application to its default state.

FIGS. 11A-D example screens of an alternative GUI of an ocular toxicity mitigation application are shown. The example screens shown in FIGS. 11A-D are similar to those shown in FIGS. 10A-D and discussed above. The example screens shown in FIGS. 11A-D, however, correspond to alternative criteria that the ocular toxicity mitigation application may be configured to utilize. As seen in screen 1102 of FIG. 11A, for example, the user has selected button 1104 (“Criteria B”) as indicated by way of example from its relatively thicker border. The disclosures provided above for the screens shown in FIGS. 10A-D also apply to the same elements of the screens shown in FIGS. 11A-D. As such, the following disclosures address only the differences between the respective screens. In the visual acuity assessment screen 1102 for the patient's right eye shown in FIG. 11B, the default BCVA unit 1104, in this example, is logMAR. Like above, with a BCVA unit selected, the user may enter the BCVA measurements for the patient by respectively selecting the baseline BCVA assessment 1106 and the current BCVA assessment 1108. Selecting the baseline BCVA assessment 1106 and selecting the currency BCVA assessment 1108 may display a scrollable list of the available BCVA measurements 1110 (e.g., Log Mar values of 0, ±0.1, ±0.2, ±0.3). The user may then select the desired BCVA measurement from the list of the available BVCA measurements 1110. A default BCVA measurement may also be pre-selected for each of the patient's eyes (e.g., a Log Mar value of 0). The respective corneal assessment screens 1112 and 1114 for the patient's right and left eyes shown in FIG. 11C-1 and 11C-2 further include a corneal assessment option 1116 to initially indicate whether the patient has a clear cornea. The corneal assessment option 1116, in this example, is a switch the user can toggle (e.g., between “Yes” and “No”). An ocular toxicity application may be configured to make the subsequent corneal assessment options editable depending on whether the user indicates the patient has a clear cornea. For example, if the user indicates the patient has a clear cornea (e.g., “Yes”), then the ocular toxicity application may prevent the user from selecting any subsequent corneal assessment options (e.g., by “graying out” those corneal assessment options or otherwise indicating they are not editable by the user). If, however, the user indicates the patient does not have a clear cornea (e.g., “No”), then the ocular toxicity application may allow the user to select corneal assessment measurements as described above. In screen 1118 shown in FIG. 11D, the overall KVA grade 1120 (e.g., an mKVA grade) for the alternative criteria (e.g., “Criteria B”) may be displayed. As seen in FIG. 11D, the BCVA grades, corneal assessment grades, and KVA grade in this example are based on a grading scale of 0-1 with 0 being the “best” grade and 1 being the “worst” grade.

It will be appreciated that, by configuring an ocular toxicity mitigation application to utilize different, selectable criteria for the visual acuity and corneal health assessments, users can advantageously pivot between such criteria depending on which criteria is employed by any particular site. If the patient undergoes any other treatments or procedures (e.g., cataract surgery) during the B-cell disorder therapy that would be expected to change the patient's baseline BCVA, the patient's baseline BCVA may need to be reevaluated after the BCVA stabilizes and prior to any further administration of the B-cell disorder therapy in order to “re-baseline” the patient. The user may then select the patient's new baseline BCVA as described above for subsequent ophthalmic examinations. In some instances, changes to the patient's BCVA may be deemed to be the result of other eye conditions and/or eye-related events (e.g., worsening cataracts, eye trauma, eye surgery, etc.) rather than due to the B-cell disorder treatment. In these instances, the user may omit selections for the patient's BCVA measurements (or select a BCVA measurement of “Not Applicable”—“NA”) for a given eye's BCVA. In these instances, that BCVA measurement may not be considered in the calculation of the overall KVA/mKVA grade in which case the overall KVA/mKVA grade may be based, for example, only based on the corneal assessments. It will also be appreciated that users of the ocular toxicity mitigation application who provide the visual acuity assessment data and the corneal health assessment data may broadly include a variety of different individuals including, but not limited to, the patients themselves as well as medical professionals such as prescribers of the B-cell disorder therapy and/or other medications/treatments, ophthalmologists, optometrists, administrators, and/or other individuals that are associated with (e.g., have been authorized to enter such data on behalf of) such medical professionals (e.g., nurses, assistants, etc.).

### Dosing and Dosing Regimens

Combination therapies may also be administered to treat patients newly diagnosed with multiple myeloma. In some examples, the patients may be ineligible for autologous stem cell transplant. In some examples, a second combination therapy is administered to the treated patients as maintenance therapy.

In some examples, as described in more detail below, a patient is treated with a combination therapy comprising a BCMA antigen binding protein, e.g., belantamab mafodotin; a proteasome inhibitor, e.g., bortezomib; an immunomodulatory imide drug (IMiD), e.g., lenalidomide; and a corticosteroid, e.g., dexamethasone on a three-week cycle until cycle eight (“induction treatment”). In some examples, such treatment is followed by a combination therapy comprising the BCMA antigen binding protein, the IMiD, and the corticosteroid on a four-week cycle thereafter (“maintenance treatment”). In some examples, belantamab mafodotin is administered in combination with bortezomib, lenalidomide, and dexamethasone (“VRd”) every three weeks (Q3W), every six weeks (Q6W), or every nine weeks (Q9W) to Cycle 8, and then in combination with lenalidomide and dexamethasone (“Rd”) every Q4W, every eight weeks (Q8W), or every 12 weeks (Q12W) thereafter.

In some examples, a recommended dose of belantamab mafodotin is 2.5 mg/kg of actual body weight given as an intravenous infusion over approximately 30 minutes once every 3 weeks until disease progression or unacceptable toxicity.

In some examples, a recommend dose reduction for adverse reactions is a 1.9 mg/kg dose of belantamab mafodotin administered intravenously once every 3 weeks.

In some examples, a patient is treated with a 1.9 mg/kg dose of belantamab mafodotin (Q3/4W). In some examples, a patient is treated with 1.9 mg/kg on Day 1 of each cycle.

In some examples, a patient is treated with a 1.4 mg/kg dose of belantamab mafodotin (Q6/8W). In some examples, a patient is treated with 1.4 mg/kg on Day 1 of every other cycle.

In some examples, a patient is treated with a 1.0 mg/kg dose of belantamab mafodotin Q3/4W. In some examples, a patient is treated with 1.0 mg/kg on Day 1 of each cycle.

In some examples, a patient is treated with a 1.4 mg/kg dose of belantamab mafodotin Q3/4W. In some examples, a patient is treated with 1.4 mg/kg on Day 1 of every other cycle.

In some examples, a patient is treated with a 1.4 mg/kg dose of belantamab mafodotin on Day 1 of cycle 1, then 1.0 mg/kg on Day 1 of every third cycle from cycle 4.

In some examples, a patient is treated with a 1.9 mg/kg dose of belantamab mafodotin of cycle 1, then 1.4 mg/kg on Day 1 of every third cycle from cycle 4.

In some examples, a patient is treated with a 1.9 mg/kg dose of belantamab mafodotin on Day 1 of cycle 1 and cycle 4, then 1.4 mg/kg on Day 1 of every third cycle from cycle 7.

In some examples, a patient is treated with a 1.4 mg/kg dose of belantamab mafodotin on Day 1 of cycle 1 and cycle 3, then 1.0 mg/kg on Day 1 of every third cycle from cycle 6.

In some examples, a patient is treated with a 1.0 mg/kg dose of belantamab mafodotin on Day 1 of cycle 1 and cycle 5, then 1.0 mg/kg on day 1 of every third cycle from cycle 9.

In some examples, a patient is treated with 1.9 milligram/kilogram (mg/kg) three-weekly (Q3W) dose of belantamab mafodotin intravenously (IV) on Day 1 of every 21-day cycle for the first 8 cycles in combination with VRd (bortezomib, lenalidomide, and dexamethasone, administered as described below). From cycle 9 onwards, the patient is treated with 1.9 mg/kg four-weekly (Q4W) dose of belantamab mafodotin intravenously on Day 1 of every 28-day cycle in combination with Rd (lenalidomide and dexamethasone, administered as described below).

In some examples, a patient is treated with 1.4 mg/kg six-weekly (Q6W) dose of belantamab mafodotin intravenously on Day 1 of every other 21-day cycle for the first 8 cycles in combination with VRd. From cycle 9 onwards, the patient is treated with 1.4 mg/kg eight-weekly (Q8W) dose of belantamab mafodotin intravenously on Day 1 of every other 28-day cycle in combination with Rd.

In some examples, a patient is treated with 1.9 mg/kg Q6W dose of belantamab mafodotin intravenously on Day 1 of every other 21-day cycle for the first 8 cycles in combination with VRd. From cycle 9 onwards, the patient is treated with 1.9 mg/kg Q8W dose of belantamab mafodotin intravenously on Day 1 of every other 28-day cycle in combination with Rd.

In some examples, a patient is treated with 1.0 mg/kg Q3W dose of belantamab mafodotin intravenously on Day 1 of every 21-day cycle for the first 8 cycles in combination with VRd. From cycle 9 onwards, the patient is treated with 1.0 mg/kg Q4W dose of belantamab mafodotin intravenously on Day 1 of every 28-day cycle in combination with Rd.

In some examples, a patient is treated with 1.4 mg/kg Q3W dose of belantamab mafodotin intravenously on Day 1 of every 21-day cycle for the first 8 cycles in combination with VRd. From cycle 9 onwards, the patient is treated with 1.4 mg/kg Q4W dose of belantamab mafodotin intravenously on Day 1 of every 28-day cycle in combination with Rd.

In some examples, a patient is treated with either 1.9 mg/kg or 2.5 mg/kg Q9W dose of belantamab mafodotin intravenously on Day 1 of every third 21-day cycle for the first 8 cycles in combination with VRd. From cycle 9 onwards, the patient is treated with either 1.9 mg/kg or 2.5 mg/kg Q12W dose of belantamab mafodotin intravenously on Day 1 of every third 28-day cycle in combination with Rd.

In some examples, a patient is treated with a total dose of either 1.9 mg/kg or 2.5 mg/kg of belantamab mafodotin intravenously (split in to two equal doses of 0.95 mg/kg or 1.25 mg/kg to be given on Day 1 and Day 8) Q6W of every other 21-day cycle for the first 8 cycles in combination with VRd. From cycle 9 onwards, the patient is treated with either 1.9 mg/kg or 2.5 mg/kg of belantamab mafodotin intravenously (split in to two equal doses of 0.95 mg/kg or 1.25 mg/kg to be given on Day 1 and Day 8) Q8W of every other 28-day cycle in combination with Rd.

In some examples, a patient is treated with 2.5 mg/kg Q6W dose of belantamab mafodotin intravenously on Day 1 of every other 21-day cycle for the first 8 cycles in combination with VRd. From cycle 9 onwards, the patient is treated with 2.5 mg/kg Q8W dose of belantamab mafodotin intravenously on Day 1 of every other 28-day cycle in combination with Rd.

Specifications for belantamab mafodotin treatment are shown in Table 6 below.

TABLE 6
Specifications for belantamab mafodotin treatment.

| | |
|---|---|
| Dosage form | Powder for solution for infusion |
| Unit dose | 100 mg/0.75 mg/kg |
| strength/Dose level | 100 mg/1.0 mg/kg |
| | 100 mg/1.4 mg/kg |
| | 100 mg/1.9 mg/kg |
| Route/Administration/ | Intravenous infusion/30 min to 1 hour |
| Duration | |
| Dosing instructions | Reconstitute belantamab mafodotin for |
| | injection, 100 mg with 2.0 mL of sterile water |
| | for injection, diluted with saline before use. |
| | Dilute belantamab mafodotin in normal 0.9% |
| | saline to the appropriate concentration for the |
| | dose. Doses of belantamab mafodotin are |
| | administrated as an IV infusion |
| | via an infusion pump. |

In some embodiments, when administered on Day 1, belantamab mafodotin is administered as the first agent in the clinic (prior to VRd or Rd) as a full single dose on Day 1 for 30-60 minute infusion, followed by a 1 to 2 hour rest period. The bortezomib dose is administered approximately 1 hour after belantamab mafodotin infusion is complete.

For patients treated on Day 1 of every cycle (Q3/4W), if a planned dose of belantamab mafodotin is held/missed for any reason, the next dose can be administered at Day 1 of the next planned 21-day or 28-day cycle, as long as the interval between 2 consecutive doses is at least 21 (±3) days or 28 (±3) days, respectively.

For patients treated on Day 1 of every other cycle (Q6/8W), if a planned dose of belantamab mafodotin is held/missed for any reason, the next dose can be administered at Day 1 of the next planned 21-day or 28-day cycle, as long as the interval between 2 consecutive doses is at least 42 (±3) days or 56 (±3) days, respectively.

For patients treated on Day 1 of every third cycle (Q9/12W), if a planned dose of belantamab mafodotin is held/missed for any reason, the next dose can be administered at Day 1 of the next planned 21-day or 28-day cycle, as long as the interval between 2 consecutive doses is at least 63 (±3) days or 84 (±3) days, respectively.

The belantamab mafodotin dose for both induction and maintenance treatment in combination with VRd is based on actual body weight calculated at baseline (Cycle 1 Day 1). If the change in body weight is greater than 10%, the dose can be recalculated based on the actual body weight at the time of dosing.

Various dosing schedules may be employed. In some examples, the dosing schedule comprises a total of 8 Cycles of belantamab mafodotin+VRd induction treatment followed by belantamab mafodotin+doublet Rd maintenance treatment.

Induction Treatment (Q3W)-Belantamab Mafodotin+VRd; up to Cycle 8: Belantamab mafodotin is administered intravenously (IV) on Day 1, either every 21-day cycle, every other 21-day cycle, every third 21-day cycle, or every fourth 21-day cycle for the first 8 (induction) Cycles in combination with VRd. Bortezomib is administered at a dose of 1.3 mg/m2 subcutaneously (SC) on Days 1, 4, 8, and 11 of every 21-day cycle for the eight induction Cycles. Lenalidomide is administered at a dose of 25 mg, orally, on Days 1-14 of a 21-day cycle for the eight induction Cycles. In patients with eGFR 30-60 mL/min/1.73 m² lenalidomide is administered at 10 mg daily on Days 1-14 of each 21-day cycle. In patients whose renal function improves during treatment to eGFR >60 mL/min/1.73 m² (confirmed, e.g., over two measurements two weeks apart), the dose of lenalidomide can be increased accordingly based on the Investigator judgement. Dexamethasone is administered at 20 mg, orally, on Days 1, 2, 4, 5, 8, 9, 11, and 12 of a 21-day cycle for the eight induction Cycles. Dexamethasone is taken at the same time of the day and may be taken at home. For patients who are older than 75 years, underweight (body mass index (BMI)<18.5), have poorly controlled diabetes mellitus, or prior intolerance/AE to steroid therapy, the dexamethasone dose may be administered at a dose of 10 mg on the Days indicated above.

Maintenance Treatment (Q4W): Belantamab Mafodotin+Rd; Cycle 9 Onward: In some examples, after the first 8 induction Cycles, belantamab mafodotin in combination with Rd is administered as maintenance treatment. Bortezomib is not administered as part of maintenance treatment. Belantamab mafodotin is administered intravenously (IV) on Day 1, either every 28-day cycle, every other 28-day cycle or every third 28-day cycle in combination with Rd. Lenalidomide is administered at a dose of 25 mg orally on Days 1-21 of each 28-day cycle until PD or unacceptable toxicity. In patients with eGFR 30-60 mL/min/1.73 m² lenalidomide is administered at 10 mg daily on Days 1-21 of each 28-day cycle. In patients whose renal function improves during treatment to eGFR >60 mL/min/1.73 m² (confirmed over 2 measurements 2 weeks apart), the dose of lenalidomide can be increased accordingly based on the Investigator judgement. Dexamethasone is administered at 40 mg, orally, on Days 1, 8, 15 and 22 of a 28-day cycle from Cycle 9 onward, until PD or unacceptable toxicity. Dexamethasone is taken at the same time of the day and may be taken at home. For patients who are older than 75 years, underweight (BMI <18.5), have poorly controlled diabetes mellitus, or prior intolerance/AE to steroid therapy, the dexamethasone dose may be administered at a dose of 20 mg on the days indicated above. Independently, the dose of nirogacestat administered as part of any of the combination therapies described above can be, for example, at least about 50, 100, 150, 200, or 250 mg, administered once or twice daily. In some embodiments, 100 mg of nirogacestat is administered twice a day.

An example treatment of a patient may be as follows. A patient newly diagnosed with multiple myeloma and who is ineligible for autologous stem cell transplant is administered a combination therapy comprising: (a) a therapeutically effective dose of belantamab mafodotin; and (b) a standard of care treatment comprising (i) a therapeutically effective dose of a proteasome inhibitor; (ii) a therapeutically effective dose of an immunomodulatory imide drug; and (iii) a therapeutically effective amount of a corticosteroid. The combination therapy comprises (a) a 1.0 mg/kg dose of belantamab mafodotin on Day 1 of cycle 1 and cycle 5 and a 1.0 mg/kg dose of belantamab mafodotin on day 1 of every third cycle from cycle 9; (b) administration of bortezomib at a dose of 1.3 mg/m² subcutaneously (SC) on Days 1, 4, 8, and 11 of every 21-day cycle for eight induction Cycles; (c) administration of lenalidomide (1) at a dose of 25 mg, orally, on Days 1-14 of a 21-day cycle for eight induction Cycles or (2) at 10 mg daily on Days 1-14 of each 21-day cycle if the individual has eGFR 30-60 mL/min/1.73 m²; and (d) administration of dexamethasone orally at a dose of 20 mg on Days 1, 2, 4, 5, 8, 9, 11, and 12 of a 21-day cycle for eight induction Cycles.

Hereafter, various characteristics of example embodiments will be highlighted in a set of numbered clauses or paragraphs. These characteristics should not be interpreted as being limiting on the claimed subject matter, but are instead provided as merely highlighting various characteristics described herein, without suggesting a particular order of importance or relevancy of those characteristics.

Clause 1. A method of mitigating ocular toxicity comprising obtaining a first therapeutically effective dose of a component of a B-cell disorder therapy; receiving instructions for administering the first therapeutically effective dose to a patient, wherein the instructions comprise an instruction to perform one or more ophthalmic examinations during the B-cell disorder therapy; administering, in accordance with the instructions, the first therapeutically effective dose; performing, in accordance with the instructions and using a computing device, an ophthalmic examination of a patient that has received the first therapeutically effective dose, wherein the ophthalmic examination comprises: a visual acuity assessment; and a corneal assessment; obtaining a keratopathy visual acuity grade at least by: selecting, based on the visual acuity assessment and from a plurality of predefined and selectable visual acuity assessment options presented at a user interface of the computing device, at least one visual acuity assessment option indicating a visual acuity of the patient; selecting, based on the corneal assessment and from a plurality of predefined and selectable corneal assessment options presented at the user interface, at least one corneal assessment option indicating a corneal state of the patient; and causing determination, automatically by the computing device based on the at least one visual acuity assessment option selected and based on the at least one corneal assessment option selected, of the keratopathy visual acuity grade; and administering, to the patient based on the keratopathy visual acuity grade obtained, a second therapeutically effective dose of the component of the B-cell disorder therapy.

Clause 2. The method of clause 1, further comprising reading, using the computing device, machine-readable data included in the instructions; and navigating, based on the machine-readable data read by the computing device, to a computer-executable application configured to: display, at the user interface of the computing device, the plurality of predefined and selectable visual acuity assessment options and the plurality of predefined and selectable corneal assessment options; receive the user input indicating the selection of the at least one visual acuity assessment option and the user input indicating the selection of the at least one corneal assessment option; and automatically determine the keratopathy visual acuity grade.

Clause 3. The method of clause 2, wherein the machine-readable data comprises an optical label that encodes an address of the computer-executable application; the reading the machine-readable data comprises: scanning, using an optical scanner of the computing device, the optical label; and decoding the address; and the navigating to the computer-executable application comprises navigating to the address decoded from the optical label.

Clause 4. The method of any one of clauses 2-3, further comprising installing, at the computing device, the computer-executable application.

Clause 5. The method of any one of clauses 1-4, wherein determination of the keratopathy visual acuity grade comprises: determination of at least one visual acuity grade; determination of at least one corneal grade; and determination of the keratopathy visual acuity grade based on the visual acuity grade and based on the corneal grade.

Clause 6. The method of any one of clauses 1-5, wherein the B-cell disorder therapy is a combination therapy.

Clause 7. The method of clauses 1-6, wherein the first therapeutically effective dose of the component of the B-cell disorder therapy comprises a therapeutically effective dose of belantamab mafodotin.

Clause 8. The method of clauses 1-6, wherein the B-cell disorder therapy is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab.

Clause 9. The method of clauses 1-8, wherein selecting the at least one visual acuity assessment option comprises: selecting, for a first eye of the patient, a first visual acuity assessment option; and selecting, for a second eye of the patient, a second visual acuity assessment option.

Clause 10. The method of any one of clauses 1-9, wherein the plurality of predefined and selectable corneal assessment options comprise a plurality of predefined and selectable superficial keratopathy assessment options; and selecting the at least one corneal assessment option comprises: selecting, for a first eye of the patient, a first superficial keratopathy assessment option; and selecting, for a second eye of the patient, a second superficial keratopathy assessment option.

Clause 11. The method of any one of clauses 1-10, wherein the plurality of predefined and selectable corneal assessment options comprise, for each corneal event of a plurality of corneal events, a plurality of predefined and selectable corneal event options; selecting the at least one corneal assessment option comprises selecting, for each eye of the patient and for each corneal event of the plurality of corneal events, a corneal event option; and the plurality of corneal events comprise at least one of: microcyst-like deposits; sub-epithelial haze; stromal opacity; a corneal epithelial defect; or a corneal ulcer or corneal erosion.

Clause 12. The method of any one of clauses 1-11, further comprising selecting, from a plurality of predefined and selectable visual acuity unit options presented at the user interface, a visual acuity unit, wherein the plurality of predefined and selectable visual acuity assessment options presented at the user interface is based on the visual acuity unit selected.

Clause 13. The method of any one of clauses 1-12, wherein the second therapeutically effective dose is lower than the first therapeutically effective dose.

Clause 14. The method of any one of clauses 1-13, further comprising selecting, from a plurality of predefined and selectable corneal assessment criteria options presented at the user interface, a corneal assessment criteria option, wherein the plurality of predefined and selectable corneal assessment options presented at the user interface is based on the corneal assessment criteria option selected.

Clause 15. The method of any one of clauses 1-14, further comprising causing determination, automatically by the computing device based on the keratopathy visual acuity grade obtained, of a recommended modification to the first therapeutically effective dose; and causing presentation, by the computing device at the user interface, of the recommended modification to the first therapeutically effective dose.

Clause 16. The method of any one of clauses 1-15, further comprising causing determination, automatically by the computing device based on output of a machine learning model trained at least on ophthalmic examination data and configured to provide at least one confidence score for at least one modification to a dose of the component of the B-cell disorder therapy, of at least one recommended modification to the first therapeutically effective dose; and causing presentation, by the computing device at the user interface, of the at least one recommended modification to the first therapeutically effective dose and at least one corresponding confidence score for the at least one recommended modification to the first therapeutically effective dose.

Clause 17. The method of any one of clauses 1-16, wherein the plurality of predefined and selectable visual acuity assessment options and the plurality of predefined and selectable corneal assessment options are presented at different screens of the user interface.

Clause 18. A computing device for mitigating ocular toxicity comprising one or more processors; a user interface comprising: a plurality of predefined and selectable visual acuity assessment options; and a plurality of predefined and selectable corneal assessment options; and memory storing instructions that, when executed by the one or more processors, cause the computing device to: obtain a keratopathy visual acuity grade for a patient that has received a first therapeutically effective dose of a component of a B-cell disorder therapy at least by: receiving, based on a visual acuity assessment of the patient, first user input indicating a visual acuity of the patient, wherein the first user input comprises a selection of at least one visual acuity assessment option of the plurality of predefined and selectable visual acuity assessment options; receiving, based on a corneal assessment of the patient, second user input indicating a corneal state of the patient, wherein the second user input comprises a selection of at least one corneal assessment option of the plurality of predefined and selectable corneal assessment options; and determining, automatically based on the at least one visual acuity assessment option selected and based on the at least one corneal assessment option selected, the keratopathy visual acuity grade for the patient; and present, at the user interface, the keratopathy visual acuity grade obtained.

Clause 19. The computing device of clause 18, wherein the instructions, when executed by the one or more processors, further cause the computing device to: read machine-readable data included in the instructions; and navigate, based on the machine-readable data read, to a computer-executable application configured to: display, at the user interface of the computing device, the plurality of predefined and selectable visual acuity assessment options and the plurality of predefined and selectable corneal assessment options; receive the user input indicating the selection of the at least one visual acuity assessment option and the user input indicating the selection of the at least one corneal assessment option; and automatically determine the keratopathy visual acuity grade.

Clause 20. The computing device of clause 19, wherein the machine-readable data comprises an optical label that encodes an address of the computer-executable application; the instructions, when executed by the one or more processors, cause the computing device to read the machine-readable data at least by: scanning, using an optical scanner of the computing device, the optical label; and decoding the address; and the instructions, when executed by the one or more processors, cause the computing device to navigate to the computer-executable application at least by navigating to the address decoded from the optical label.

Clause 21. The computing device of any one of clauses 19-20, wherein the instructions, when executed by the one or more processors, further cause the computing device to install the computer-executable application.

Clause 22. The computing device of any one of clauses 18-21, wherein the instructions, when executed by the one or more processors, cause the computing device to determine the keratopathy visual acuity grade at least by: determining at least one visual acuity grade; determining at least one corneal grade; and determining the keratopathy visual acuity grade based on the visual acuity grade and based on the corneal grade.

Clause 23. The computing device of any one of clauses 18-22, wherein the B-cell disorder therapy is a combination therapy.

Clause 24. The computing device of any one of clauses 18-23, wherein the first therapeutically effective dose of the component of the B-cell disorder therapy comprises a therapeutically effective dose of belantamab mafodotin.

Clause 25. The computing device of any one of clauses 18-23, wherein the B-cell disorder therapy is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, rituximab or any combination therapy thereof.

Clause 26. The computing device of any one of clauses 18-25, wherein the instructions, when executed by the one or more processors, cause the computing device to receive selection of the at least one visual acuity assessment option at least by: receiving user input indicating selection, for a first eye of the patient, of a first visual acuity assessment option; and receiving user input indicating selection, for a second eye of the patient, of a second visual acuity assessment option.

Clause 27. The computing device of any one of clauses 18-26, wherein the plurality of predefined and selectable corneal assessment options comprise a plurality of predefined and selectable superficial keratopathy assessment options; and the instructions, when executed by the one or more processors, cause the computing device to receive selection of the at least one corneal assessment option at least by: receiving user input indicating selection, for a first eye of the patient, of a first superficial keratopathy assessment option; and receiving user input indicating selection, for a second eye of the patient, of a second superficial keratopathy assessment option.

Clause 28. The computing device of any one of clauses 18-27, wherein the plurality of predefined and selectable corneal assessment options comprise, for each corneal event of a plurality of corneal events, a plurality of predefined and selectable corneal event options; and the instructions, when executed by the one or more processors, cause the computing device to receive selection of the at least one corneal assessment option at least by: receiving user input indicating selection, for each eye of the patient and for each corneal event of the plurality of corneal events, of a corneal event option; and the plurality of corneal events comprise at least one of: microcyst-like deposits; sub-epithelial haze; stromal opacity; a corneal epithelial defect; or a corneal ulcer or corneal erosion.

Clause 29. The computing device of any one of clauses 18-28, wherein the user interface further comprises a plurality of predefined and selectable visual acuity units options; the instructions, when executed by the one or more processors, further cause the computing device to receive user input indicating a visual acuity unit, wherein the user input comprises a selection of the visual acuity unit from the plurality of predefined and selectable visual acuity unit options; and the plurality of predefined and selectable visual acuity assessment options presented at the user interface is based on the visual acuity unit selected.

Clause 30. The computing device of any one of clauses 18-29, wherein the keratopathy visual acuity grade indicates that a second therapeutically effective dose of the component of the B-cell disorder therapy should be lower than the first therapeutically effective dose.

Clause 31. The computing device of any one of clauses 18-30, wherein the user interface further comprises a plurality of predefined and selectable corneal assessment criteria options; the instructions, when executed by the one or more processors, further cause the computing device to receive user input indicating a corneal assessment criteria option, wherein the user input comprises a selection of the corneal assessment criteria option from the plurality of predefined and selectable corneal assessment criteria options; and the plurality of predefined and selectable corneal assessment options presented at the user interface is based on the corneal assessment criteria option selected.

Clause 32. The computing device of any one of clauses 18-31, wherein the instructions, when executed by the one or more processors, further cause the computing device to: automatically determine, based on the keratopathy visual acuity grade obtained, a recommended modification to the first therapeutically effective dose; and present, at the user interface, the recommended modification to the first therapeutically effective dose.

Clause 33. The computing device of any one of clauses 18-32, wherein the instructions, when executed by the one or more processors, further cause the computing device to: automatically determine, based on output of a machine learning model trained at least on ophthalmic examination data and configured to provide at least one confidence score for at least one modification to a dose of the component of the B-cell disorder therapy, at least one recommended modification to the first therapeutically effective dose; and present, at the user interface, the at least one recommended modification to the first therapeutically effective dose and at least one corresponding confidence score for the at least one recommended modification to the first therapeutically effective dose.

Clause 34. The computing device of any one of clauses 18-33, wherein the instructions, when executed by the one or more processors, further cause the computing device to present, at different screens of the user interface, the plurality of predefined and selectable visual acuity assessment options and the plurality of predefined and selectable corneal assessment options.

Clause 35. A method of mitigating ocular toxicity comprising providing a first therapeutically effective dose of a component of a B-cell disorder therapy; providing instructions for administering the first therapeutically effective dose to a patient, wherein the instructions comprise an instruction to perform one or more ophthalmic examinations during the B-cell disorder therapy, and wherein the instructions comprise machine-readable data that, when read by a computing device, cause the computing device to navigate to a computer-executable application configured to: provide, based on a visual acuity assessment of an ophthalmic examination and based on a corneal assessment of the ophthalmic examination, a keratopathy visual acuity grade at least by: receiving user input indicating a selection, from a plurality of predefined and selectable visual acuity assessment options presented at a user interface of the computing device, of at least one visual acuity assessment option indicating a visual acuity of the patient; receiving user input indicating a selection, from a plurality of predefined and selectable corneal assessment options presented at the user interface, of at least one corneal assessment option indicating a corneal state of the patient; and determine, automatically based on the at least one visual acuity assessment option selected and based on the at least one corneal assessment option selected, the keratopathy visual acuity grade; and present, at the user interface of the computing device, the keratopathy visual acuity grade determined.

Clause 36. The method of clauses 35, wherein the machine-readable data, when read by the computing device, causes the computing device to navigate to the computer-executable application at least by navigating to a data store that stores the computer-executable application and is configured to send the computer-executable application to the computing device based on a request received at the data store from the computing device.

Clause 37. The method of any one of clauses 35 and 36, wherein the machine-readable data comprises an optical label that encodes an address of the computer-executable application, and wherein the optical label, when optically read by the computing device, causes the computing device to decode the address of the computer-executable application.

Clause 38. The method of any one of clauses 35-37, wherein the computer-executable application is configured to be installed at the computing device.

Clause 39. The method of any one of clauses 35-38, wherein the computer-executable application is configured to determine the keratopathy visual acuity grade at least by: determining at least one visual acuity grade; determining at least one corneal grade; and determining the keratopathy visual acuity grade based on the visual acuity grade and based on the corneal grade.

Clause 40. The method of any one of clauses 35-39, wherein the B-cell disorder therapy is a combination therapy.

Clause 41. The method of any one of clauses 35-40, wherein the first therapeutically effective dose of the component of the B-cell disorder therapy comprises a therapeutically effective dose of belantamab mafodotin.

Clause 42. The method of any one of clauses 35-40, wherein the B-cell disorder therapy is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab.

Clause 43. The method of any one of clauses 35-42, wherein the computer-executable application is configured to receive the selection of the at least one visual acuity assessment option at least by: receiving selection, for a first eye of the patient, of a first visual acuity assessment option; and receiving selection, for a second eye of the patient, of a second visual acuity assessment option.

Clause 44. The method of any one of clauses 35-43, wherein the plurality of predefined and selectable corneal assessment options comprise a plurality of predefined and selectable superficial keratopathy assessment options; and the computer-executable application is configured to receive the selection of the at least one visual acuity assessment option at least by: receiving selection, for a first eye of the patient, of a first superficial keratopathy assessment option; and receiving selection, for a second eye of the patient, a second superficial keratopathy assessment option.

Clause 45. The method of any one of clauses 35-44, wherein the plurality of predefined and selectable corneal assessment options comprise, for each corneal event of a plurality of corneal events, a plurality of predefined and selectable corneal event options; and the computer-executable application is configured to receive the selection of the at least one corneal assessment option at least by receiving, for each eye of the patient and for each corneal event of the plurality of corneal events, a corneal event option; and the plurality of corneal events comprise at least one of: microcyst-like deposits; sub-epithelial haze; stromal opacity; a corneal epithelial defect; or a corneal ulcer or corneal erosion.

Clause 46. The method of any one of clauses 35-45, wherein the computer-executable application is further configured to receive user input indicating a selection, from a plurality of predefined and selectable visual acuity unit options presented at the user interface, of a visual acuity unit; and the plurality of predefined and selectable visual acuity assessment options presented at the user interface is based on the visual acuity unit selected.

Clause 47. The method of any one of clauses 35-46, wherein the keratopathy visual acuity grade indicates a second therapeutically effective dose of the component of the B-cell disorder therapy should be lower than the first therapeutically effective dose.

Clause 48. The method of any one of clauses 35-47, wherein the computer-executable application is further configured to receive user input indicating a selection, from a plurality of predefined and selectable corneal assessment criteria options presented at the user interface, a corneal assessment criteria option; and the plurality of predefined and selectable corneal assessment options presented at the user interface is based on the corneal assessment criteria option selected.

Clause 49. The method of any one of clauses 35-48, wherein the computer-executable application is further configured to: automatically determine, based on the keratopathy visual acuity grade obtained, a recommended modification to the first therapeutically effective dose; and present, at the user interface, the recommended modification to the first therapeutically effective dose.

Clause 50. The method of any one of clauses 35-49, wherein the computer-executable application is further configured to automatically determine, based on output of a machine learning model trained at least on ophthalmic examination data and configured to provide at least one confidence score for at least one modification to a dose of the component of the B-cell disorder therapy, at least one recommended modification to the first therapeutically effective dose; and present, at the user interface, the at least one recommended modification to the first therapeutically effective dose and at least one corresponding confidence score for the at least one recommended modification to the first therapeutically effective dose.

Clause 51. The method of any one of clauses 35-50, wherein the computer-executable application is further configured to present the plurality of predefined and selectable visual acuity assessment options and the plurality of predefined and selectable corneal assessment options are presented at different screens of the user interface.

Clause 52. A method of mitigating ocular toxicity comprising receiving, by a computing device, first B-cell disorder treatment data and first ophthalmic examination data, wherein: the first B-cell disorder treatment data comprises indication of one or more therapeutically effective doses of a component of a B-cell disorder therapy administered to one or more patients of a plurality of patients; and the first ophthalmic examination data comprises, for one or more patients of the plurality of patients, one or more visual acuity assessments and one or more corneal assessments; training, by the computing device based on the first B-cell disorder treatment data and based on the first ophthalmic examination data received, a machine learning model configured to provide one or more recommendations for administration of the component of the B-cell disorder therapy; receiving, by the computing device, second B-cell disorder treatment data associated with a patient being treated with the B-cell disorder therapy and second ophthalmic examination data associated with the patient, wherein: the second B-cell disorder treatment data comprises indication of one or more therapeutically effective doses of the component of the B-cell disorder therapy administered to the patient; and the second ophthalmic examination data comprises one or more visual acuity assessments of the patient and one or more corneal assessments of the patient; generating, by the computing device using the machine learning model, indication of at least one recommendation for administration of the component of the B-cell disorder therapy to the patient; and providing, by the computing device and to a medical provider associated with administration of the B-cell disorder therapy, the indication of the at least one recommendation for administration of the component of the B-cell disorder therapy.

Clause 53. The method of clauses 52, wherein the component of the B-cell disorder therapy comprises belantamab mafodotin.

Clause 54. The method of any one of clauses 52 and 53, wherein the first ophthalmic examination data comprises, for each patient of the plurality of patients, one or more keratopathy visual acuity grades; and each keratopathy visual acuity grade of the one or more keratopathy visual acuity grades is based on a visual acuity assessment and a corneal assessment.

Clause 55. The method of any one of clauses 52-54, further comprising generating, by the computing device using the machine learning model and for each recommendation of the at least one recommendation, a confidence score of the recommendation; and providing, by the computing device and to the medical provider, the confidence score for each corresponding recommendation of the at least one recommendation.

Clause 56. The method of any one of clauses 52-55, further comprising receiving, by the computing device, third ophthalmic examination data associated with an ophthalmic examination of the patient performed after a recommended dose has been administered to the patient in accordance with the at least one recommendation, wherein the third ophthalmic data comprises a visual acuity assessment of the patient and a corneal assessment of the patient; and retraining, by the computing device based on the recommended dose administered to the patient and based on the third ophthalmic data, the machine learning model.

Clause 57. The method of any one of clauses 52-56, further comprising receiving, by the computing device, patient data, wherein the patient data comprises indication of one or more characteristics of one or more patients of the plurality of patients, and wherein the training the machine learning model is further based on the patient data.

Clause 58. The method of clauses 57, wherein the one or more characteristics comprise at least one of a height of a patient; a weight of a patient; an age of a patient; a comorbidity of a patient; or a medical history of a patient.

Clause 59. The method of any one of clauses 52-58, wherein the one or more recommendations that the machine learning model is configured to provide comprise at least one of a recommended dosage modification of the component of the B-cell disorder therapy or a recommend dose of the component of the B-cell disorder therapy.

Clause 60. The method of any one of clauses 52 and 54-58, wherein the B-cell disorder therapy is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab.

Clause 61. A computer-implemented method comprising displaying, by a computing device at a user interface of the computing device and in association with an ophthalmic examination of a patient being treated for a B-cell disorder, a plurality of predefined and selectable visual acuity assessment options; displaying, by the computing device at the user interface and in association with the ophthalmic examination, a plurality of predefined and selectable corneal assessment options; receiving, by the computing device at the user interface and in association with a visual acuity assessment of the ophthalmic examination, first user input indicating selection, from the plurality of predefined and selectable visual acuity assessment options, of at least one visual acuity assessment option that indicates a visual acuity of the patient; receiving, by the computing device at the user interface and in association with a corneal assessment of the ophthalmic examination, second user input indicating selection, from the plurality of predefined and selectable corneal assessment options, of at least one corneal assessment option that indicates a corneal state of the patient; determining, automatically by the computing device based on the at least one visual acuity assessment option selected and based on the at least one corneal assessment option selected, a keratopathy visual acuity grade; and causing, by the computing device, output of the keratopathy visual acuity grade determined.

Clause 62. The computer-implemented method of clause 61, further comprising reading, by the computing device, machine-readable data included in instructions for administering a therapeutically effective dose of a component of a B-cell disorder therapy to the patient; and navigating, by the computing device based on the machine-readable data read by the computing device, to a computer-executable application configured to: display the plurality of predefined and selectable visual acuity assessment options and the plurality of predefined and selectable corneal assessment options; receive the first user input and the second user input; and determine the keratopathy visual acuity grade.

Clause 63. The computer-implemented method of clause 62, wherein the machine-readable data comprises an optical label that encodes an address of the computer-executable application; and the reading the machine-readable data comprises: scanning, using an optical scanner of the computing device, the optical label; and decoding the address; and the navigating to the computer-executable application comprises navigating to the address decoded from the optical label.

Clause 64. The computer-implemented method of any one of clauses 62-63, further comprising installing, by the computing device, the computer-executable application.

Clause 65. The computer-implemented method of any one of clauses 61-64, wherein the automatically determining the keratopathy visual acuity grade comprises determining at least one visual acuity grade; determining at least one corneal grade; and determining the keratopathy visual acuity grade based on the visual acuity grade and based on the corneal grade.

Clause 66. The computer-implemented method of clause 65, further comprising initiating the determining the at least one visual acuity grade based on a threshold quantity of visual acuity assessment options being selected; initiating the determining the at least one corneal grade based on a threshold quantity of corneal assessment options being selected; and initiating the determining the keratopathy visual acuity grade based on the at least one visual acuity grade being determined and the at least one corneal grade being determined.

Clause 67. The computer-implemented method of any one of clauses 61-66, further comprising initiating the determining the keratopathy visual acuity grade based on a threshold quantity of visual acuity assessment options being selected and based on a threshold quantity of corneal assessment options being selected.

Clause 68. The computer-implemented method of any one of clauses 61-67, wherein the receiving the first user input indicating selection of at least one visual acuity assessment option comprises receiving, for a first eye of the patient, user input indicating selection of a first visual acuity assessment option; and receiving, for a second eye of the patient, user input indicating selection of a second visual acuity assessment option.

Clause 69. The computer-implemented method of any one of clauses 61-68, wherein the plurality of predefined and selectable corneal assessment options comprise a plurality of predefined and selectable superficial keratopathy assessment options; and the receiving the second user input indicating selection of at least one corneal assessment option comprises: receiving, for a first eye of the patient, selection of a first superficial keratopathy assessment option; and receiving, for a second eye of the patient, selection of a second superficial keratopathy assessment option.

Clause 70. The computer-implemented method of any one of clauses 61-69, wherein: the plurality of predefined and selectable corneal assessment options comprise, for each corneal event of a plurality of corneal events, a plurality of predefined and selectable corneal event options; receiving the second user input indicating selection of at least one corneal assessment option comprises receiving, for each eye of the patient and for each corneal event of the plurality of corneal events, user input indicating selection of a corneal event option; and the plurality of corneal events comprise at least one of: microcyst-like deposits; sub-epithelial haze; stromal opacity; a corneal epithelial defect; or a corneal ulcer or corneal erosion.

Clause 71. The computer-implemented method of any one of clauses 61-70, further comprising: displaying, by the computing device at the user interface and in association with the ophthalmic examination, a plurality of predefined and selectable visual acuity unit options; receiving, by the computing device at the user interface, third user input indicating selection, from the plurality of predefined and selectable visual acuity unit options, of a visual acuity unit; and configuring, by the computing device and based on the visual acuity unit selected, the plurality of predefined and selectable visual acuity assessment options displayed.

Clause 72. The computer-implemented method of any one of clauses 61-71, further comprising displaying, by the computing device at the user interface and in association with the ophthalmic examination, a plurality of predefined and selectable corneal assessment criteria options; receiving, by the computing device at the user interface, third user input indicating selection, from the plurality of predefined and selectable corneal assessment criteria options, of a corneal assessment criteria option; and configuring, by the computing device and based on the corneal assessment criteria option selected, the plurality of predefined and selectable corneal assessment options displayed.

Clause 73. The computer-implemented method of any one of clauses 61-72, further comprising: determining, automatically by the computing device based on the keratopathy visual acuity grade obtained, a recommended modification to a therapeutically effective dose of a component of a B-cell disorder therapy associated with the patient; and causing, by the computing device, output of the recommended modification to the first therapeutically effective dose.

Clause 74. The computer-implemented method of any one of clauses 61-73, wherein causing output of the keratopathy visual acuity grade comprises sending, to a second computing device in signal communication with the computing device, the keratopathy visual acuity grade.

Clause 75. The computer-implemented method of any one of clauses 61-74, further comprising sending, by the computing device to a second computing device in signal communication with the computing device, ophthalmic examination data comprising at least one of: visual acuity assessment data associated with one or more visual acuity assessments of the patient; or corneal assessment data associated with one or more corneal assessments of the patient.

Clause 76. The computer-implemented method of any one of clauses 61, further comprising receiving, from a second computing device in signal communication with the computing device, at least one of: a recommended dose of a component of a B-cell disorder therapy associated with the patient; or a recommended modification to the dose of the component of the B-cell disorder therapy.

Clause 77. The computer-implemented method of clause 76, wherein the second computing device is configured to use at least one machine learning model that is trained at least on ophthalmic examination data and configured to determine at least one of: one or more recommended doses of the component of the B-cell disorder therapy; or one or more recommended modifications to the dose of the component of the B-cell disorder therapy

Clause 78. The computer-implemented method of clause 77, wherein the at least one machine learning model is further configured to determine at least one of: a confidence score for at least one recommended dose of the one or more recommended doses; or a confidence score for at least one recommended modification of the one or more recommended modifications.

Clause 79. The computer-implemented method of any one of clauses 61-78 wherein the displaying the plurality of predefined and selectable visual acuity assessment options comprises displaying the plurality of predefined and selectable visual acuity assessment options on a first screen of the user interface; and the displaying the plurality of predefined and selectable corneal assessment options comprises displaying the plurality of predefined and selectable corneal assessment options on a second screen of the user interface that is different than the first screen.

Clause 80. The computer-implemented method of any one of clauses 61-79, wherein a B-cell disorder therapy associated with the patient is a combination therapy.

Clause 81. The computer-implemented method of any one of clauses 61-80, wherein a therapeutically effective dose of a B-cell disorder therapy associated with the patient comprises a therapeutically effective dose of belantamab mafodotin.

Clause 82. The computer-implemented method of any one of clauses 61-80, wherein a B-cell disorder therapy associated with the patient is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab.

Clause 83. The computer-implemented method of any one of clauses 61-83, wherein the keratopathy visual acuity grade indicates a therapeutically effective dose of a component of a B-cell disorder therapy associated with the patient should be lower than a previously administered therapeutically effective dose of the component of the B-cell disorder therapy.

Clause 84. The computer-implemented method of any one of clauses 61-83, wherein causing output of the keratopathy visual acuity grade comprises displaying, at the user interface of the computing device, the keratopathy visual acuity grade determined.

Clause 85. The computer-implemented method of any one of clauses 61-85, wherein the first user input comprises first touch input received at a touchscreen of the computing device; and the second user input comprises second touch input received at the touchscreen.

Clause 86. A computing device comprising one or more processors; and memory storing instructions that, when executed by the one or more processors, cause the computing device to perform the method of any one of clauses 61-85.

Clause 87. A system comprising a first computing device configured to perform the method of any one of clauses 61-85; and a second computing device configured to receive, from the first computing device, at least one of ophthalmic examination data associated with the ophthalmic examination or the keratopathy visual acuity grade.

Clause 88. A non-transitory computer-readable medium storing instructions that, when executed, cause performance of the method of any one of clauses 61-85.

Clause 89. A system comprising a first computing device; and a second computing device; wherein the first computing device is configured to: display, at a user interface of the computing device and in association with an ophthalmic examination of a patient being treated for a B-cell disorder, a plurality of predefined and selectable visual acuity assessment options; display, at the user interface and in association with the ophthalmic examination, a plurality of predefined and selectable corneal assessment options; receive, at the user interface and in association with a visual acuity assessment of the ophthalmic examination, first user input indicating selection, from the plurality of predefined and selectable visual acuity assessment options, of at least one visual acuity assessment option that indicates a visual acuity of the patient; receive, at the user interface and in association with a corneal assessment of the ophthalmic examination, second user input indicating selection, from the plurality of predefined and selectable corneal assessment options, of at least one corneal assessment option that indicates a corneal state of the patient; determine, automatically based on the at least one visual acuity assessment option selected and based on the at least one corneal assessment option selected, a keratopathy visual acuity grade; and send, to the second computing device, the keratopathy visual acuity grade determined; wherein the second computing device is configured to: receive, from the first computing device, the keratopathy visual acuity grade determined; and send, to the first computing device, at least one of: a recommended dose of a component of a B-cell disorder therapy associated with the patient; or a recommended modification to the dose of the component of the B-cell disorder therapy.

Clause 90. The system of clause 89, wherein the first computing device is further configured to: read machine-readable data included in instructions for administering a therapeutically effective dose of the component of the B-cell disorder therapy to the patient; and navigate, based on the machine-readable data read by the computing device, to a computer-executable application configured to: display the plurality of predefined and selectable visual acuity assessment options and the plurality of predefined and selectable corneal assessment options; receive the first user input and the second user input; and determine the keratopathy visual acuity grade.

Clause 91. The system of clause 90, wherein: the machine-readable data comprises an optical label that encodes an address of the computer-executable application; and the first computing device is configured to: read the machine-readable data at least by: scanning, using an optical scanner of the computing device, the optical label; and decoding the address; and navigate to the computer-executable application at least by navigating to the address decoded from the optical label.

Clause 92. The system of clause 90, wherein the first computing device is further configured to install the computer-executable application.

Clause 93. The system of any one of clauses 89-92, wherein the first computing device is configured to automatically determine the keratopathy visual acuity grade at least by: determining at least one visual acuity grade; determining at least one corneal grade; and determining the keratopathy visual acuity grade based on the visual acuity grade and based on the corneal grade.

Clause 94. The system of any one of clauses 89-93, wherein the first computing device is further configured to: initiate determination of the at least one visual acuity grade based on a threshold quantity of visual acuity assessment options being selected; initiate determination of at least one corneal grade based on a threshold quantity of corneal assessment options being selected; and initiate determination of the keratopathy visual acuity grade based on the at least one visual acuity grade being determined and the at least one corneal grade being determined.

Clause 95. The system of any one of clauses 89-94, wherein the first computing device is further configured to initiate determination of the keratopathy visual acuity grade based on a threshold quantity of visual acuity assessment options being selected and based on a threshold quantity of corneal assessment options being selected.

Clause 96. The system of any one of clauses 89-95, wherein the first computing device is configured to receive the first user input indicating selection of at least one visual acuity assessment option at least by: receiving, for a first eye of the patient, user input indicating selection of a first visual acuity assessment option; and receiving, for a second eye of the patient, user input indicating selection of a second visual acuity assessment option.

Clause 97. The system of any one of clauses 89-96, wherein the plurality of predefined and selectable corneal assessment options comprise a plurality of predefined and selectable superficial keratopathy assessment options; and the first computing device is configured to receive the second user input indicating selection of at least one corneal assessment option at least by: receiving, for a first eye of the patient, selection of a first superficial keratopathy assessment option; and receiving, for a second eye of the patient, selection of a second superficial keratopathy assessment option.

Clause 98. The system of any one of clauses 89-97, wherein the plurality of predefined and selectable corneal assessment options comprise, for each corneal event of a plurality of corneal events, a plurality of predefined and selectable corneal event options; the first computing device is configured to receive the second user input indicating selection of at least one corneal assessment option at least by receiving, for each eye of the patient and for each corneal event of the plurality of corneal events, user input indicating selection of a corneal event option; and the plurality of corneal events comprise at least one of: microcyst-like deposits; sub-epithelial haze; stromal opacity; a corneal epithelial defect; or a corneal ulcer or corneal erosion.

Clause 99. The system of any one of clauses 89-98, wherein the first computing device is further configured to: display, at the user interface and in association with the ophthalmic examination, a plurality of predefined and selectable visual acuity unit options; receive, at the user interface, third user input indicating selection, from the plurality of predefined and selectable visual acuity unit options, of a visual acuity unit; and configure, based on the visual acuity unit selected, the plurality of predefined and selectable visual acuity assessment options displayed.

Clause 100. The system of any one of clauses 89-99, wherein the first computing device is further configured to: display, at the user interface and in association with the ophthalmic examination, a plurality of predefined and selectable corneal assessment criteria options; receive, at the user interface, third user input indicating selection, from the plurality of predefined and selectable corneal assessment criteria options, of a corneal assessment criteria option; and configure, based on the corneal assessment criteria option selected, the plurality of predefined and selectable corneal assessment options displayed.

Clause 101. The system of any one of clauses 89-100, wherein the first computing device is further configured to: determine, automatically based on the keratopathy visual acuity grade obtained, a second recommended modification to the dose of the component of the B-cell disorder therapy; and cause output of at least one of: the recommended modification to the dose of the component of the B-cell disorder therapy; or the second recommended modification to the dose of the component of the B-cell disorder therapy.

Clause 102. The system of any one of clauses 89-101, wherein the first computing device is configured to cause output of the recommended modification to the dose of the component of the B-cell disorder therapy at least by displaying, at the user interface, the recommended modification to the dose of the component of the B-cell disorder therapy.

Clause 103. The system of any one of clauses 89-102, wherein the first computing device is further configured to send, to the second computing device ophthalmic examination data comprising at least one of: visual acuity assessment data associated with one or more visual acuity assessments of the patient; or corneal assessment data associated with one or more corneal assessments of the patient.

Clause 104. The system of any one of clauses 89-103, wherein the second computing device is configured to use at least one machine learning model that is trained at least on ophthalmic examination data and configured to determine at least one of: one or more recommended doses of the component of the B-cell disorder therapy; or one or more recommended modifications to the dose of the component of the B cell disorder therapy.

Clause 105. The system of any one of clause 104, wherein the at least one machine learning model is further configured to determine at least one of: a confidence score for at least one recommended dose of the one or more recommended doses; or a confidence score for at least one recommended modification of the one or more recommended modifications.

Clause 106. The system of any one of clauses 89-105, wherein the first computing device is configured to: display the plurality of predefined and selectable visual acuity assessment options on a first screen of the user interface; and display the plurality of predefined and selectable corneal assessment options on a second screen of the user interface that is different than the first screen.

Clause 107. The system of any one of clauses 89-106, wherein the second computing device is further configured to read machine-readable data included in instructions for administering a therapeutically effective dose of the component of the B-cell disorder therapy to the patient; and navigate, based on the machine-readable data read by the computing device, to a computer-executable application configured to: output, at a display of the second computing device, the keratopathy visual acuity grade received; and receive user input indicating at least one of: the recommended dose of the component of the B-cell disorder therapy; or recommended modification to the dose of the component of the B-cell disorder therapy.

Clause 108. The system of any one of clauses 89-107, wherein the second computing device is configured to provide, to the first computing device in real-time or near real-time during the ophthalmic examination or during a medical consultation with the patient, at least one of: the recommended dose of the component of the B-cell disorder therapy; or recommended modification to the dose of the component of the B-cell disorder therapy.

Clause 109. The system of any one of clauses 89-108, wherein the B-cell disorder therapy is a combination therapy.

Clause 110. The system of any one of clauses 89-109, wherein a therapeutically effective dose of the B-cell disorder therapy comprises a therapeutically effective dose of belantamab mafodotin.

Clause 111. The system of any one of clauses 89-109, wherein the B-cell disorder therapy is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab.

Clause 112. The system of any one of clauses 89-111, wherein the keratopathy visual acuity grade indicates a therapeutically effective dose of a component of a B-cell disorder therapy associated with the patient should be lower than a previously administered therapeutically effective dose of the component of the B-cell disorder therapy.

Clause 113. The system of any one of clauses 89-112, wherein at least one of the first computing device is configured to display, at the user interface, the keratopathy visual acuity grade determined.

Clause 114. The system of any one of clauses 89-113, wherein the first user input comprises first touch input received at a touchscreen of the computing device; and the second user input comprises second touch input received at the touchscreen.

In accordance with the above clauses, examples of methods, devices, systems, and non-transitory computer-readable media are provided.

A method for mitigating ocular toxicity comprising multiple steps may be performed. A first therapeutically effective dose of a component of a B-cell disorder therapy may be obtained. Instructions for administering the first therapeutically effective dose to a patient, wherein the instructions comprise an instruction to perform one or more ophthalmic examinations during the B-cell disorder therapy may be received. The first therapeutically effective dose may be administered in accordance with the instructions. An ophthalmic examination of a patient that has received the first therapeutically effective dose may be performed in accordance with the instructions and using a computing device. The ophthalmic examination may comprise a visual acuity assessment and a corneal assessment. A keratopathy visual acuity grade may be obtained by performing multiple steps. At least one visual acuity assessment option indicating a visual acuity of the patient may be selected based on the visual acuity assessment and from a plurality of predefined and selectable visual acuity assessment options presented at a user interface of the computing device. At least one corneal assessment option indicating a corneal state of the patient may be selected based on the corneal assessment and from a plurality of predefined and selectable corneal assessment options presented at the user interface. The keratopathy visual acuity grade may be caused to be determined automatically by the computing device based on the at least one visual acuity assessment option selected and based on the at least one corneal assessment option selected. A second therapeutically effective dose of the component of the B-cell disorder therapy may be administered to the patient based on the keratopathy visual acuity grade obtained. Machine-readable data included in the instructions may be read using the computing device. A computer-executable application may be navigated to based on the machine-readable data read by the computing device. The computer-executable application may be configured to perform multiple steps. The computer-executable application may be configured to display the plurality of predefined and selectable visual acuity assessment options and the plurality of predefined and selectable corneal assessment options at the user interface of the computing device. The computer-executable application may be configured to receive the user input indicating the selection of the at least one visual acuity assessment option and the user input indicating the selection of the at least one corneal assessment option The computer-executable application may be configured to automatically determine the keratopathy visual acuity grade. The machine-readable data may comprise an optical label that encodes an address of the computer-executable application. Reading the machine-readable data may comprise multiple steps including scanning the optical label using an optical scanner of the computing device and decoding the address. Navigating to the computer-executable application may comprise navigating to the address decoded from the optical label. The computer-executable application may be installed at the computing device. Determining the keratopathy visual acuity grade may comprise multiple steps including determining at least one visual acuity grade, determining at least one corneal grade, and determining the keratopathy visual acuity grade based on the visual acuity grade and based on the corneal grade. The B-cell disorder therapy may be a combination therapy. The first therapeutically effective dose of the component of the B-cell disorder therapy may comprise a therapeutically effective dose of belantamab mafodotin. The B-cell disorder therapy may be a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab. Selecting the at least one visual acuity assessment option may comprise multiple steps including selecting a first visual acuity assessment option for a first eye of the patient and selecting a second visual acuity assessment option for a second eye of the patient. The plurality of predefined and selectable corneal assessment options may comprise a plurality of predefined and selectable superficial keratopathy assessment options. Selecting the at least one corneal assessment option may comprise multiple steps including selecting a first superficial keratopathy assessment option for a first eye of the patient and selecting a second superficial keratopathy assessment option for a second eye of the patient. The plurality of predefined and selectable corneal assessment options may comprise a plurality of predefined and selectable corneal event options for each corneal event of a plurality of corneal events. Selecting the at least one corneal assessment option may comprise selecting a corneal event option for each eye of the patient and for each corneal event of the plurality of corneal events. The plurality of corneal events comprise at least one of microcyst-like deposits, sub-epithelial haze, stromal opacity, a corneal epithelial defect, a corneal ulcer or corneal erosion. A visual acuity unit may be selected from a plurality of predefined and selectable visual acuity unit options presented at the user interface. The plurality of predefined and selectable visual acuity assessment options presented at the user interface may be based on the visual acuity unit selected. The second therapeutically effective dose may be lower than the first therapeutically effective dose. A corneal assessment criteria option may be selected from a plurality of predefined and selectable corneal assessment criteria options presented at the user interface. The plurality of predefined and selectable corneal assessment options presented at the user interface may be based on the corneal assessment criteria option selected. A recommended modification to the first therapeutically effective dose may be caused to be determined automatically by the computing device based on the keratopathy visual acuity grade obtained. The recommended modification to the first therapeutically effective dose may be caused to be presented by the computing device at the user interface. At least one recommended modification to the first therapeutically effective dose may be caused to be determined automatically by the computing device based on output of a machine learning model trained at least on ophthalmic examination data and configured to provide at least one confidence score for at least one modification to a dose of the component of the B-cell disorder therapy. The at least one recommended modification to the first therapeutically effective dose and at least one corresponding confidence score for the at least one recommended modification to the first therapeutically effective dose may be caused to be presented by the computing device at the user interface. The plurality of predefined and selectable visual acuity assessment options and the plurality of predefined and selectable corneal assessment options may be presented at different screens of the user interface. A computing device may comprise one or more processors; and memory storing instructions that, when executed by the one or more processors, cause the computing device to perform one or more steps of the described method, additional operations and/or additional steps, and/or include additional elements. A system may comprise the computing device configured to perform one or more steps of the described method. The system may comprise one or more other computing devices respectively configured to perform one or more steps of the described method such that multiple computing devices collectively perform the steps of the described method. A computer-readable medium may store instructions, that when executed, cause performance of one or more steps of the described method, additional operations and/or additional steps, and/or include additional elements.

A computing device for mitigating ocular toxicity may be configured to perform multiple steps. The computing device may include one or more processors, a user interface, and memory storing instructions. The user interface may comprise a plurality of predefined and selectable visual acuity assessment options and a plurality of predefined and selectable corneal assessment options. The instructions, when executed by the one or more processors, may cause the computing device to perform multiple steps. The computing device may obtain a keratopathy visual acuity grade for a patient that has received a first therapeutically effective dose of a component of a B-cell disorder therapy by performing multiple steps. The computing device may receive first user input indicating a visual acuity of the patient based on a visual acuity assessment of the patient. The first user input may comprise a selection of at least one visual acuity assessment option of the plurality of predefined and selectable visual acuity assessment options. The computing device may receive second user input indicating a corneal state of the patient based on a corneal assessment of the patient. The second user input may comprise a selection of at least one corneal assessment option of the plurality of predefined and selectable corneal assessment options. The computing device may automatically determine the keratopathy visual acuity grade for the patient based on the at least one visual acuity assessment option selected and based on the at least one corneal assessment option selected. The computing device may present the keratopathy visual acuity grade obtained at the user interface. The computing device may read machine-readable data included in the instructions. The computing device may navigate to a computer-executable application based on the machine-readable data read. The computer-executable application may be configured to display the plurality of predefined and selectable visual acuity assessment options and the plurality of predefined and selectable corneal assessment options at the user interface. The computer-executable application may be configured to receive the user input indicating the selection of the at least one visual acuity assessment option and the user input indicating the selection of the at least one corneal assessment option. The computer-executable application may be configured to automatically determine the keratopathy visual acuity grade. The machine-readable data may comprise an optical label that encodes an address of the computer-executable application. The computing device may read the machine-readable data by performing multiple steps including scanning the optical label using an optical scanner of the computing device and decoding the address. The computing device may navigate to the computer-executable application at least by navigating to the address decoded from the optical label. The computing device may install the computer-executable application. The computing device may determine the keratopathy visual acuity grade by performing multiple steps including determining at least one visual acuity grade, determining at least one corneal grade, and determining the keratopathy visual acuity grade based on the visual acuity grade and based on the corneal grade. The B-cell disorder therapy may be a combination therapy. The first therapeutically effective dose of the component of the B-cell disorder therapy may comprise a therapeutically effective dose of belantamab mafodotin. The B-cell disorder therapy may be a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, rituximab or any combination therapy thereof. The computing device may receive selection of the at least one visual acuity assessment option by receiving user input indicating selection of a first visual acuity assessment option for a first eye of the patient and receiving user input indicating selection of a second visual acuity assessment option for a second eye of the patient. The plurality of predefined and selectable corneal assessment options may comprise a plurality of predefined and selectable superficial keratopathy assessment options. The computing device may receive selection of the at least one corneal assessment option by receiving user input indicating selection of a first superficial keratopathy assessment option for a first eye of the patient and receiving user input indicating selection of a second superficial keratopathy assessment option for a second eye of the patient. The plurality of predefined and selectable corneal assessment options may comprise a plurality of predefined and selectable corneal event options for each corneal event of a plurality of corneal events. The computing device may receive selection of the at least one corneal assessment option at least by receiving user input indicating selection of a corneal event option for each eye of the patient and for each corneal event of the plurality of corneal events. The plurality of corneal events may comprise at least one of microcyst-like deposits, sub-epithelial haze, stromal opacity, a corneal epithelial defect, or a corneal ulcer or corneal erosion. The user interface further comprises a plurality of predefined and selectable visual acuity units options. The computing device may receive user input indicating a visual acuity unit. The user input may comprise a selection of the visual acuity unit from the plurality of predefined and selectable visual acuity unit options. The computing device may present the plurality of predefined and selectable visual acuity assessment options at the user interface based on the visual acuity unit selected. The keratopathy visual acuity grade may indicate that a second therapeutically effective dose of the component of the B-cell disorder therapy should be lower than the first therapeutically effective dose. The user interface further may comprise a plurality of predefined and selectable corneal assessment criteria options. The computing device may receive user input indicating a corneal assessment criteria option. The user input may comprise a selection of the corneal assessment criteria option from the plurality of predefined and selectable corneal assessment criteria options. The computing device may present the plurality of predefined and selectable corneal assessment options at the user interface based on the corneal assessment criteria option selected. The computing device may automatically determine a recommended modification to the first therapeutically effective dose based on the keratopathy visual acuity grade obtained. The computing device may present the recommended modification to the first therapeutically effective dose at the user interface. The computing device may automatically determine at least one recommended modification to the first therapeutically effective dose based on output of a machine learning model trained at least on ophthalmic examination data and configured to provide at least one confidence score for at least one modification to a dose of the component of the B-cell disorder therapy. The computing device may present the at least one recommended modification to the first therapeutically effective dose and at least one corresponding confidence score for the at least one recommended modification to the first therapeutically effective dose at the user interface. The computing device may present the plurality of predefined and selectable visual acuity assessment options and the plurality of predefined and selectable corneal assessment options at different screens of the user interface.

Another method for mitigating ocular toxicity comprising multiple steps may be performed. A first therapeutically effective dose of a component of a B-cell disorder therapy may be provided. Instructions for administering the first therapeutically effective dose to a patient. The instructions may comprise an instruction to perform one or more ophthalmic examinations during the B-cell disorder therapy may be provided. The instructions may comprise machine-readable data that, when read by a computing device, cause the computing device to navigate to a computer-executable application. The computer-executable application may be configured to provide a keratopathy visual acuity grade based on a visual acuity assessment of an ophthalmic examination and based on a corneal assessment of the ophthalmic examination. The computer-executable application may be configured to provide the keratopathy visual acuity grade by performing multiple steps including receiving user input indicating a selection of at least one visual acuity assessment option indicating a visual acuity of the patient, receive user input indicating a selection of at least one corneal assessment option indicating a corneal state of the patient, and automatically determine the keratopathy visual acuity grade based on the at least one visual acuity assessment option selected and based on the at least one corneal assessment option selected. The at least one visual acuity assessment option may be selected from a plurality of predefined and selectable visual acuity assessment options presented at a user interface of the computing device. The computer-executable application may be configured to present the keratopathy visual acuity grade determined at the user interface of the computing device. The machine-readable data, when read by the computing device, may cause the computing device to navigate to the computer-executable application at least by navigating to a data store that stores the computer-executable application and is configured to send the computer-executable application to the computing device based on a request received at the data store from the computing device. The machine-readable data may comprise an optical label that encodes an address of the computer-executable application, and wherein the optical label. The optical label, when optically read by the computing device, may cause the computing device to decode the address of the computer-executable application. The computer-executable application may be configured to be installed at the computing device. The computer-executable application may be configured to determine the keratopathy visual acuity grade by performing multiple steps including determining at least one visual acuity grade, determining at least one corneal grade, and determining the keratopathy visual acuity grade based on the visual acuity grade and based on the corneal grade. The B-cell disorder therapy may be a combination therapy. The first therapeutically effective dose of the component of the B-cell disorder therapy may comprise a therapeutically effective dose of belantamab mafodotin. The B-cell disorder therapy may be a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab. The computer-executable application may be configured to receive the selection of the at least one visual acuity assessment option by performing multiple steps including receiving selection of a first visual acuity assessment option for a first eye of the patient and receiving selection of a second visual acuity assessment option for a second eye of the patient. The plurality of predefined and selectable corneal assessment options may comprise a plurality of predefined and selectable superficial keratopathy assessment options. The computer-executable application may be configured to receive the selection of the at least one visual acuity assessment option by performing multiple steps including receiving selection of a first superficial keratopathy assessment option for a first eye of the patient and receiving selection of a second superficial keratopathy assessment option for a second eye of the patient. The plurality of predefined and selectable corneal assessment options may comprise, for each corneal event of a plurality of corneal events, a plurality of predefined and selectable corneal event options. The computer-executable application may be configured to receive selection of the at least one corneal assessment option at least by receiving user input indicating a selection of a corneal event option or each eye of the patient and for each corneal event of the plurality of corneal events. The plurality of corneal events may comprise at least one of microcyst-like deposits, sub-epithelial haze, stromal opacity, a corneal epithelial defect, or a corneal ulcer or corneal erosion. The computer-executable application may be configured to receive user input indicating a selection of a visual acuity unit from a plurality of predefined and selectable visual acuity unit options presented at the user interface. The plurality of predefined and selectable visual acuity assessment options presented at the user interface may be based on the visual acuity unit selected. The keratopathy visual acuity grade may indicate a second therapeutically effective dose of the component of the B-cell disorder therapy should be lower than the first therapeutically effective dose. The computer-executable application may be configured to receive user input indicating a selection a corneal assessment criteria option from a plurality of predefined and selectable corneal assessment criteria options presented at the user interface. The plurality of predefined and selectable corneal assessment options presented at the user interface may be based on the corneal assessment criteria option selected. The computer-executable application may be configured to automatically determine a recommended modification to the first therapeutically effective dose based on the keratopathy visual acuity grade obtained. The computer-executable application may be configured to present the recommended modification to the first therapeutically effective dose at the user interface. The computer-executable application may be configured to automatically determine at least one recommended modification to the first therapeutically effective dose based on output of a machine learning model trained at least on ophthalmic examination data and configured to provide at least one confidence score for at least one modification to a dose of the component of the B-cell disorder therapy. The computer-executable application may be configured to present the at least one recommended modification to the first therapeutically effective dose and at least one corresponding confidence score for the at least one recommended modification to the first therapeutically effective dose at the user interface. The computer-executable application may be configured to present the plurality of predefined and selectable visual acuity assessment options and the plurality of predefined and selectable corneal assessment options are presented at different screens of the user interface. A computing device may comprise one or more processors; and memory storing instructions that, when executed by the one or more processors, cause the computing device to perform one or more steps of the described method, additional operations and/or additional steps, and/or include additional elements. A system may comprise the computing device configured to perform one or more steps of the described method. The system may comprise one or more other computing devices respectively configured to perform one or more steps of the described method such that multiple computing devices collectively perform the steps of the described method. A computer-readable medium may store instructions, that when executed, cause performance of one or more steps of the described method, additional operations and/or additional steps, and/or include additional elements.

A computing device may perform a method for mitigating ocular toxicity comprising multiple steps may be performed. The computing device may receive first B-cell disorder treatment data and first ophthalmic examination data. The first B-cell disorder treatment data comprises indication of one or more therapeutically effective doses of a component of a B-cell disorder therapy administered to one or more patients of a plurality of patients. The first ophthalmic examination data comprises, for one or more patients of the plurality of patients, one or more visual acuity assessments and one or more corneal assessments. The computing device may train a machine learning model configured to provide one or more recommendations for administration of the component of the B-cell disorder therapy based on the first B-cell disorder treatment data and based on the first ophthalmic examination data received. The computing device may receive second B-cell disorder treatment data associated with a patient being treated with the B-cell disorder therapy and second ophthalmic examination data associated with the patient. The second B-cell disorder treatment data comprises indication of one or more therapeutically effective doses of the component of the B-cell disorder therapy administered to the patient. The second ophthalmic examination data comprises one or more visual acuity assessments of the patient and one or more corneal assessments of the patient. The computing device may generate indication of at least one recommendation for administration of the component of the B-cell disorder therapy to the patient. The computing device may provide the indication of the at least one recommendation for administration of the component of the B-cell disorder therapy to a medical provider associated with administration of the B-cell disorder therapy. The component of the B-cell disorder therapy may comprise belantamab mafodotin. The first ophthalmic examination data may comprise one or more keratopathy visual acuity grades for each patient of the plurality of patients. Each keratopathy visual acuity grade of the one or more keratopathy visual acuity grades may be based on a visual acuity assessment and a corneal assessment. The computing device may generate, for each recommendation of the at least one recommendation, a confidence score of the recommendation using the machine learning model. The computing device may provide the confidence score for each corresponding recommendation of the at least one recommendation to the medical provider. The computing device may receive third ophthalmic examination data associated with an ophthalmic examination of the patient performed after a recommended dose has been administered to the patient in accordance with the at least one recommendation. The third ophthalmic data comprises a visual acuity assessment of the patient and a corneal assessment of the patient. The computing device may retrain the machine learning model based on the recommended dose administered to the patient and based on the third ophthalmic data. The computing device may receive patient data. The patient data may comprise indication of one or more characteristics of one or more patients of the plurality of patients. The computing device may train the machine learning model based on the patient data. The characteristics of the one or more patients may comprise at least one of a height of a patient, a weight of a patient, an age of a patient, a comorbidity of a patient, or a medical history of a patient. The one or more recommendations that the machine learning model is configured to provide may comprise at least one of a recommended dosage modification of the component of the B-cell disorder therapy or a recommend dose of the component of the B-cell disorder therapy. The B-cell disorder therapy may be a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab. A computing device may comprise one or more processors; and memory storing instructions that, when executed by the one or more processors, cause the computing device to perform one or more steps of the described method, additional operations and/or additional steps, and/or include additional elements. A system may comprise the computing device configured to perform one or more steps of the described method. The system may comprise one or more other computing devices respectively configured to perform one or more steps of the described method such that multiple computing devices collectively perform the steps of the described method. A computer-readable medium may store instructions, that when executed, cause performance of one or more steps of the described method, additional operations and/or additional steps, and/or include additional elements.

A computer-implemented method for mitigating ocular toxicity comprising multiple steps may be performed. A computing device may display, in association with an ophthalmic examination of a patient being treated for a B-cell disorder, a plurality of predefined and selectable visual acuity assessment options at a user interface of the computing device. The computing device may display, in association with the ophthalmic examination, a plurality of predefined and selectable corneal assessment options at the user interface. The computing device may receive, at the user interface and in association with a visual acuity assessment of the ophthalmic examination, first user input indicating selection, from the plurality of predefined and selectable visual acuity assessment options, of at least one visual acuity assessment option that indicates a visual acuity of the patient. The computing device may receive, at the user interface and in association with a corneal assessment of the ophthalmic examination, second user input indicating selection, from the plurality of predefined and selectable corneal assessment options, of at least one corneal assessment option that indicates a corneal state of the patient. The computing device may determine, automatically based on the at least one visual acuity assessment option selected and based on the at least one corneal assessment option selected, a keratopathy visual acuity grade. The computing device causes output of the keratopathy visual acuity grade determined. The computing device may read machine-readable data included in instructions for administering a therapeutically effective dose of a component of a B-cell disorder therapy to the patient. The computing device may navigate to a computer-executable application based on the machine-readable data read by the computing device. The computer-executable application may be configured to display the plurality of predefined and selectable visual acuity assessment options and the plurality of predefined and selectable corneal assessment options, receive the first user input and the second user input, and determine the keratopathy visual acuity grade. The machine-readable data may comprise an optical label that encodes an address of the computer-executable application. The computing device may read the machine-readable data at least by scanning the optical label using an optical scanner of the computing device and decoding the address. The computing device may navigate to the computer-executable application at least by navigating to the address decoded from the optical label. The computing device may install the computer-executable application. The computing device may determine the keratopathy visual acuity grade at least by determining at least one visual acuity grade, determining at least one corneal grade, and determining the keratopathy visual acuity grade based on the visual acuity grade and based on the corneal grade. The computing device may initiate determination of the at least one visual acuity grade based on a threshold quantity of visual acuity assessment options being selected. The computing device may initiate determination of the at least one corneal grade based on a threshold quantity of corneal assessment options being selected. The computing device may initiate determination of the keratopathy visual acuity grade based on the at least one visual acuity grade being determined and the at least one corneal grade being determined. The computing device may initiate determination of the keratopathy visual acuity grade based on a threshold quantity of visual acuity assessment options being selected and based on a threshold quantity of corneal assessment options being selected. The computing device may receive the first user input indicating selection of at least one visual acuity assessment option at least by receiving user input indicating selection of a first visual acuity assessment option for a first eye of the patient and receiving user input indicating selection of a second visual acuity assessment option for a second eye of the patient. The plurality of predefined and selectable corneal assessment options may include a plurality of predefined and selectable superficial keratopathy assessment options. The computing device may receive the second user input indicating selection of at least one corneal assessment option at least by receiving selection of a first superficial keratopathy assessment option for a first eye of the patient and receiving selection of a second superficial keratopathy assessment option for a second eye of the patient. The plurality of predefined and selectable corneal assessment options may include a plurality of predefined and selectable corneal event options for each corneal event of a plurality of corneal events. The computing device may receive the second user input indicating selection of at least one corneal assessment option at least by receiving user input indicating selection of a corneal event option for each eye of the patient and for each corneal event of the plurality of corneal events. The plurality of corneal events may include at least one of microcyst-like deposits, sub-epithelial haze, stromal opacity, a corneal epithelial defect, or a corneal ulcer or corneal erosion. The computing device may display, at the user interface and in association with the ophthalmic examination, a plurality of predefined and selectable visual acuity unit options. The computing device may receive, at the user interface, third user input indicating selection, from the plurality of predefined and selectable visual acuity unit options, of a visual acuity unit. The computing device may configure, based on the visual acuity unit selected, the plurality of predefined and selectable visual acuity assessment options displayed. The computing device may display, at the user interface and in association with the ophthalmic examination, a plurality of predefined and selectable corneal assessment criteria options. The computing device may receive, at the user interface, third user input indicating selection, from the plurality of predefined and selectable corneal assessment criteria options, of a corneal assessment criteria option. The computing device may configure based on the corneal assessment criteria option selected, the plurality of predefined and selectable corneal assessment options displayed. The computing device may determine automatically based on the keratopathy visual acuity grade obtained, a recommended modification to a therapeutically effective dose of a component of a B-cell disorder therapy associated with the patient. The computing device may cause output of the recommended modification to the first therapeutically effective dose. The computing device may cause output of the keratopathy visual acuity grade at least by sending the keratopathy visual acuity grade to a second computing device in signal communication with the computing device. The computing device may send ophthalmic examination data to a second computing device in signal communication with the computing device. The ophthalmic examination data may include at least one of visual acuity assessment data associated with one or more visual acuity assessments of the patient or corneal assessment data associated with one or more corneal assessments of the patient. The computing device may receive, from a second computing device in signal communication with the computing device, at least one of a recommended dose of a component of a B-cell disorder therapy associated with the patient or a recommended modification to the dose of the component of the B-cell disorder therapy. The second computing device is configured to use at least one machine learning model that is trained at least on ophthalmic examination data and configured to determine at least one of one or more recommended doses of the component of the B-cell disorder therapy or one or more recommended modifications to the dose of the component of the B-cell disorder therapy. The at least one machine learning model is further configured to determine at least one of a confidence score for at least one recommended dose of the one or more recommended doses or a confidence score for at least one recommended modification of the one or more recommended modifications. The computing device may display the plurality of predefined and selectable visual acuity assessment options on a first screen of the user interface. The computing device may display the plurality of predefined and selectable corneal assessment options on a second screen of the user interface that is different than the first screen. A B-cell disorder therapy associated with the patient is a combination therapy. A therapeutically effective dose of a B-cell disorder therapy associated with the patient comprises a therapeutically effective dose of belantamab mafodotin. A B-cell disorder therapy associated with the patient is a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab. The keratopathy visual acuity grade may indicate a therapeutically effective dose of a component of a B-cell disorder therapy associated with the patient should be lower than a previously administered therapeutically effective dose of the component of the B-cell disorder therapy. The computing device may cause output of the keratopathy visual acuity grade at least by displaying, at the user interface, the keratopathy visual acuity grade determined. The first user input comprises first touch input received at a touchscreen of the computing device, and the second user input comprises second touch input received at the touchscreen.

A system comprising a first computing device and a second computing device may be configured to respectively perform multiple steps. The first computing device may be configured to display, at a user interface of the computing device and in association with an ophthalmic examination of a patient being treated for a B-cell disorder, a plurality of predefined and selectable visual acuity assessment options. The first computing device may be configured to display, at the user interface and in association with the ophthalmic examination, a plurality of predefined and selectable corneal assessment options. The first computing device may be configured to receive, at the user interface and in association with a visual acuity assessment of the ophthalmic examination, first user input indicating selection, from the plurality of predefined and selectable visual acuity assessment options, of at least one visual acuity assessment option that indicates a visual acuity of the patient. The first computing device may be configured to receive, at the user interface and in association with a corneal assessment of the ophthalmic examination, second user input indicating selection, from the plurality of predefined and selectable corneal assessment options, of at least one corneal assessment option that indicates a corneal state of the patient. The first computing device may be configured to determine, automatically based on the at least one visual acuity assessment option selected and based on the at least one corneal assessment option selected, a keratopathy visual acuity grade. The first computing device may be configured to send, to the second computing device, the keratopathy visual acuity grade determined The second computing device may be configured to receive, from the first computing device, the keratopathy visual acuity grade determined. The second computing device may be configured to send, to the first computing device, at least one of a recommended dose of a component of a B-cell disorder therapy associated with the patient, or a recommended modification to the dose of the component of the B-cell disorder therapy. The first computing device may be configured to read machine-readable data included in instructions for administering a therapeutically effective dose of the component of the B-cell disorder therapy to the patient. The first computing device may be configured to navigate to a computer-executable application based on the machine-readable data read by the first computing device. The computer-executable application may be configured to display the plurality of predefined and selectable visual acuity assessment options and the plurality of predefined and selectable corneal assessment options, receive the first user input and the second user input, and determine the keratopathy visual acuity grade. The machine-readable data comprises an optical label that encodes an address of the computer-executable application. The first computing device may be configured to read the machine-readable data at least by scanning, using an optical scanner of the computing device, the optical label and decoding the address. The first computing device may be configured to navigate to the computer-executable application at least by navigating to the address decoded from the optical label. The first computing device may be further configured to install the computer-executable application. The first computing device is configured to automatically determine the keratopathy visual acuity grade at least by determining at least one visual acuity grade, determining at least one corneal grade, and determining the keratopathy visual acuity grade based on the visual acuity grade and based on the corneal grade. The first computing device may be configured to initiate determination of the at least one visual acuity grade based on a threshold quantity of visual acuity assessment options being selected. The first computing device may be configured to initiate determination of at least one corneal grade based on a threshold quantity of corneal assessment options being selected. The first computing device may be configured to initiate determination of the keratopathy visual acuity grade based on the at least one visual acuity grade being determined and the at least one corneal grade being determined. The first computing device may be configured to initiate determination of the keratopathy visual acuity grade based on a threshold quantity of visual acuity assessment options being selected and based on a threshold quantity of corneal assessment options being selected. The first computing device may be configured to receive the first user input indicating selection of at least one visual acuity assessment option at least by receiving user input indicating selection of a first visual acuity assessment option for a first eye of the patient and receiving user input indicating selection of a second visual acuity assessment option for a second eye of the patient. The plurality of predefined and selectable corneal assessment options comprise a plurality of predefined and selectable superficial keratopathy assessment options. The first computing device may be configured to receive the second user input indicating selection of at least one corneal assessment option at least by receiving, for a first eye of the patient, selection of a first superficial keratopathy assessment option, and receiving, for a second eye of the patient, selection of a second superficial keratopathy assessment option. The plurality of predefined and selectable corneal assessment options comprise, for each corneal event of a plurality of corneal events, a plurality of predefined and selectable corneal event options. The first computing device is configured to receive the second user input indicating selection of at least one corneal assessment option at least by receiving user input indicating selection of a corneal event option for each eye of the patient and for each corneal event of the plurality of corneal events. The plurality of corneal events comprise at least one of microcyst-like deposits, sub-epithelial haze, stromal opacity, a corneal epithelial defect, or a corneal ulcer or corneal erosion. The first computing device may be further configured to display, in association with the ophthalmic examination, a plurality of predefined and selectable visual acuity unit options at the user interface. The first computing device may be further configured to receive, at the user interface, third user input indicating selection of a visual acuity unit from the plurality of predefined and selectable visual acuity unit options. The first computing device may be further configured to configure the plurality of predefined and selectable visual acuity assessment options displayed based on the visual acuity unit selected. The first computing device may be further configured to display, in association with the ophthalmic examination, a plurality of predefined and selectable corneal assessment criteria options at the user interface. The first computing device may be further configured to receive, at the user interface, third user input indicating selection of a corneal assessment criteria option from the plurality of predefined and selectable corneal assessment criteria options. The first computing device may be further configured to configure the plurality of predefined and selectable corneal assessment options displayed based on the corneal assessment criteria option selected. The first computing device may be further configured to automatically determine a second recommended modification to the dose of the component of the B-cell disorder therapy based on the keratopathy visual acuity grade obtained. The first computing device may be further configured to cause output of at least one of the recommended modification to the dose of the component of the B-cell disorder therapy, or the second recommended modification to the dose of the component of the B-cell disorder therapy. The first computing device may be configured to cause output of the recommended modification to the dose of the component of the B-cell disorder therapy at least by displaying, at the user interface, the recommended modification to the dose of the component of the B-cell disorder therapy. The first computing device may be further configured to send, to the second computing device ophthalmic examination data. The ophthalmic examination data may include at least one of visual acuity assessment data associated with one or more visual acuity assessments of the patient or corneal assessment data associated with one or more corneal assessments of the patient. The second computing device may be configured to use at least one machine learning model that is trained at least on ophthalmic examination data and configured to determine at least one of one or more recommended doses of the component of the B-cell disorder therapy or one or more recommended modifications to the dose of the component of the B-cell disorder therapy. The at least one machine learning model may be further configured to determine at least one of a confidence score for at least one recommended dose of the one or more recommended doses or a confidence score for at least one recommended modification of the one or more recommended modifications. The first computing device may be further configured to display the plurality of predefined and selectable visual acuity assessment options on a first screen of the user interface and display the plurality of predefined and selectable corneal assessment options on a second screen of the user interface that is different than the first screen. The second computing device may be further configured to read machine-readable data included in instructions for administering a therapeutically effective dose of the component of the B-cell disorder therapy to the patient. The second computing device may be navigate to a computer-executable application based on the machine-readable data read by the computing device. The computer-executable application may be configured to output the keratopathy visual acuity grade received at a display of the second computing device. The computer-executable application may be configured to receive user input indicating at least one of the recommended dose of the component of the B-cell disorder therapy or the recommended modification to the dose of the component of the B-cell disorder therapy. The second computing device may be configured to provide, to the first computing device in real-time or near real-time during the ophthalmic examination or during a medical consultation with the patient, at least one of the recommended dose of the component of the B-cell disorder therapy or the recommended modification to the dose of the component of the B-cell disorder therapy. The B-cell disorder therapy may be a combination therapy. A therapeutically effective dose of the B-cell disorder therapy may include a therapeutically effective dose of belantamab mafodotin. The B-cell disorder therapy may be a cancer treatment therapy selected from the group consisting of: afatinib, bortezomib, ceritinib, crizotinib, dabrafenib, dasatinib, erlotinib, gefitinib, imatinib, nilotinib, trametinib, vandetanib, vemurafenib, cetuximab, ipilimumab, panitimumab, pertuzumab, and rituximab. The keratopathy visual acuity grade may indicate a therapeutically effective dose of a component of a B-cell disorder therapy associated with the patient should be lower than a previously administered therapeutically effective dose of the component of the B-cell disorder therapy. The first computing device may be configured to display the keratopathy visual acuity grade determined at the user interface. The first user input may include first touch input received at a touchscreen of the computing device, and the second user input may include second touch input received at the touchscreen.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosures. For example, any of the steps described above may be omitted, performed in alternative sequences, and/or performed in parallel (e.g., on different computing devices) in order to achieve similar results in a manner that is more appropriate to the requirements or preferences of a specific implementation. Further, although examples are described above, features and/or steps may be combined, divided, omitted, rearranged, revised, and/or augmented in any desired manner. Various alterations, modifications, and improvements will be appreciated with the benefit of this disclosure. Those alternations, modifications, and improvements are intended to be part of these disclosures, even if not expressly stated, and are intended to be within the spirt and scope of these disclosures. The disclosures herein should be considered in all respects as illustrative and not restrictive. As such, the scope of the claims should not be determined by these illustrative disclosures but rather by the appended claims and their equivalents.

1.-23. (canceled)

24. A computer-implemented method of mitigating ocular toxicity in a patient being treated for multiple myeloma, wherein the treatment for multiple myeloma comprises administration of belantamab mafodotin, the method comprising:
displaying, by a computing device at a user interface of the computing device and in association with an ophthalmic examination of the patient, a plurality of predefined and selectable visual acuity assessment options;
displaying, by the computing device at the user interface and in association with the ophthalmic examination, a plurality of predefined and selectable corneal assessment options;
receiving, by the computing device at the user interface and in association with a visual acuity assessment of the ophthalmic examination, first user input indicating selection, from the plurality of predefined and selectable visual acuity assessment options, of at least one visual acuity assessment option that indicates a visual acuity of the patient;
receiving, by the computing device at the user interface and in association with a corneal assessment of the ophthalmic examination, second user input indicating selection, from the plurality of predefined and selectable corneal assessment options, of at least one corneal assessment option that indicates a corneal state of the patient;
determining, automatically by the computing device based on the at least one visual acuity assessment option selected and based on the at least one corneal assessment option selected, a keratopathy visual acuity grade; and
causing, by the computing device, output of the keratopathy visual acuity grade determined.

25. The computer-implemented method of claim 24, further comprising:
reading, by the computing device, machine-readable data included in instructions for administering a therapeutically effective dose of belantamab mafodotin to the patient; and
navigating, by the computing device based on the machine-readable data read by the computing device, to a computer-executable application configured to:
display the plurality of predefined and selectable visual acuity assessment options and the plurality of predefined and selectable corneal assessment options;
receive the first user input and the second user input; and
determine the keratopathy visual acuity grade.

26. (canceled)

27. The computer-implemented method of claim 24, wherein the automatically determining the keratopathy visual acuity grade comprises:
determining at least one visual acuity grade;
determining at least one corneal grade; and
determining the keratopathy visual acuity grade based on the visual acuity grade and based on the corneal grade;
optionally further comprising:
initiating the determining the at least one visual acuity grade based on a threshold quantity of visual acuity assessment options being selected;
initiating the determining the at least one corneal grade based on a threshold quantity of corneal assessment options being selected; and
initiating the determining the keratopathy visual acuity grade based on the at least one visual acuity grade being determined and the at least one corneal grade being determined.

28. The computer-implemented method of claim 24, further comprising initiating the determining the keratopathy visual acuity grade based on a threshold quantity of visual acuity assessment options being selected and based on a threshold quantity of corneal assessment options being selected.

29. The computer-implemented method of claim 24, wherein:
(a) the receiving the first user input indicating selection of at least one visual acuity assessment option comprises:
receiving, for a first eye of the patient, user input indicating selection of a first visual acuity assessment option; and
receiving, for a second eye of the patient, user input indicating selection of a second visual acuity assessment option;
(b) the plurality of predefined and selectable corneal assessment options comprise a plurality of predefined and selectable superficial keratopathy assessment options; and
the receiving the second user input indicating selection of at least one corneal assessment option comprises:
receiving, for a first eye of the patient, selection of a first superficial keratopathy assessment option; and
receiving, for a second eye of the patient, selection of a second superficial keratopathy assessment option;
(c) the plurality of predefined and selectable corneal assessment options comprise, for each corneal event of a plurality of corneal events, a plurality of predefined and selectable corneal event options;
receiving the second user input indicating selection of at least one corneal assessment option comprises receiving, for each eye of the patient and for each corneal event of the plurality of corneal events, user input indicating selection of a corneal event option; and
the plurality of corneal events comprise at least one of:
microcyst-like deposits;
sub-epithelial haze;
stromal opacity;
a corneal epithelial defect; or
a corneal ulcer or corneal erosion;
(d) further comprising:
displaying, by the computing device at the user interface and in association with the ophthalmic examination, a plurality of predefined and selectable visual acuity unit options;
receiving, by the computing device at the user interface, third user input indicating selection, from the plurality of predefined and selectable visual acuity unit options, of a visual acuity unit; and
configuring, by the computing device and based on the visual acuity unit selected, the plurality of predefined and selectable visual acuity assessment options displayed;
(e) further comprising:
displaying, by the computing device at the user interface and in association with the ophthalmic examination, a plurality of predefined and selectable corneal assessment criteria options;
receiving, by the computing device at the user interface, third user input indicating selection, from the plurality of predefined and selectable corneal assessment criteria options, of a corneal assessment criteria option; and
configuring, by the computing device and based on the corneal assessment criteria option selected, the plurality of predefined and selectable corneal assessment options displayed;
(f) further comprising:
determining, automatically by the computing device based on the keratopathy visual acuity grade obtained, a recommended modification to a therapeutically effective dose of a component of a B-cell disorder therapy associated with the patient; and
causing, by the computing device, output of the recommended modification to the first therapeutically effective dose;
(g) causing output of the keratopathy visual acuity grade comprises sending, to a second computing device in signal communication with the computing device, the keratopathy visual acuity grade; or
(h) further comprising sending, by the computing device to a second computing device in signal communication with the computing device, ophthalmic examination data comprising at least one of:
visual acuity assessment data associated with one or more visual acuity assessments of the patient; or
corneal assessment data associated with one or more corneal assessments of the patient.

30. The computer-implemented method of claim 24, further comprising receiving, from a second computing device in signal communication with the computing device, at least one of:
a recommended dose of belantamab mafodotin; or
a recommended modification to the dose of belantamab mafodotin;
optionally wherein the second computing device is configured to use at least one machine learning model that is trained at least on ophthalmic examination data and configured to determine at least one of:
one or more recommended doses of belantamab mafodotin; or
one or more recommended modifications to the dose of belantamab mafodotin;
optionally wherein the at least one machine learning model is further configured to determine at least one of:
a confidence score for at least one recommended dose of the one or more recommended doses; or
a confidence score for at least one recommended modification of the one or more recommended modifications.

31. The computer-implemented method of claim 24 wherein:
the displaying the plurality of predefined and selectable visual acuity assessment options comprises displaying the plurality of predefined and selectable visual acuity assessment options on a first screen of the user interface; and
the displaying the plurality of predefined and selectable corneal assessment options comprises displaying the plurality of predefined and selectable corneal assessment options on a second screen of the user interface that is different than the first screen.

32. (canceled)

33. The computer-implemented method of claim 24, wherein:
(a) the keratopathy visual acuity grade indicates a therapeutically effective dose of belantamab mafodotin should be lower than a previously administered therapeutically effective dose of belantamab mafodotin;
(b) causing output of the keratopathy visual acuity grade comprises displaying, at the user interface of the computing device, the keratopathy visual acuity grade determined; or
(c) the first user input comprises first touch input received at a touchscreen of the computing device; and the second user input comprises second touch input received at the touchscreen.

34. A computing device comprising:
one or more processors; and
memory storing instructions that, when executed by the one or more processors, cause the computing device to perform the method of claim 24.

35. A system comprising:
a first computing device configured to perform the method of claim 24; and
a second computing device configured to receive, from the first computing device, at least one of ophthalmic examination data associated with the ophthalmic examination or the keratopathy visual acuity grade.

36. A non-transitory computer-readable medium storing instructions that, when executed, cause performance of the method of claim 24.

37. A system comprising:
a first computing device; and
a second computing device;
wherein the first computing device is configured to:
display, at a user interface of the computing device and in association with an ophthalmic examination of a patient being treated for multiple myeloma, wherein the treatment for multiple myeloma comprises administration of belantamab mafodotin, a plurality of predefined and selectable visual acuity assessment options;
display, at the user interface and in association with the ophthalmic examination, a plurality of predefined and selectable corneal assessment options;
receive, at the user interface and in association with a visual acuity assessment of the ophthalmic examination, first user input indicating selection, from the plurality of predefined and selectable visual acuity assessment options, of at least one visual acuity assessment option that indicates a visual acuity of the patient;
receive, at the user interface and in association with a corneal assessment of the ophthalmic examination, second user input indicating selection, from the plurality of predefined and selectable corneal assessment options, of at least one corneal assessment option that indicates a corneal state of the patient;
determine, automatically based on the at least one visual acuity assessment option selected and based on the at least one corneal assessment option selected, a keratopathy visual acuity grade; and
send, to the second computing device, the keratopathy visual acuity grade determined;
wherein the second computing device is configured to:
receive, from the first computing device, the keratopathy visual acuity grade determined; and
send, to the first computing device, at least one of:
a recommended dose of belantamab mafodotin; or
a recommended modification to the dose of belantamab mafodotin.

38. The system of claim 37, wherein the first computing device is further configured to:
read machine-readable data included in instructions for administering a therapeutically effective dose of belantamab mafodotin to the patient; and
navigate, based on the machine-readable data read by the first computing device, to a computer-executable application configured to:
display the plurality of predefined and selectable visual acuity assessment options and the plurality of predefined and selectable corneal assessment options;
receive the first user input and the second user input; and
determine the keratopathy visual acuity grade.

39. (canceled)

40. The system of claim 37, wherein:
(a) the first computing device is configured to automatically determine the keratopathy visual acuity grade at least by:
determining at least one visual acuity grade;
determining at least one corneal grade; and
determining the keratopathy visual acuity grade based on the visual acuity grade and based on the corneal grade;
(b) the first computing device is further configured to:
initiate determination of the at least one visual acuity grade based on a threshold quantity of visual acuity assessment options being selected;
initiate determination of at least one corneal grade based on a threshold quantity of corneal assessment options being selected; and
initiate determination of the keratopathy visual acuity grade based on the at least one visual acuity grade being determined and the at least one corneal grade being determined;
(c) the first computing device is further configured to initiate determination of the keratopathy visual acuity grade based on a threshold quantity of visual acuity assessment options being selected and based on a threshold quantity of corneal assessment options being selected;
(d) the first computing device is configured to receive the first user input indicating selection of at least one visual acuity assessment option at least by:
receiving, for a first eye of the patient, user input indicating selection of a first visual acuity assessment option; and
receiving, for a second eye of the patient, user input indicating selection of a second visual acuity assessment option;
(e) the plurality of predefined and selectable corneal assessment options comprise a plurality of predefined and selectable superficial keratopathy assessment options; and
the first computing device is configured to receive the second user input indicating selection of at least one corneal assessment option at least by:
receiving, for a first eye of the patient, selection of a first superficial keratopathy assessment option; and
receiving, for a second eye of the patient, selection of a second superficial keratopathy assessment option;
(f) the plurality of predefined and selectable corneal assessment options comprise, for each corneal event of a plurality of corneal events, a plurality of predefined and selectable corneal event options;
the first computing device is configured to receive the second user input indicating selection of at least one corneal assessment option at least by receiving, for each eye of the patient and for each corneal event of the plurality of corneal events, user input indicating selection of a corneal event option; and
the plurality of corneal events comprise at least one of:
microcyst-like deposits;
sub-epithelial haze;
stromal opacity;
a corneal epithelial defect; or
a corneal ulcer or corneal erosion;
(g) the first computing device is further configured to:
display, at the user interface and in association with the ophthalmic examination, a plurality of predefined and selectable visual acuity unit options;
receive, at the user interface, third user input indicating selection, from the plurality of predefined and selectable visual acuity unit options, of a visual acuity unit; and
configure, based on the visual acuity unit selected, the plurality of predefined and selectable visual acuity assessment options displayed;
(h) the first computing device is further configured to:
display, at the user interface and in association with the ophthalmic examination, a plurality of predefined and selectable corneal assessment criteria options;
receive, at the user interface, third user input indicating selection, from the plurality of predefined and selectable corneal assessment criteria options, of a corneal assessment criteria option; and
configure, based on the corneal assessment criteria option selected, the plurality of predefined and selectable corneal assessment options displayed;
(i) the first computing device is further configured to:
determine, automatically based on the keratopathy visual acuity grade obtained, a second recommended modification to the dose of belantamab mafodotin; and
cause output of at least one of:
the recommended modification to the dose of belantamab mafodotin; or
the second recommended modification to the dose of belantamab mafodotin;
(j) the first computing device is configured to cause output of the recommended modification to the dose of belantamab mafodotin at least by displaying, at the user interface, the recommended modification to the dose of the component of the B-cell disorder therapy; or
(k) the first computing device is further configured to send, to the second computing device ophthalmic examination data comprising at least one of:
visual acuity assessment data associated with one or more visual acuity assessments of the patient; or
corneal assessment data associated with one or more corneal assessments of the patient.

41. The system of claim 37, wherein the second computing device is configured to use at least one machine learning model that is trained at least on ophthalmic examination data and configured to determine at least one of:
one or more recommended doses of belantamab mafodotin; or
one or more recommended modifications to the dose of belantamab mafodotin;
optionally wherein the at least one machine learning model is further configured to determine at least one of:
a confidence score for at least one recommended dose of the one or more recommended doses; or
a confidence score for at least one recommended modification of the one or more recommended modifications.

42. The system of claim 37, wherein:
(a) the first computing device is further configured to:
display the plurality of predefined and selectable visual acuity assessment options on a first screen of the user interface; and
display the plurality of predefined and selectable corneal assessment options on a second screen of the user interface that is different than the first screen;
(b) the second computing device is further configured to:
read machine-readable data included in instructions for administering a therapeutically effective dose of the component of the B-cell disorder therapy to the patient; and
navigate, based on the machine-readable data read by the computing device, to a computer-executable application configured to:
output, at a display of the second computing device, the keratopathy visual acuity grade received; and
receive user input indicating at least one of:
the recommended dose of belantamab mafodotin; or
the recommended modification to the dose of belantamab mafodotin;
(c) the second computing device is configured to provide, to the first computing device in real-time or near real-time during the ophthalmic examination or during a medical consultation with the patient, at least one of:
the recommended dose of belantamab mafodotin; or
the recommended modification to the dose of belantamab mafodotin;
(d) the keratopathy visual acuity grade indicates a therapeutically effective dose of belantamab mafodotin associated with the patient should be lower than a previously administered therapeutically effective dose of belantamab mafodotin;
(h) the first computing device is configured to display, at the user interface, the keratopathy visual acuity grade determined; or
(i) the first user input comprises first touch input received at a touchscreen of the computing device; and the second user input comprises second touch input received at the touchscreen.
